SEQUENCE LISTING (128 sequences)

SEQ ID NO: 1 - protein, 310 aa, Artificial Sequence
  Synthetic Peptide
MNINILLSTYNGERFLAEQIQSIQRQTVNDWTLLIRDDGSTDGTQDIIRTFVKEDKRIQWINEGQTENLGVIKNFYTLLKHQKADVYFFSDQDDIWLDNKLEVTLLEAQKHEMTAPLLVYTDLKVVTQHLAVCHDSMIKTQSGHANTSLLQELTENTVTGGTMMITHALAEEWTTCDGLLMHDWYLALLASAIGKLVYLDIPTELYRQHDANVLGARTWSKRMKNWLTPHHLVNKYWWLITSSQKQAQLLLDLPLKPNDHELVTAYVSLLDMPFTKRLATLKRYGFRKNRIFHTFIFRSLVVTLFGYRRK

SEQ ID NO: 2 - protein, 581 aa, Artificial Sequence
  Synthetic Peptide
MNRILLYVHFNKYNKISAHVYYQLEQMRSLFSKIVFISNSKVSHEDLKRLKNHCLIDEFLQRKNKGFDFSAWHDGLIIMGFDKLEEFDSLTIMNDTCFGPIWEMAPYFENFEEKETVDFWGITNNRGTKAFKEHVQSYFMTFKNQVIQNKVFQQFWQSIIEYENVQEVIQHYETQLTSILLNEGFSYQTVFDTRKAESSFMPHPDFSYYNPTAILKHHVPFIKVKAIDANQHIAPYLLNLIRETTNYPIDLIVSHMSQISLPDTKYLLSQKYLNCQRLAKQTCQKVAVHLHVFYVDLLDEFLTAFENWNFHYDLFITTDSDIKRKEIKEILQRKGKTADIRVTGNRGRDIYPMLLLKDKLSQYDYIGHFHTKKSKEADFWAGESWRKELIDMLVKPADSILSAFETDDIGIIIADIPSFFRFNKIVNAWNEHLIAQEMMSLWRKMDVKKQIDFQAMDTFVMSYGTFVWFKYDALKSLFDLELTQNDIPSEPLPQNSILHAIERLLVYIAWGDSYDFRIVKNPYELTPFIDNKLLNLREDEGAHTYVNFNQMGGIKGALKYIIVGPAKAMKYIFLRLMEKLK

SEQ ID NO: 3 - protein, 301 aa, Artificial Sequence
  Synthetic Peptide
MHSSDQKRVAVLMATYNGECWIEEQLKSIIEQKDVDISIFISDDLSTDNTLNICEEFQLSYPSIINILPSVNKFGGAGKNFYRLIKDVDLENYDYICFSDQDDIWYKDKIKNAIDCLVFNNANCYSSNVIAYYPSGRKNLVDKAQSQTQFDYFFEAAGPGCTYVIKKETLIEFKKFIINNKNAAQDICLHDWFLYSFARTRNYSWYIDRKPTMLYRQHENNQVGANISFKAKYKRLGLVRNKWYRKEVTKIANALADDSFVNNQLGKGYIGNLILALSFWKLRRKKADKIYILLMLILNIF

SEQ ID NO: 4 - protein, 313 aa, Artificial Sequence
  Synthetic Peptide
MKVNILMATYNGEKFLAQQIESIQKQTFKEWNLLIRDDGSSDKTCDIIRNFTAKDSRIRFINENEHHNLGVIKSFFTLVNYEVADFYFFSDQDDVWLPEKLSVSLEAAKHKASDVPLLVYTDLKVVNQELNILQDSMIRAQSHHANTTLLPELTENTVTGGTMMINHALAEKWFTPNDILMHDWFLALLAASLGEIIYLDLPTQLYRQHDNNVLGARTMDKRFKILREGPKSIFTRYWKLIHDSQKQASLIVDKYGDIMTANDLELIKCFIKIDKQPFMTRLRWLWKYGYSKNQFKHQVVFKWLIATNYYNKR

SEQ ID NO: 5 - protein, 310 aa, Artificial Sequence
  Synthetic Peptide
MNINILLSTYNGERFLAEQIQSIQKQTIKDWTLLIRDDGSTDRTPDIIREFVKQDQRIQWINENQIENLGVIKNFYTLLKYQAADVYFFSDQDDIWLEDKLEVTLLEAQKHDLSKPLLVYTDLKVVNQQLEITHASMIKTQSAHANTTLLQELTENTVTGGTMMINQALAKEWNTCEGLLMHDWYLALVAAARGKLVCLDIPTELYRQHDANVLGARTWSKRMKHWLRPHQLIRKYWWLITSSQQQAQLLLDLPLQPKDRDMVEAYVSLLTMSLTKRLATLKTYGFRKNRAFHTLVFWSLVITLFGYRRK

SEQ ID NO: 6 - protein, 310 aa, Artificial Sequence
  Synthetic Peptide
MNINILLSTYNGERFLAEQIQSIQKQTIKDWTLLIRDDGSTDRTPDIIREFVKQDQRIQWINENQIENLGVIKNFYTLLKYQAADVYFFSDQDDIWLEDKLEVTLLEAQKHDLSKPLLVYTDLKVVNQQLEITHASMIKTQSAHANTTLLQELTENTVTGGTMMINQALAKEWNTCEGLLMHDWYLALVAAARGKLVYLDIPTELYRQHDANVLGARTWSKRMKHWLRPHQLIRKYWWLITSSQQQAQLLLDLPLQPKDRDMVEAYVSLLTMSLTKRLATLKTYGFRKNRAFHTLVFWSLVITLFGYRRK

SEQ ID NO: 7 - protein, 311 aa, Artificial Sequence
  Synthetic Peptide
MKVNILMSTYNGQEFIAQQIQSIQKQTFENWNLLIRDDGSSDGTPKIIADFAKSDARIRFINADKRENFGVIKNFYTLLKYEKADYYFFSDQDDVWLPQKLELTLASVEKENNQIPLMVYTDLTVVDRDLQVLHDSMIKTQSHHANTSLLEELTENTVTGGTMMVNHCLAKQWKQCYDDLIMHDWYLALLAASLGKLIYLDETTELYRQHESNVLGARTWSKRLKNWLRPHRLVKKYWWLVTSSQQQASHLLELDLPAANKAIIRAYVTLLDQSFLNRIKWLKQYGFAKNRAFHTFVFKTLIITKFGYRRK

SEQ ID NO: 8 - protein, 317 aa, Artificial Sequence
  Synthetic Peptide
MKINILMSTYNGEKFLAEQIESIQKQTVTDWTLLIRDDGSSDRTPEIIQDFVAKDSRIHFINADHRINFGVIKNFFTLLKYEEADYYFFSDQDDVWLPHKIETSLNKAKELEKNRPFLIYTDLTIVNQSLETIHESMISFQSDHANTTLLEELTENTVTGGTALINHALAELWTDDKDLLMHDWFLALLASAMGNLVYINEATELYRQHDRNVLGARTWSKRLKTWSKPHLMLNKYWWLIQSSQQQAQKLLDLPLSSDKRKLVEHYVTLLEKPLMTRLRDLKKYGYKKNRAFHTFVFRMLIITKIGYRRTVKNGIIQ

SEQ ID NO: 9 - protein, 581 aa, Artificial Sequence
  Synthetic Peptide
MNRVLLYVHFNKYNKVSKHIYYQLEKLRPLFTTVVFISNSKVEQKELENLQKQRLIDSFIQRENKGFDFAAWHDGMMKIGFDDLTLCDSLTIMNDTCFGPLWGMAPYFEKFDNNQSVDFWGLTNNRKTSSFKEHIQSYFITFKQHVIQSDAFLNFWKTIKEYDDVQEVIQKYETQVTTTLLEAGFNYQTVFDTREADSSFMLHPDFSYYNPTAILQHRVPFIKVKAIDANQHITPYLLNMIEEETTYPVDLIISHMSQVGLPDAKYLLARKYLPFESLVTQNVPRIAVHLHVFYVDLLNEFLEGFASWEFQYDLYITTDTQEKKEAIEKLLVQSNRHAHLYVTGNVGRDVLPMLLLKDKLRDYDYIGHFHTKKSKEADFWAGESWRKELINMLIKPANEIVRSFENNDIGIVIADIPSFFRFNKIVDAWNEHLIAPEMMRLWKEMGLKKEIDFQSMDTFVMSYGTFVWFKFDALKPLFDLDLTVDDIPKEPLPQNSILHAIERLLVYIAWDRFYDFRIVKNPYNLSPFIDNKLLNLRESGGARTYVNFDHMGGIKGALKYIIIGPARAMKYIVKRVLKSKR

SEQ ID NO: 10 - protein, 330 aa, Artificial Sequence
  Synthetic Peptide
MNRVLLYVHFNKYNKVSKHIYYQLEKLRPLFTTVVFISNSKVEQKELENLQKQRLIDSFIQRENKGFDFAAWHDGMMKIGFDDLTLCDSLTIMNDTCFGPLWGMAPYFEKFDNNQSVDFWGLTNNRKTSSFKEHIQSYFITFKQHVIQSDAFLNFWKTIKEYDDVQEVIQKYETQVTTTLLEAGFNYQTVFDTREADSSFMLHPDFSYYNPTAILQHRVPFIKVKAIDANQHITPYLLNMIEEETTYPVDLIISHMSQVGLPDAKYLLARKYLPFESLVTQNVPRIAVHLHVFYVDLLNEFLEGFASWEFQYDLYITTDTQEKRKQLKNY

SEQ ID NO: 11 - protein, 274 aa, Artificial Sequence
  Synthetic Peptide
MGVSVRPLYYNRYSRKKEAIEKLLVQSNRHAHLYVTGNVGRDVLPMLLLKDKLRDYDYIGHFHTKKSKEADFWAGESWRKELINMLIKPANEIVRSFENNDIGIVIADIPSFFRFNKIVDAWNEHLIAPEMMRLWKEMGLKKEIDFQSMDTFVMSYGTFVWFKFDALKPLFDLDLTVDDIPKEPLPQNSILHAIERLLVYIAWDRFYDFRIVKNPYNLSPFIDNKLLNLRESGGARTYVNFDHMGGIKGALKYIIIGPARAMKYIVKRVLKSKR

SEQ ID NO: 12 - protein, 408 aa, Artificial Sequence
  Synthetic Peptide
MIGKIIRSYQDEGGRATLRKIRQRLQGGGHPQSAGKIDLNRIPIMPQLEDIAQADYINHPYQRPAKLDKKQLNIAWVSPPVGKGGGGHTTISRFVKYLQSQGHHITFYIYHNNTIEQSAKEAQEIFSKAYGIEVAVDDLKNFSNQDLVFATSWETAYAVFNLKSENLHKFYFVQDFEPIFYGVGSRYKLAEATYKFGFYGITAGKWLTHKLKDYHMDADYFNFGADTDIYKPKAPLQKKKKIAFYARAHTERRGFELGVMALKIFKDKHPEYDIEFFGQDMSHYDIPFDFIDRGILNKEELAAIYHESVACLVLSLTNVSLLPLELLVAGCIPVMNSGDNNTMVLGENDDIAYAEAYPVALAEELCKAVERSDIDTYANEMSQKYDGVSWENSYRKVEEIIRREVIND

SEQ ID NO: 13 - protein, 327 aa, Artificial Sequence
  Synthetic Peptide
MTDKIKATVFIPVYNGENDHLEETLTALYTQKTDFSWNVMITDSESKDRSVAIIETFAERYGNLQLIKLKKSDYSHGATRQMAAELSSAEYMVYLSQDAVPANEHWLAEMLKPFTIHHDIVAVLGKQKPRIGCFPAMKYDINAVFNEQGVAGAITLWTRQEESLKGKYTKESFYSDVCSAAPRDFLVNEIGYRSVPYSEDYEYGKDILDAGYMKAYNSDAIVEHSNDVLLSEYKQRIFDETYNVRRNSGVTTPISVSTVLIQFLKSSVKDAMKIVSDQDYSWKRKLYWLAVNPLFHFEKWRGMRLANSVDMTKDNSKHSLENSKSKG

SEQ ID NO: 14 - protein, 585 aa, Artificial Sequence
  Synthetic Peptide
MKRLLLYVHFNKYNRLSPHVLYQLKKMRPLFSNLIFISNSSLNDSDRQELLSSGLVNEVIQRQNIGFDFAAWRDGMATVGFESLSEYDNVTIMNDTCFGPLWDMKPYFLTYEDDEEVDFWGLTNNRQTKEFDEHIQSYFISFKKTVLSNETFLHFWRTVQDFTDVQDVIKNYETQVTTGLLKEGFRYKCIFNTVTADASGMLHADFSYYNPTAILKHQVPFIKVKTIDANQSIAPYLLQVIKNQTDYPVDLIVSHMSDIHYPDAPYLLSQKYLEKQEESDLKVSEHSIAVHLHVFYVDLLEEFLHAFTSFKFPFDLYITTDKSEKESEIKAILDSFRVSAKIVVTGNIGRDVLPMLKLKDELSQYDYIGHFHTKKSKEADFWAGESWRNELIDMLIKPANTIINQFEDPAIGIIIADIPSFFRFNKIVTPLNEHLIAPEMNKLWEKMNLSKTIDFEQFDTFVMSYGTFVWFKYDALKPLFDLNLKDGDVPKEPLPQNSILHAVERLLIYIAWDSHFDFRIAKNNVELTPFLDNKLLNDKSNSLPNTYVDFTYMGGIKGALKYIFIGPARAIKYIYIRTKEKIFNG

SEQ ID NO: 15 - protein, 583 aa, Artificial Sequence
  Synthetic Peptide
MKRLLLYVHFNKYNRVSSHVVYQLTQMRSLFSKVIFISNSQVADADVKMLREKHLIDDFIQRQNSGFDFAAWRDGMVFVGFDELVTYDSVTTMNDTCFGPLWEMYSIYQEFETKTTVDFWGLTNNRATKSFREHIQSYFISFKASVLRSTAFRDFWENIKEYQDVQKVIDQYETKVTTTLLDAGFQYDVVFDTTKEDASHMLHADFSYYNPTAILNHRVPFIKVKAIDNNQHITPYLLNDIQKNSTYPIDLIVSHMSEINYPDFSYLLGHKYVKKRERVDLKNQKVAVHLHVFYVDLLEEFLTAFKQFHFSYDLFITTDSDDKKAEIEEILSANGQEAQVFVTGNIGRDVLPMLKLKNYLSAYDFVGHFHTKKSKEADFWAGQSWREELIDMLVKPADNILAQLQQNPKIGLVIADMPTFFRYNKIVDAWNEHLIAPEMNTLWQKMGMTKKIDFNAFHTFVMSYGTFVWFKYDALKPLFDLNLTDDDVPEEPLPQNSILHAIERLLIYIAWNEHYDFRISKNPVDLTPFIDNKLLNERGNSAPNTFVDFNYMGGIKGAFKYIFIGPARAVKYILKRSLQKIKS

SEQ ID NO: 16 - protein, 304 aa, Artificial Sequence
  Synthetic Peptide
MLENTKILRKVFYLWQKGELMILITGSNGQLGTELRYLLDERGVDYVAVDVAEMDITNEDKVEAVFAQVKPTLVYHCAAYTAVDAAEDEGKALNEAINVTGSENIAKACGKYGATLVYISTDYVFDGNKPVGQEWVETDHPDPKTEYGRTKRLGELAVERYAEHFYIIRTAWVFGNYGKNFVFTMEQLAENHSRLTVVNDQHGRPTWTRTLAEFMCYLTENQKAFGYYHLSNDAKEDTTWYDFAKEILKDKAVEVVPVDSSAFPAKAKRPLNSTMNLDKAKATGFVIPTWQEALKAFYQQGLKK

SEQ ID NO: 17 - protein, 824 aa, Artificial Sequence
  Synthetic Peptide
MIKDTFLKTNWLNISHHIILLVFGFYFSFYSLAKELVSSTAQPVNYYAHLLNVSFVGYIISLIGLSYYLSRQVSRQLFLKTSFIVISYLIVSYWVQITQHLNDKRFDIWSLTKNQFYQFQALPSLLIILVMATLIKILVAYFAIEKDRFGLLGYQGNTFSVALILAVVPINDIHLLKLISSRFSELVTAGNSQIALLKISGLLIVLLVIFATIIYVVLNALKHLKSNKPSFSVAATTSLFLALVFNYTFQYGVKGDEALLGYYVFPGATLFQIVAITLVALLAYVITNRYWPTTFFLLILGTIISVVNDLKESMRSEPLLVTDFVWLQELGLVTSFVKKSVIVEMVVGLAICIVVAWYLHGRVLAGKLFMSPVKRASAVLGLFIVSCSMLIPFSYEKEGKILSGLPIISALNNDNDINWLGFSTNARYKSLAYVWTRQVTKKIMEKPTNYSQETIASIAQKYQKLAEDINKDRKNNIADQTVIYLLSESLSDPDRVSNVTVSHDVLPNIKAIKNSTTAGLMQSDSYGGGTANMEFQTLTSLPFYNFSSSVSVLYSEVFPKMAKPHTISEFYQGKNRIAMHPASANNFNRKTVYSNLGFSKFLALSGSKDKFKNIENVGLLTSDKTVYNNILSLINPSESQFFSVITMQNHIPWSSDYPEEIVAEGKNFTEEENHNLTSYARLLSFTDKETRAFLEKLTQINKPITVVFYGDHLPGLYPDSAFNKHIENKYLTDYFIWSNGTNEKKNHPLINSSDFTAALFEHTDSKVSPYYALLTEVLNKASVDKSPDSPEVKAIQNDLKNIQYDVTIGKGYLLKHKTFFKISR

SEQ ID NO: 18 - protein, 284 aa, Artificial Sequence
  Synthetic Peptide
MILITGANGQLGSELRHLLDERTQEYVAVDVAEMDITNAEMVDKVFEEVKPSLVYHCAAYTAVDAAEDEGKELDFAINVTGTENVAKAAAKHDATLVYISTDYVFDGEKPVGQEWEVDDLPDPKTEYGRTKRMGEELVEKYASKFYTIRTAWVFGNYGKNFVFTMQNLAKTHKTLTVVNDQHGRPTWTRTLAEFMTYLAENQKDFGYYHLSNDAKEDTTWYDFAVEILKDTDVEVKPVDSSQFPAKAKRPLNSTMSLEKAKATGFVIPTWQDALKEFYKQEVKK

SEQ ID NO: 19 - protein, 284 aa, Artificial Sequence
  Synthetic Peptide
MILITGSNGQLGTELRYLLDERHVDYVAVDVAEMDITDADKVEAVFAQVKPTLVYHCAAYTAVDAAEDEGKALNEAINVTGSENIAKACGKYGATLVYISTDYVFDGNKPVGQEWLETDVPDPQTEYGRTKRLGELAVEQYAEHFYIIRTAWVFGNYGKNFVFTMQQLAEKHPRLTVVNDQHGRPTWTRTLAEFMCYLAENQKAFGYYHLSNDAKEDTTWYDFAKEILKDKAVEVVPVDSSAFPAKAKRPLNSTMNLDKAKATGFVIPTWQEALKEFYQQDRHQ

SEQ ID NO: 20 - protein, 284 aa, Artificial Sequence
  Synthetic Peptide
MILITGSNGQLGTELRYLLDERHVDYVAVDVAEMDITDADKVEAVFAQVKPTLVYHCAAYTAVDAAEDEGKALNEAINVTGSENIAKACGKYGATLVYISTDYVFDGNKPVGQEWLETDVPDPQTEYGRTKRLGELAVEQYAEHFYIIRTAWVFGNYGKNFVFTMQQLAEKHPRLTVVNDQHGRPTWTRTLAEFMCYLAENQKAFGYYHLSNDAKEDTTWYDFAKEILKDKAIEVVPVDSSAFPAKAKRPLNSTMNLDKAKATGFVIPTWQEALKEFYQQDRHQ

SEQ ID NO: 21 - protein, 284 aa, Artificial Sequence
  Synthetic Peptide
MILITGSNGQLGTELRHLLNERNEDYVAVDVAEMDITKAEKVDEVFLQVKPSLVYHCAAYTAVDAAEDEGKELDYAINVTGTENIAKACEKYNATLVYISTDYVFDGEKPVGQEWEVDDKPDPKTEYGRTKRLGEEAVEKYVKNFYIIRTAWVFGNYGKNFVFTMQHLAKSHNSLTVVNDQHGRPTWTRTLAEFMTYLAENQKEYGYYHLSNDATEDTTWYDFALEILKDTDVVVKPVDSSQFPAKAKRPLNSTMSLTKAKATGFVIPTWQEALQEFYKQDVKK

SEQ ID NO: 22 - protein, 284 aa, Artificial Sequence
  Synthetic Peptide
MILITGSNGQLGTELRYLLDERNVEYVAVDVAEMDITNPDMVDEVFAQVKPTLVYHCAAYTAVDAAEDEGKALNQAINVDGTVNIAKACQKYNATLVYISTDYVFDGTKTVGQEWLETDIPDPKTEYGRTKRLGEEAVEKYVDQFYIIRTAWVFGHYGKNFVFTMQNLAKTHPKLTVVNDQYGRPTWTRTLAEFMCHLTENQKDYGYYHLSNDSKEDTSWYDFAKEILKDTDVEVVPVDSSAFPAKAKRPLNSTMNLDKAKATGFVIPTWQEALNEFYKQEVKK

SEQ ID NO: 23 - protein, 267 aa, Artificial Sequence
  Synthetic Peptide
MNFLTKKNRILLREMVKTDFKLRYQGSAIGYLWSILKPLMMFTIMYLVFIRFLRLGGNIPHFPVALLLANVIWSFFSEATSMGMVSIVSRGDLLRKLNFSKHIIVFSAILGALINFLINLVVVLIFALINGVTISNYAYFSFFLFIELVVFVVGIALLLSTVFVYYRDLAQVWEVLLQAGMYATPIIYPITFVLEGHPLAAKILMLNPIAQMIQDFRYLLIDRANVTIWQMSTNWFYIAIPYLIPFILLFIGITVFKKNATKFAEII

SEQ ID NO: 24 - protein, 401 aa, Artificial Sequence
  Synthetic Peptide
MTNNKIAVKVEHVSKSFKLPTEATKSFRTTLVNRFRGIKGFTEQQVLKDINFEVHKGDFFGIVGRNGSGKSTLLKIISQIYVPEKGQVTVDGKMVSFIELGVGFNPELTGRENVYMNGAMLGFTKEEINAMYDDIVDFAELHDFMNQKLKNYSSGMQVRLAFSVAIKAQGDVLILDEVLAVGDEAFQRKCNDYFMERKDSGKTTILVTHDMGAVKKYCNRAVLIEDGLVKAYGEPFDVANQYSVDNTETKEELQDSEKVAISDIVQQLRVNLTSKQRITPKEIISFEVSYEVLRDEPTYIAFSLTDMDRNIWVYNDNSRDQLVEGIGKKTISYQCHLSHLNDIKLKLEVTVRDKDGQMLLFSTAEQSPKIIIQRDDITSDDFSALDSASGLYQRNGQWTFS

SEQ ID NO: 25 - protein, 335 aa, Artificial Sequence
  Synthetic Peptide
MHKVSIICTNYNKAPWLGEALDSFLNQKTNFEVDIIVIDDASTDESKTILEDYQTRFPEKITLLFNDHNLGITKTWIKACLYAKGKYIARCDGDDYWTDDLKLQKQVDALEASKYSKWSNTDFDFVDNKGKVLHSNVFETGYIPFTDTYEKVLALKGMTMASTWVVDAELMRFVNQKINIETPDDTFDMQLELFQLTSLTYINDSTTVYRMTSNSDSRPADKKRMIHRIKQLLQTQVFYLAKYPQANIPQIANLLMEQDGKNELRIHELSCLINDLRQELNEKTEQQKEREFEIKEIIENQSRQICELTHQYNCVINSRRWKYMSKLIDFIRRKK

SEQ ID NO: 26 - protein, 268 aa, Artificial Sequence
  Synthetic Peptide
MNFLTKKNRILLREMVKTDFKLRYQGSFIGHLWSILKPMLLFTIMYLVFVRFLKFDDGTPHYAVSLLLGMVTWNFFTEATNMGMLSIVSRGDLLRKINFPKEIIVISSVVGATINYFINILVVFAFALINGVQPSFGVFILIPLFLELFLFATGVAFILATLFVKYRDMGPIWEVMLQAGMYGTPIIYSITYIIQRGHLGIAKVMMMNPLAQIIQELRHFIVYSGATINWDIFENKFFTLIPIILSLSAFVIGYVIFKRNAKKFAEIL

SEQ ID NO: 27 - protein, 388 aa, Artificial Sequence
  Synthetic Peptide
MSEKKVVLSVDSVSKSFKLPTEASNSLRTSLVNYFKGIKGYTEQHVLDDISFQVEEGDFFGIVGRNGSGKSTLLKIISKIYEPEKGTVTVDGKLVPFIELGVGFNPELTGRENVFMNGALLGFSRDEVAAMYDDIVSFAELHDFMDQKLKNYSSGMQVRLAFSIAIKAKGDILILDEVLAVGDEAFQRKCFDYFAQLKREHKTVILVTHSMEQVQRFCNKAMLIDKGHHMEVGTPLEISQIYKQLNGLNVAKESAKETENNGISLSSQFINHKDDTLTFTFDVHFEQTIEDPVLTFTIHKDTGELLYRWVSDEEVEGSIMIKNHKVSIDFAIQNIFPNGKFTTEFGVKSRDRSKEYAMFSGICNFELINRGKSGNNIYWKPETTVKLS

SEQ ID NO: 28 - protein, 427 aa, Artificial Sequence
  Synthetic Peptide
MRMYQGKRFLLTHIWLRGFSGAEINILELATYLKEAGAQVEVFTFLAKSPMLDEFQKNGIPVIDDSDYPFDVSQYDVVCSAQNIIPPAMIEALGKSQEKLPKFIFFHMAALPEHVLEQPYIYQLEKKISSATLAISEEIVNKNLKRFFKDIPNLHYYPNPAPESYAAMEHLKKQSPERILVISNHPPQEVIDMEPLLAKKGIHVDYFGVWSDHYELVTPELLASYDCVVGIGKNAQYCLVMGKPIYIYDHFKGPGYLTETNFEAAALNNFSGRGFEEQEKTAEELVDDLLEHYQSAQAFQHNHLYDYRSRYTISTIVDHIYKSINIIPKAIAPLEQVDVEYIKAITLFIRTRLVRLENDVANLWEAVHRYEQLDRKATAKREALEQLLTAKTTELNLIKTSRMFKLYQLLWRIKGFFFRKEHLKRAK

SEQ ID NO: 29 - protein, 269 aa, Artificial Sequence
  Synthetic Peptide
MDFFSRKNRILLKELIKTDFKLRYQGSAIGYLWSILKPLMLFAIMYIVFVRFLPLGGDVPHWPVALLLGNVIWTFFQETTMMGMVSVVTRGDLLRKLNFSKQTIVFSAVSGAAINFGINVIVVLIFALLNGVTFTFRWNLFLLIPLFLELLLFSTGIAFILSTLYVRYRDIGPVWEVILQGGFYGTPIIYSLTYIATRSVVGAKLLLLSPIAQIIQDMRHILIDPANVTIWQMINHKSIAVIPYLVPIFVFIIGFLVFNYNAKKFAEII

SEQ ID NO: 30 - protein, 405 aa, Artificial Sequence
  Synthetic Peptide
MTKNNIAVKVDHVSKYFKLPVESTQSLRTALVNRFKGIKGYKKQHVLRDIDFEVEKGDFFGIVGRNGSGKSTLLKIISQIYVPEQGKVTVDGKLVSFIELGVGFNPELTGRENVYMNGAMLGFTTEEVDTMYQDIVDFAELQDFMNQKLKNYSSGMQVRLAFSVAIKAQGDVLILDEVLAVGDEAFQRKCNDYFLERKNSGKTTILVTHDMAAVKKYCNKAVLIDDGLIKAIGEPFDVANQYSLDNTDQIVEDKQEEEAAVQEEEQIVVDNLEVKLLSANRMTPRDSIRFEISYNVLADVGTYIALSLTDVDRNIWIYNDNSLDYLSSGSGKKRVFYECHLKSLNDIKLKLEVTVRDKQGQMLAFSSATNTPIISINRDDLEGDDKSAMDSASGLIQRNGQWQFS

SEQ ID NO: 31 - protein, 465 aa, Artificial Sequence
  Synthetic Peptide
MVKVSIICTNYNKGSWIGEAIDSFLKQETSFPYEIIIVDDASTDHSVHIIKTYQKQYPDLIRAFFNQENQGITKTWSDICKKARGQYIARCDGDDYWIDPFKLQKQIDLLETSPESKWSNTDFDMVDSKGNIIHKDVLKNNIIPFMDSYEKMLALKGMTMASTWLVETKLMLEINDRINKDAVDDTFNIQLELFKKTKLAFLRDSTTVYRMDAESDSRSKDSEKLAQRFDRLLETQLEYIEKYPDSDYKKVLEYLLPKHNDFEKVLAQDGKNVWDNQQITIYLAKGDDQEFSEENCFQFPLQHSGNIQLTFPENIRKIRIDLSEIPSYYRQVSLVNTTVNTELLPTWTNAKVFGYSYYFIAPDPQMIYDLTAQEGQDFKLTYEWFNVDQPSQPDFLANHLVKELDQKKVELKMLSPYKYQYQKAVAERDLYLEQLNEMVVRYNSVTHSRRWTIPTKIINLFRRKK

SEQ ID NO: 32 - protein, 267 aa, Artificial Sequence
  Synthetic Peptide
MELFSKKNRILLKELVKTDFKLRYQGSAIGYLWSILKPLLMFTIMYLVFIRFLRLGGSVPHFPVALLLANVIWSFFSEATGMGMVSIVTRGDLLRKLNFSKHTIVFSAVLGALINFSINLVVVLIFALINGVTISPFAYMAIPLFIELLILAVGVALLLSTLFVYYRDLAQVWEVLMQAAMYATPIIYPITFVSDKNPLAAKILMLNPLAQMIQDLRFLLIDRANATIWQMSNHWYYVMIPYLIPFLVLALGILVFNKNAKKFAEII

SEQ ID NO: 33 - protein, 403 aa, Artificial Sequence
  Synthetic Peptide
MSTRDIAVKVEHVSKSFKLPTEATKSFRTTLVNRFRGIKGYTEQKVLKDINFEVKKGDFFGIVGRNGSGKSTLLKIISQIYVPEKGTVTVEGKMVSFIELGVGFNPELTGRENVYMNGAMLGFTQEEVDAMYEDIVDFAELHDFMNQKLKNYSSGMQVRLAFSVAIKAQGDVLILDEVLAVGDEAFQRKCNDYFMERKESGKTTILVTHDMAAVKKYCNRAVLIEDGLVKALGDPDDVANQYSFDNAIASETVEKKEDGKSTEKKESQLISDFSAQLLTKPQISPDEDITISFSYNVLKNMETHVALSFIDIDTNLGLYNDNSMSLKTNGQGQKTVTMTCQMSYLNHAKLKLAATVRDKDKHPLAFLPVNEIPVILIDRKVDASNESEWDANTGILRRSSQWT

SEQ ID NO: 34 - protein, 590 aa, Artificial Sequence
  Synthetic Peptide
MKKILFVSPTGTLDNGAEISITNLMVLLTQEGYDIINVIPKIKHSTHDAYLHKMRENQIKVYELDYTNWWWESAPGDKIGHLEDRSAYYQKYIYEIRKIIAEEAVDLVITSTANLFQGALAAACERIPHYWIIHEFPLDEFAYYKELIPFIEEYSDKIFTVEGKLTEFLRPLLKESQKLFPFVPFVNIKKNNNLKTGEETRLISISRINENKNQLELLKAYQSMAEPKPELLFVGDWDDSYKEKCDDFIQSHQLKTVRFLGHQSNPWNLMTDKDILVLNSKMETFGLVFVEALIQGIPVLASNNYGYSSVVDYFGCGKLYHLGDEKELVALLNEFVTNFSEEKKKSLTQSFMVEEKYTIEKSYCALLDAISNENSVKSDRPIWLSQFLGAYNPLSTFSPAGKESISIYYRDENGNWSENQKLVFSLFNRDSFTFSVPKGMTRIRLDMSERPSYYDKITLVDSDTMTQLLPTNVSGFEENNSFYFNHSDPQMEFNVSFSKNNVFQLSYQLANLENIFQDSFLPNQLVQKLLSFKEKQSDLEMLKIENHQLQEKNKLKQEQLEEMVVRYNSVIHSRRWSIPTKMINFLRRKK

SEQ ID NO: 35 - protein, 846 aa, Artificial Sequence
  Synthetic Peptide
MKQLKKIWDMLGKQKLLIFIFIFALNVTLRNYDLLIGRRANSSLSFKVISKNFDIMIEHWEALPSHFKIIGGVCLVIYVLSILGLSFYLSKNLKKTFFIELLLGYGLYIVISYFLAVTRELNNESFKIWDLAKNHFFQPYFLPTLVLIIVCTLALNYLIRVKMKRSHLSRKMTLLLENFSETEFLLTGLIVSFILSDTLYVKLLQESLRAYYHKPLAYESLLFLYTLLTLILFSVIVEACFNAYRSIKLNRPNLSLAFVSSLLFATIFNYAFQYGLKNDADLLGKYIVPGATAYQILVLTAAGFFLYLIINRYLLVTFLIVILGSIITVVNVLKVGMRNEPLLVTDFAWVTNIRLLARSVNANIIFSTLLILAALILLYLFLRKRLLQGKITENHRLKVGLISSICLLGFSIFIIFRNEKGSKIVNGIPVISQVNNWVDIGYQGFYSNASYKSLMYVWTKQVTKSIMDKPSDYSKERILKLAKKYNNVANKINKVRTENISNQTVIYILSESFSDPDRVKGVNLSRDVIPNIKQIKEKTTSGLMHSDGYGGGTANMEFQSLTGLPYYNFNSSVSTLYTEVVPDMSVFPSISNQFKSKNRVVIHPSSASNYSRKYVYDKLKFPTFVASSGTSDKITHSEKVGLNVSDKTTYQNILDKINPSQSQFFSVMTMQNHVPWASDEPSDVVATGKGYTKDENGSLSSYARLLTYTDKETKDFLAQLSQLKHKVTVVFYGDHLPGLYPESAFKKDPDSQYQTDYFIWSNYNTKTLNHSYVNSSDFTAELLEHTNSKVSPYYALLTEVLDNTTVGHGKLTKEQKEIANDLKLIQYDITVGKGYIRNYKGFFDIR

SEQ ID NO: 36 - protein, 390 aa, Artificial Sequence
  Synthetic Peptide
MKQSVYIIGSKGIPAKYGGFETFVEKLTEYQKDGNIQYYVACMRENSAKSGFTADTFEYNGAICYNIDVPNIGPARAIAYDIAAVNKAIELSKGNKDEAPIFYILACRIGPFISGLKKKIRSIGGRLLVNPDGHEWLRAKWSLPVRKYWKFSEQLMVKHADLLVCDSKNIEKYIREDYKQYQPKTTYIAYGTDTTPSSLKSEDAKVRNWYREKGVSENGYYLVVGRFVPENNYETMIREFIKSKSNKDFVLITNVEQNKFYDQLLKETGFDKDLRVKFVGTVYDQELLKYIRENAFAYFHGHEVGGTNPSLLEALASTKLNLLLDVGFNREVGEDGAIYWKKDELAHVIEEVERFDEGDITELDEKSSQRIADAFTWEKIVSDYEEVFTV

SEQ ID NO: 37 - protein, 282 aa, Artificial Sequence
  Synthetic Peptide
MNKYCILVLFNPDISVFIDNVKKILSLDVSLFVYDNSANKHAFLALSSQEQTKINYFSICENIGLSKAYNETLRHILEFNKNVKNKSINDSVLFLDQDSEVDLNSINILFETISAAESNVMIVAGNPIRRDGLPYIDYPHTVNNVKFVISSYAVYRLDAFRNIGLFQEDFFIDHIDSDFCSRLIKSNYQILLRKDAFFYQPIGIKPFNLCGRYLFPIPSQHRTYFQIRNAFLSYRRNGVTFNFLFREIVNRLIMSIFSGLNEKDLLKRLHLYLKGIKDGLKM

SEQ ID NO: 38 - protein, 264 aa, Artificial Sequence
  Synthetic Peptide
MVYIIIVSHGHEDYIKKLLENLNADDEHYKIIVRDNKDSLLLKQICQHYAGLDYISGGVYGFGHNNNIAVAYVKEKYRPADDDYILFLNPDIIMKHDDLLTYIKYVESKRYAFSTLCLFRDEAKSLHDYSVRKFPVLSDFIVSFMLGINKTKIPKESIYSDTVVDWCAGSFMLVRFSDFVRVNGFDQGYFMYCEDIDLCLRLSLAGVRLHYVPAFHAIHYAHHDNRSFFSKAFRWHLKSTFRYLARKRILSNRNFDRISSVFHP

SEQ ID NO: 39 - protein, 301 aa, Artificial Sequence
  Synthetic Peptide
MVAVTYSPGPHLERFLASLSLATERPVSVLLADNGSTDGTPQAAVQRYPNVRLLPTGANLGYGTAVNRTIAQLGEMAGDAGEPWGDDWVIVANPDVQWGPGSIDALLDAASRWPRAGALGPLIRDPDGSVYPSARQMPSLIRGGMHAVLGPFWPRNPWTTAYRQERLEPSERPVGWLSGSCLLVRRSAFGQVGGFDERYFMYMEDVDLGDRLGKAGWLSVYVPSAEVLHHKAHSTGRDPASHLAAHHKSTYIFLADRHSGWWRAPLRWTLRGSLALRSHLMVRSSLRRSRRRKLKLVEGRH

SEQ ID NO: 40 - protein, 296 aa, Artificial Sequence
  Synthetic Peptide
MNSNIYAVIVTYNPELKNLNALITELKEQNCYVVVVDNRTNFTLKDKLADIEKVHLICLGRNEGIAKAQNIGIRYSLEKGAEKIIFFDQDSRIRNEFIKKLSCYMDNENAKIAGPVFIDRDKSHYYPICNIKKNGLREKIHVTEGQTPFKSSVTISSGTMVSKEVFEIVGMMDEELFIDYVDTEWCLRCLNYGILVHIIPDIEMVHAIGDKSVKICGINIPIHSPVRRYYRVRNAFLLLRKNHVPLLLSIREVVFSLIHTTLIIATQKNKIEYMKKHILATLDGIRGITGGGRYNA

SEQ ID NO: 41 - protein, 289 aa, Artificial Sequence
  Synthetic Peptide
MDISIIIVNYNTPKLTVEAIESILKSKTKYSYEIIVVDNHSSDDSVRILKGKFPNIVVIENKQNVGFSKANNQAIKLSKGRYILLLNSDTIVKEDTIEKMIEFMDKSKKVGASGCEVVLPNGELDRACHRGFPTPEASFYYLVGLARLFPRSRRFNQYHLGYMNLNEPHPIDCLVGAFMMVRREVIEQVGLLDEEFFMYGEDIDWCYRIKQAGWEIYYCPFTSIIHYKGASSKKKPFKIVYEFHRAMFLFHRKHYARKYPFIVNCLVYTGIAAKFILSAIINTFRKIGG

SEQ ID NO: 42 - protein, 377 aa, Artificial Sequence
  Synthetic Peptide
MKISIIGNTANAMILFRLDLIKTLTKKGISVYAFATDYNDSSKEIIKKAGAIPVDYNLSRSGINLAGDLWNTYLLSKKLKKIKPDAILSFFSKPSIFGSLAGIFSGVKNNTAMLEGLGFLFTEQPHGTPLKTKLLKNIQVLLYKIIFPHINSLILLNKDDYHDLIDKYKIKLKSCHILGGIGLDMNNYCKSTPPTNEISFIFIARLLAEKGVNEFVLAAKKIKKTHPNVEFIILGAIDKENPGGLSESDVDTLIKSGVISYPGFVSNVADWIEKSSVFVLPSYYREGVPRSTQEAMAMGRPILTTNLPGCKETIIDGVNGYVVKKWSHEDLAEKMLKLINNPEKIISMGEESYKLARERFDANVNNVKLLKILGIPD

SEQ ID NO: 43 - protein, 471 aa, Artificial Sequence
  Synthetic Peptide
MVKVIRGRERFLTKLYAFVDFAMMQGAFFLAWVLKFKVFHNGVGGHLPLEDYLFWSFVYGAIAIVIGYLVELYAPKRKEKFSNELAKVLQVHTLSMFVLLSVLFTFKTVDVSRSFLLLYFAWNLILVSIYRYIVKQSLRTLRKKGYNKQFVLIIGAGSIGRKYFENLQMHPEFGLEVVGFLDDFRTKHAPEFAHYKPIIGQTADLEHVLSHQLIDEVIVALPLQAYPKYREIIAVCEKMGVRVSIIPDFYDILPAAPHFEIFGDLPIINVRDVPLDELRNRVLKRSFDIVFSLVAIIVTSPIMLLIAIGIKLTSPGPIIFKQERVGLNRRTFYMYKFRSMKPMPQSVSDTQWTVESDPRRTKFGAFLRKTSLDELPQFFNVLKGDMSIVGPRPERPFFVEKFKKEIPKYMIKHHVRPGITGWAQVCGLRGDTSIQERIEHDLFYIENWSLWLDIKIILLTITNGLVNKNAY

SEQ ID NO: 44 - protein, 324 aa, Artificial Sequence
  Synthetic Peptide
MEMPLVSIVVATYFPRTDFFEKQLQSLNNQTYENIEIIICDDSANDAEYEKVKKMVENIISRFPCKVIRNEKNVGSNKTFERLTQEANGDYICYCDQDDIWLSEKVERLVNHITKHHCTLVYSDLSLIDENDRIIHKSFKRSNFRLKHVHGDNTFAHLINRNSVTGCAMMIRADVAKSAIPFPDYDEFVHDHWLAIHAAVKGSLGYIKEPLVWYRIHLGNQIGNQRLVNITNINDYIRHRIEKQGNKYRLTLERLSLTLQQKQLVYFQIHLTEARKKFSQKPCLGNFFKIVPLIKYDIILFLFELMIFTVPFTCSIWIFKKLKY

SEQ ID NO: 45 - protein, 1127 aa, Artificial Sequence
  Synthetic Peptide
MERCRMNKKIPFDQYQRYKNAAEIINLIREENQSFTILEVGANEHRNLEHFLPKDQVTYLDIEVPEHLKHMTNYIEADATNMPLDDNAFDFVIALDVFEHIPPDKRNQFLFEINRVAKEGFLIAAPFNTEGVEETEIRVNEYYKALYGEGFRWLEEHRQYTLPNLEETEDILRKENIEYVKFEHGSLLFWEKLMRLHFLVADRNVLHDYRFMIDDFYNKNIYEVDYIGPCYRNFIVVCRDKAKREFIQSIYEKRKQNSYLKNSTISKLNELENSIYSLKIIDKENQIYKKSLEITEQLLEDLKLKEQQIIEKIQTIKKKTEMIELQNQKIQELKIECENKSIENNNLYSQLLEKENYIKQLQNQAESMRIKNRLKKILNFSFIKYVRKIINIIFRRKFKFKLQPVHHLEWSNGKWLVLGRDPHFILKGGSYPSSWTIIQWRASANSSALLRLYYDTGGGFSENQSFNLGKIGNDINRDYECVICLPENIHLLRLDIEGEISEFELENLTFTSISRLEVFYKSFINHCRKRNIKNYKELYSLIKKLFILVRREGLKSIWYRAKQKLSMELLSEDPYEVFLNVSSKVDKEIVLSEIKKLKYKPKFSVILPVYNVEEKWLRKCIDSVLNQWYPYWELCIVDDNSSKDYIKPVLEEYSNRDSRIKTVFRSNNGHISEASNTALEIATGDFIALLDHDDELAPEALYENAVLLNEHPDADMIYSDEDKITKDGKRHSPLFKPDWSPDTLRSQMYIGHLTVYRTNLVRQLGGFRKGFEGSQDYDLALRVAEKTNNIYHIPKILYSWREIETSTAVNPSSKPYAHEAGLKALNEHLERVFGKGKAWAEETEYLFVYDVRYAIPEDYPLVSIIIPTKDNIELLSSCIQSILDKTTYPNYEILIMNNNSVMEETYSWFDKQKENSKIRIIDAMYEFNWSKLNNHGIREANGEVFVFLNNDTIVISEDWLQRLVEKALREDVGTVGGLLLYEDNTIQHAGVVIGMGGWADHVYKGMHPVHNTSPFISPVINRNVSASTGACLAIAKKVIEKIGGFNEEFIICGSDVEISLRALKMGYVNIYDPYVRLYHLESKTRDSFIPERDFELSAKYYSPYREIGDPYYNQNLSYNHLIPTIRS

SEQ ID NO: 46 - protein, 310 aa, Artificial Sequence
  Synthetic Peptide
MARSGGVVIKKKVAAIIITYNPDLTILRESYTSLYKQVDKIILIDNNSTNYQELKKLFEKKEKIKIVPLSDNIGLAAAQNLGLNLAIKNNYTYAILFDQDSVLQDNGINSFFFEFEKLVSEEKLNIVAIGPSFFDEKTGRRFRPTKFIGPFLYPFRKITTKNPLTEVDFLIASGCFIKLECIKSAGMMTESLFIDYIDVEWSYRMRSYGYKLYIHNDIHMSHLVGESRVNLGLKTISLHGPLRRYYLFRNYISILKVRYIPLGYKIREGFFNIGRFLVSMIITKNRKTLILYTIKAIKDGINNEMGKYKG

SEQ ID NO: 47 - DNA, 39 bp, Artificial Sequence
  Synthetic Sequence
tacctcgagggcaaagccgtttttccataggctccgccc

SEQ ID NO: 48 - DNA, 39 bp, Artificial Sequence
  Synthetic Sequence
tacggatccgttatttcctcccgttaaataatagataac

SEQ ID NO: 49 - DNA, 36 bp, Artificial Sequence
  Synthetic Sequence
agactcgagatgcaggatgtttttatcattggtagc

SEQ ID NO: 50 - DNA, 37 bp, Artificial Sequence
  Synthetic Sequence
agactcgagatgttcatttaaaaataaagcctcgtac

SEQ ID NO: 51 - DNA, 36 bp, Artificial Sequence
  Synthetic Sequence
tctgaattcatgcaggatgtttttatcattggtagc

SEQ ID NO: 52 - DNA, 40 bp, Artificial Sequence
  Synthetic Sequence
acactgcagttaatgttcatttaaaaataaagcctcgtac

SEQ ID NO: 53 - DNA, 32 bp, Artificial Sequence
  Synthetic Sequence
cactctaacccagctggattgataaaaaagcg

SEQ ID NO: 54 - DNA, 31 bp, Artificial Sequence
  Synthetic Sequence
caatccagctgggttagagtggaaacggtct

SEQ ID NO: 55 - DNA, 35 bp, Artificial Sequence
  Synthetic Sequence
cgtaattatttgcaggaacaaagcgtcctaaaatg

SEQ ID NO: 56 - DNA, 32 bp, Artificial Sequence
  Synthetic Sequence
cgctttgttcctgcaaataattacgaaaccgc

SEQ ID NO: 57 - DNA, 31 bp, Artificial Sequence
  Synthetic Sequence
caatgccaatattagctgaaatgaccaaatc

SEQ ID NO: 58 - DNA, 31 bp, Artificial Sequence
  Synthetic Sequence
ggtcatttcagctaatattggcattgaccgc

SEQ ID NO: 59 - DNA, 34 bp, Artificial Sequence
  Synthetic Sequence
gtctgcgttccagcagcaataaaacatgttttag

SEQ ID NO: 60 - DNA, 34 bp, Artificial Sequence
  Synthetic Sequence
gttttattgctgctggaacgcagacacaaccttc

SEQ ID NO: 61 - DNA, 39 bp, Artificial Sequence
  Synthetic Sequence
ctctaacccgtttggattgataaaaaagcgtccacctcg

SEQ ID NO: 62 - DNA, 41 bp, Artificial Sequence
  Synthetic Sequence
cgcttttttatcaatccaaacgggttagagtggaaacggtc

SEQ ID NO: 63 - DNA, 30 bp, Artificial Sequence
  Synthetic Sequence
ggtttcgtaattattttgaggaacaaagcg

SEQ ID NO: 64 - DNA, 26 bp, Artificial Sequence
  Synthetic Sequence
gttcctcaaaataattacgaaaccgc

SEQ ID NO: 65 - DNA, 32 bp, Artificial Sequence
  Synthetic Sequence
tgccaatattatttgaaatgaccaaatcagcc

SEQ ID NO: 66 - DNA, 40 bp, Artificial Sequence
  Synthetic Sequence
gatttggtcatttcaaataatattggcattgaccgctacc

SEQ ID NO: 67 - DNA, 43 bp, Artificial Sequence
  Synthetic Sequence
ggttgtgtctgcgttccgaaagcaataaaacatgttttagacc

SEQ ID NO: 68 - DNA, 37 bp, Artificial Sequence
  Synthetic Sequence
gttttattgctttcggaacgcagacacaaccttcacg

SEQ ID NO: 69 - DNA, 30 bp, Artificial Sequence
  Synthetic Sequence
tttagaccgcgtccactctaacccgtctgg

SEQ ID NO: 70 - DNA, 30 bp, Artificial Sequence
  Synthetic Sequence
agagtggacgcggtctaaatggtcaagacc

SEQ ID NO: 71 - DNA, 36 bp, Artificial Sequence
  Synthetic Sequence
ttcggatccaactattagcctacattcgagaacagg

SEQ ID NO: 72 - DNA, 40 bp, Artificial Sequence; identical to SEQ ID NO: 52
  Synthetic Sequence

SEQ ID NO: 73 - DNA, 47 bp, Artificial Sequence
  Synthetic Sequence
ctttaagaaggagactcgagatgggacgcttttttatcaatccagac

SEQ ID NO: 74 - DNA, 47 bp, Artificial Sequence
  Synthetic Sequence
gtctggattgataaaaaagcgtcccatctcgagtctccttcttaaag

SEQ ID NO: 75 - DNA, 43 bp, Artificial Sequence
  Synthetic Sequence
ctttaagaaggagactcgagatggggttagagtggaaacggtc

SEQ ID NO: 76 - DNA, 43 bp, Artificial Sequence
  Synthetic Sequence
gaccgtttccactctaaccccatctcgagtctccttcttaaag

SEQ ID NO: 77 - DNA, 32 bp, Artificial Sequence
  Synthetic Sequence
ggatccatgatggcaattacctatgccctgtc

SEQ ID NO: 78 - DNA, 40 bp, Artificial Sequence; identical to SEQ ID NO: 52
  Synthetic Sequence

SEQ ID NO: 79 - DNA, 36 bp, Artificial Sequence
  Synthetic Sequence
ggatccatggaagagttgattagtcatcaatcatct

SEQ ID NO: 80 - DNA, 40 bp, Artificial Sequence; identical to SEQ ID NO: 52
  Synthetic Sequence

SEQ ID NO: 81 - DNA, 37 bp, Artificial Sequence
  Synthetic Sequence
ggtaccatgcgtcatatattcatcataggaagtcgcg

SEQ ID NO: 82 - DNA, 50 bp, Artificial Sequence
  Synthetic Sequence
atattctagaattataggtaccccttattaaagttaaacaaaattatttc

SEQ ID NO: 83 - DNA, 24 bp, Artificial Sequence
  Synthetic Sequence
gctatccgtgagttcatgacttcg

SEQ ID NO: 84 - DNA, 37 bp, Artificial Sequence
  Synthetic Sequence
ctgcagttaactttcatgtaagaacaagtcctcgtac

SEQ ID NO: 85 - DNA, 24 bp, Artificial Sequence
  Synthetic Sequence
cgaagtcatgaactcacggatagc

SEQ ID NO: 86 - DNA, 44 bp, Artificial Sequence
  Synthetic Sequence
ggaggaattcaccttgcgtcatatattcatcataggaagtcgcg

SEQ ID NO: 87 - DNA, 40 bp, Artificial Sequence
  Synthetic Sequence
tctgaattcatgaaacagtcagtttatatcattggttcaa

SEQ ID NO: 88 - DNA, 50 bp, Artificial Sequence
  Synthetic Sequence
ggttgtgtctgcgttccataagcaataaaggtcgtcttgggctgatactg

SEQ ID NO: 89 - DNA, 84 bp, Artificial Sequence
  Synthetic Sequence
ccagattcagaaccctattttttatgtgttggcgtgtcgagtaggcccatttattgcgccatttgtgaagcagattcacaatcg

SEQ ID NO: 90 - DNA, 84 bp, Artificial Sequence
  Synthetic Sequence
cgattgtgaatctgcttcacaaatggcgcaataaatgggcctactcgacacgccaacacataaaaaatagggttctgaatctgg

SEQ ID NO: 91 - DNA, 44 bp, Artificial Sequence
  Synthetic Sequence
caatccagacgggcacgagtggaaactgtctaaatggtcaagac

SEQ ID NO: 92 - DNA, 44 bp, Artificial Sequence
  Synthetic Sequence
gtcttgaccatttagacagtttccactcgtgcccgtctggattg

SEQ ID NO: 93 - DNA, 32 bp, Artificial Sequence; identical to SEQ ID NO: 65
  Synthetic Sequence

SEQ ID NO: 94 - DNA, 40 bp, Artificial Sequence; identical to SEQ ID NO: 66
  Synthetic Sequence

SEQ ID NO: 95 - DNA, 43 bp, Artificial Sequence; identical to SEQ ID NO: 67
  Synthetic Sequence

SEQ ID NO: 96 - DNA, 37 bp, Artificial Sequence; identical to SEQ ID NO: 68
  Synthetic Sequence

SEQ ID NO: 97 - DNA, 37 bp, Artificial Sequence
  Synthetic Sequence
atctgaattcatgcaggatgttttcatcattggtagc

SEQ ID NO: 98 - DNA, 40 bp, Artificial Sequence
  Synthetic Sequence
acactgcagttaatgttcatctaaaaataaagcctcatac

SEQ ID NO: 99 - DNA, 32 bp, Artificial Sequence
  Synthetic Sequence
tctgaattcatgcaagatgttttcattatagg

SEQ ID NO: 100 - DNA, 36 bp, Artificial Sequence
  Synthetic Sequence
acactgcagttaactttcgttcaagaacaagtcctc

SEQ ID NO: 101 - DNA, 38 bp, Artificial Sequence
  Synthetic Sequence
atgaattcatgcaggatgttttcatcattggtagcaga

SEQ ID NO: 102 - DNA, 50 bp, Artificial Sequence
  Synthetic Sequence
atctgcagttaatgttcatctaaaaataaagcctcatactccccaacaat

SEQ ID NO: 103 - DNA, 40 bp, Artificial Sequence; identical to SEQ ID NO: 87
  Synthetic Sequence

SEQ ID NO: 104 - DNA, 44 bp, Artificial Sequence
  Synthetic Sequence
atatctgcaggcatcatacagtaaacacttcctcataatctgac

SEQ ID NO: 105 - DNA, 84 bp, Artificial Sequence
  Synthetic Sequence
ccagattcagaaccctattttttatgtgttggcgtgtcgagtaggcgcttttattgcgccatttgtgaagcagattcacaatcg

SEQ ID NO: 106 - DNA, 84 bp, Artificial Sequence
  Synthetic Sequence
cgattgtgaatctgcttcacaaatggcgcaataaaagcgcctactcgacacgccaacacataaaaaatagggttctgaatctgg

SEQ ID NO: 107 - DNA, 47 bp, Artificial Sequence
  Synthetic Sequence
aagttctgtttcagggcccgaacattaatattttactatccacctac

SEQ ID NO: 108 - DNA, 45 bp, Artificial Sequence
  Synthetic Sequence
atggtctagaaagctttactttctcctgtaaccaaataaggtaac

SEQ ID NO: 109 - DNA, 47 bp, Artificial Sequence
  Synthetic Sequence
aagttctgtttcagggcccgaaggttaatatcttaatggccacctac

SEQ ID NO: 110 - DNA, 50 bp, Artificial Sequence
  Synthetic Sequence
atggtctagaaagctttatctcttattgtaataatttgttgcaatcaacc

SEQ ID NO: 111 - DNA, 47 bp, Artificial Sequence
  Synthetic Sequence
aagttctgtttcagggcccgaaagttaatattttaatgtccacctac

SEQ ID NO: 112 - DNA, 41 bp, Artificial Sequence
  Synthetic Sequence
atggtctagaaagctttattttctcctataaccaaatttag

SEQ ID NO: 113 - DNA, 36 bp, Artificial Sequence
  Synthetic Sequence
aagttctgtttcagggcccgagtaacaagcaaattg

SEQ ID NO: 114 - DNA, 37 bp, Artificial Sequence
  Synthetic Sequence
atggtctagaaagctttaaataaacattaactcaccg

SEQ ID NO: 115 - DNA, 36 bp, Artificial Sequence
  Synthetic Sequence
cttaaatctcttatccattgtacccgcccccaaaac

SEQ ID NO: 116 - DNA, 36 bp, Artificial Sequence
  Synthetic Sequence
gttttgggggcgggtacaatggataagagatttaag

SEQ ID NO: 117 - DNA, 33 bp, Artificial Sequence
  Synthetic Sequence
cgaagtatcttaaatctaccatccattgtcctc

SEQ ID NO: 118 - DNA, 33 bp, Artificial Sequence
  Synthetic Sequence
gaggacaatggatggtagatttaagatacttcg

SEQ ID NO: 119 - DNA, 32 bp, Artificial Sequence
  Synthetic Sequence
gaccttcacgaagtataccaaatctcttatcc

SEQ ID NO: 120 - DNA, 32 bp, Artificial Sequence
  Synthetic Sequence
ggataagagatttggtatacttcgtgaaggtc

SEQ ID NO: 121 - DNA, 35 bp, Artificial Sequence
  Synthetic Sequence
tagatttaggaccttcaccaagtatcttaaatctc

SEQ ID NO: 122 - DNA, 33 bp, Artificial Sequence
  Synthetic Sequence
gagatttaagatacttggtgaaggtcctaaatc

SEQ ID NO: 123 - DNA, 45 bp, Artificial Sequence
  Synthetic Sequence
gcagatgtctattttttcagtgcccaagatgatatatggttagac

SEQ ID NO: 124 - DNA, 45 bp, Artificial Sequence
  Synthetic Sequence
gtctaaccatatatcatcttgggcactgaaaaaatagacatctgc

SEQ ID NO: 125 - DNA, 38 bp, Artificial Sequence
  Synthetic Sequence
cttgatattccaacagaattattccgtcagcacgatgc

SEQ ID NO: 126 - DNA, 38 bp, Artificial Sequence
  Synthetic Sequence
gcatcgtgctgacggaataattctgttggaatatcaag

SEQ ID NO: 127 - DNA, 40 bp, Artificial Sequence
  Synthetic Sequence
caacagaattataccgtcaggccgatgctaacgtgttggg

SEQ ID NO: 128 - DNA, 40 bp, Artificial Sequence
  Synthetic Sequence
cccaacacgttagcatcggcctgacggtataattctgttg

(19) United States
(12) Patent Application Publication
(10) Pub. No.: US 2022/0259629 A1
(43) Pub. Date: Aug. 18, 2022

(54) RHAMNOSE-POLYSACCHARIDES

(71) Applicant: UNIVERSITY OF DUNDEE, DUNDEE (GB)

(72) Inventor: HELGE DORFMUELLER, DUNDEE (GB)

(21) Appl. No.: 17/617,682

(22) PCT Filed: Jun. 12, 2020
(86) PCT No.: PCT/EP2020/066314

§ 371 Date: Dec. 9, 2021

(30) Foreign Application Priority Data
Jun. 13, 2019 (GB) 1908528.1

(51) Int. Cl. C12P 19/04; A61K 39/09; C12P 21/00

(52) U.S. Cl. CPC C12P 19/04; C12Y 204/01288; C12P 21/005; A61K 39/092

(57) ABSTRACT

The present invention relates to a method of synthesizing a rhamnose polysaccharide. The invention also relates to a synthetic streptococcal polysaccharide, a streptococcal glycoconjugate, an immunogenic composition or vaccine comprising the streptococcal polysaccharide or glycoconjugate and the polysaccharide, glycoconjugate, immunogenic composition or vaccine for use in raising an immune response in an animal or for use in treating or preventing a disease, condition or infection with a streptococcal aetiology.

### FIELD

The present invention relates to a method of synthesizing a rhamnose polysaccharide. The invention also relates to a synthetic streptococcal polysaccharide, a streptococcal glycoconjugate, an immunogenic composition or vaccine comprising the streptococcal polysaccharide or glycoconjugate and the polysaccharide, glycoconjugate, immunogenic composition or vaccine for use in raising an immune response in an animal or for use in treating or preventing a disease, condition or infection with a streptococcal aetiology.

### BACKGROUND

The Streptococci genera of bacteria is a group of versatile gram-positive bacteria that infect a wide range of hosts and are responsible for a remarkable number of illnesses.

Streptococcus pyogenes (Group A Streptococcus, GAS) is a human-exclusive pathogenic Gram-positive bacterium that causes a variety of illnesses. A probably underestimated appraisal of the epidemical power of this organism suggests that over 700 million individuals are afflicted per year worldwide, causing diseases as varied as impetigo, pharyngitis, scarlet fever, necrotising fasciitis, meningitis and toxic shock syndrome, amongst other illnesses. Moreover, autoimmune post-infection sequelae, such as acute rheumatic fever, acute glomerulonephritis or rheumatic heart disease can affect individuals that had previously suffered from GAS infections, extending the list of clinical manifestations caused by this pathogen. The Group A Carbohydrate (GAC) is a peptidoglycan-anchored rhamnose-polysaccharide (RhaPS) from Streptococcus pyogenes that is essential to bacterial survival and contributes to Streptococcus pyogenes' ability to infect the human host.

Streptococcus agalactiae (Group B Streptococcus, GBS), is a (pathogenic) commensal bacterium which is carried by 20-40% of all adult humans. 25% of women carry GBS in the vagina, where it normally resides without symptoms. However, in pregnant women, GBS is a recognised cause for preterm delivery, maternal infections, stillbirths and late miscarriages. Despite current prevention strategies, 1 in every 1000 babies born in the UK develop GBS infections. Preterm babies are known to be at particular risk of GBS infection as their immune systems are not as well developed. This results in one baby per week dying in the UK from GBS infection and one baby surviving with long-term disabilities.

Group C Streptococcus (GCS) can cause epidemic pharyngitis and cellulitis clinically indistinguishable from GAS disease in humans. It is also known to cause septicaemia, endocarditis, septic arthritis and necrotizing infections in patients with predisposing conditions such as diabetes, cancer or in elderly patients. In equine animals, GCS is the cause of the highly contagious and serious upper respiratory tract infection known as strangles, which is enzootic in a worldwide distribution.

Group G Streptococcus (GGS) are significant human pathogens that cause cutaneous infections, for example of the human skin. GGS also infect the oropharynx, gastrointestinal regions and female genital tracts. Other infections associated with GGS include several potentially life-threatening infections such as septicaemia, endocarditis, meningitis, peritonitis, pneumonitis, empyema, and septic arthritis.

Antimicrobial options for effectively controlling, treating and preventing GAS infections are becoming more limited. This is due to emerging antibiotic resistance, pandemic development and the spread of hyper virulent strains. There is thus a clear need for the development of a safe and effective vaccine candidate. For a vaccine to be capable of targeting most of the over 120 different GAS serotypes, it will need to be based on a ubiquitous, conserved and essential GAS target. One such target is the GAC, which is not only an essential structural component to the pathogen but is also a virulence determinant.

Current forms of vaccine development are limited to chemical and enzymatic extraction methods from native bacteria as well as chemical conjugation to any acceptor compound, for example a protein or peptide. This is labour-intensive and results in a limited yield and quality of product. There is a clear need for a method of producing a GAS polysaccharide which is less labour-intensive and results in a homogenous, pure and high yield of polysaccharide. The present invention is devised with these issues in mind.

### DESCRIPTION

In its broadest sense, the present disclosure relates to a method of synthesizing a polysaccharide, specifically a rhamnose polysaccharide.

According to a first aspect there is provided a method of synthesizing a rhamnose polysaccharide, the method comprising:

(i) transferring a rhamnose moiety to a hexose monosaccharide, disaccharide, or trisaccharide using a hexose-β-1,4-rhamnosyltransferase, a hexose-α-1,2-rhamnosyltransferase and/or a hexose-α-1,3-rhamnosyltransferase, or an enzymatically active fragment or variant thereof to form a disaccharide, trisaccharide or tetrasaccharide comprising a rhamnose moiety at a non-reducing end of the disaccharide, trisaccharide or tetrasaccharide; and

(ii) generating the rhamnose polysaccharide by extending from the rhamnose moiety at the non-reducing end of the disaccharide, trisaccharide or tetrasaccharide using a heterologous bacterial enzyme Streptococcus pyogenes Group A carbohydrate enzyme C (GacC) and/or Streptococcus pyogenes Group A carbohydrate enzyme G (GacG) or an enzymatically active homologue, variant or fragment thereof.

The bacterial species from which the enzyme GacC and/or the enzyme GacG or an enzymatically active homologue, variant or fragment thereof is derived is heterologous to the bacterial species from which the hexose-β-1,4-rhamnosyltransferase, the hexose-α-1,2-rhamnosyltransferase, the hexose-α-1,3-rhamnosyltransferase or enzymatically active fragment or variant thereof used in step (i) is derived.

The present inventor has discovered for the first time that the Streptococcus pyogenes enzyme GacB, which initiates the synthesis of the GAC rhamnose polysaccharide, is a α-D-GlcNAc-β-1,4-L rhamnosyl-transferase. Entirely surprisingly, the inventor has found that these rhamnose polysaccharides can be synthesized using rhamnosyltransferases from bacterial species different to those from which the GacB is derived. In other words, the inventors have found that rhamnose polysaccharides can be synthesized using rhamnosyltransferases from bacterial species other than S. pyogenes. This is entirely unexpected given that the function of GacB was previously unknown. It is also surprising that enzymes from different species can work together to synthesize a rhamnose polysaccharide.

In some embodiments, step (ii) comprises generating the rhamnose polysaccharide by extending from the rhamnose moiety at the non-reducing end of the disaccharide, trisaccharide or tetrasaccharide using the heterologous bacterial enzyme GacC or an enzymatically active homologue, variant or fragment thereof.

Polysaccharide is a known term of the art used to denote a molecule comprising a plurality of identical or different monosaccharides, typically more than four monosaccharides. The term rhamnose polysaccharide, as used herein, will thus be understood to refer to a molecule comprising a plurality, typically more than four, rhamnose moieties, optionally attached to one or more other monosaccharide moieties. Conveniently, the rhamnose polysaccharide may be a single straight chain of repeating units comprising rhamnose, bound to each other by alpha 1,3, or alpha 1,2 bonds. Each repeating unit may consist only of rhamnose, or each repeating unit may comprise rhamnose and one or more different monosaccharides. An exemplary repeating unit which comprises rhamnose is a rhamnose-galactose disaccharide repeating unit. Each/any repeating unit and/or rhamnose moiety may or may not include any side-group. In one embodiment no side groups are present and in another embodiment one or more side groups, such as a sugar, with or without additional modifications, such as glycerol-phosphate; or phosphate, may be present.

In embodiments, the method is performed in a bacterium.

In such embodiments, the method will be understood to be a microbiological method. Embodiments other than those carried out in a bacterium will be understood to be in vitro methods. By “bacterium”, this will be understood to refer to a bacterial cell. It will be appreciated that the invention also encompasses the method being performed in bacteria. Such microbiological methods are ideal for the production of large and homogenous quantities of a particular product, in this instance a rhamnose polysaccharide.

The rhamnose polysaccharide produced by the method will be understood to be a synthetic rhamnose polysaccharide. A synthetic rhamnose polysaccharide, as the skilled person will appreciate, will be understood to refer to a rhamnose polysaccharide, which is not the result of a naturally occurring process. This is because the method of the first aspect uses enzymes, the combination of which is not naturally occurring. In one embodiment, the bacterium is a Streptococcus species other than Streptococcus pyogenes, Escherichia species, such as E. coli, or a Shigella species, such as Shigella dysenteriae or Shigella flexneri.

Typically, the rhamnose polysaccharide produced by the method is a streptococcal polysaccharide. For example, the polysaccharide may comprise a polysaccharide or a fragment or variant thereof selected from the group consisting of a Group A, Group B, Group C and Group G carbohydrate.

By rhamnose moiety, this will be understood to refer to a rhamnose monosaccharide or a derivative thereof. It will be appreciated that derivatives of rhamnose refer to a rhamnose monosaccharide(s) which has been modified by the addition or replacement of one or more groups or elements in the rhamnose monosaccharide, provided that at least one carbon of the rhamnose monosaccharide is still capable of forming a glycosidic bond with at least one other rhamnose monosaccharide or rhamnose moiety. Derivatives of rhamnose may encompass acetyl or methyl forms of rhamnose, amino-rhamnose, carboxylethyl-rhamnose, halogenated rhamnose and rhamnose phosphate. Unless context otherwise dictates, herein after reference will generally be made to a rhamnose moiety, but this should not be construed as limiting. Halogenated rhamnose will be understood to refer to a rhamnose monosaccharide wherein one or more groups of the rhamnose, for example one or more OH groups is replaced with a halogen, for example fluoride or chloride to form a fluorinated or chlorinated rhamnose, respectively.

Amino-rhamnose will be understood to refer to a rhamnose monosaccharide where one or more groups of the rhamnose is replaced by an amine group.

An example acetyl-rhamnose may comprise 2-O-acetyl-α-L-rhamnose, while an example methyl-rhamnose may comprise 3-O-methyl-L-rhamnose. Another exemplary derivative of rhamnose may comprise carboxylethyl-rhamnose, for example 4-O-(1-carboxyethyl)-L-rhamnose.

By enzymatically active fragment or variant, we include that the sequence of the relevant enzyme can vary from the naturally occurring sequence with the proviso that the fragment or variant substantially retains the enzymatic activity of the enzyme. By retain the enzymatic activity of the enzyme it is meant that the fragment and/or variant retains at least a portion of the enzymatic activity as compared to the native enzyme. Typically, the fragment and/or variant retains at least 50%, such as 60%, 70%, 80%, 90%, 95%, 97%, 98% or 99% activity.

In some instances, the fragment and/or variant may have a greater enzymatic activity than the native enzyme. In some embodiments, the fragment and/or variant may display an increase in another physiological feature as compared to the native enzyme. For example, the fragment and/or variant may possess a greater half-life in vitro and/or in vivo, as compared to the native enzyme. The test for determining the half-life of an enzyme, or a fragment or variant thereof, will be known to the skilled person. Briefly, an in vitro test may involve incubating the enzyme at a particular temperature and pH for different time periods. At the end of each time period, the activity of the enzyme, or fragment or variant thereof, can be measured using an enzymatic assay, which is well known to the skilled person.

The enzyme GacC, as used herein, will be understood to refer to the Streptococcus pyogenes Group A carbohydrate enzyme C (UniProtKB—Q9A0G4 (Q9A0G4_STRP1)). An exemplary amino acid sequence encoding GacC is provided by SEQ ID NO:1.

The enzyme GacG, as used herein, will be understood to refer to the Streptococcus pyogenes Group A carbohydrate enzyme G (UniProtKB—Q9A0G0 (Q9A0G0_STRP1)). In some embodiments, the enzyme GacG comprises or consists of SEQ ID NO:2, or an enzymatically active fragment or variant thereof.

GacG (or an enzymatically active homologue, variant or fragment thereof) is used instead of or in addition to GacC in the method of the invention. GacC is a rhamnose-1,3 α rhamnosyltransferase, while GacG is a predicted dual function glycosyltransferase, that synthesizes the repeating unit for the GAC (alpha 1,3-alpha1,2).

“Homologue” may encompass enzymes which exhibit at least about 20%, 30%, 40%, 50%, 60%, 65%, 70%, 75%, 80%, 85%, 90%, 91%, 92%, 93%, 94%, 95%, 96%, 97%, 98% or 99% identity to a GacC or GacG amino acid sequence.

In some embodiments, the enzymatically active homologue is a homologue of GacC.

The degree of (or percentage) “homology” between two or more amino acid sequences may be calculated by aligning the sequences and determining the number of aligned residues which are identical and adding this to the number of conservative amino acid substitutions. The combined total is then divided by the total number of residues compared and the resulting figure is multiplied by 100—this yields the percentage homology between aligned sequences.

Typically, a homologue of GacC or GacG encompasses an enzyme which substantially retains the enzymatic activity of GacC or GacG.

In some embodiments, the homologue of GacC comprises or consists of rfbG. RfbG is an alpha-1-3 rhamnosyltransferase derived from Shigella flexneri which has 30% identity to GacC. Thus, in the context of the present invention, rfbG is an enzymatically active homologue of GacC. In some embodiments, rfbG comprises or consists of SEQ ID NO: 3. RfbG may be identified using the UniProtKB—A0A2D0WWB9 (A0A2D0WWB9_9ENTR).

The homologue of GacC or GacG may comprise or consist of rfbG, an enzyme derived from a Lancefield group species other than S. pyogenes and/or from a non-Lancefield group Streptococcus species other than S. pneumoniae.

In some embodiments, the homologue of GacC or GacG is an enzyme derived from a Lancefield group species other than S. pyogenes and/or from a non-Lancefield group Streptococcus species other than S. pneumoniae.

As the skilled person will be aware, the Lancefield group of bacteria refers to a group of different bacterial species, primarily Streptococcus species, which are catalase-negative and coagulase-negative. The grouping is based on the carbohydrate composition of the cell wall antigens.

Lancefield group bacteria include:
Group A—Streptococcus pyogenes, Streptococcus dysgalactiae subsp. equisimilis
Group B—Streptococcus agalactiae
Group C—Streptococcus equisimilis, Streptococcus equi, Streptococcus zooepidemicus, Streptococcus dysgalactiae, Streptococcus dysgalactiae subsp. equisimilis
Group D—Enterococcus faecalis, Enterococcus faecium, Enterococcus durans and Streptococcus bovis
Group E—Enterococci
Group F, G & L—Streptococcus anginosus, Streptococcus dysgalactiae subsp. equisimilis
Group H—Streptococcus sanguis
Group K—Streptococcus salivarius
Group L—Streptococcus dysgalactiae
Group M & O—Streptococcus mitior
Group N—Lactococcus lactis
Group R & S—Streptococcus suis

The non-Lancefield group Streptococcus species may comprise Streptococcus mutans or S. uberis. In some embodiments, the non-Lancefield group Streptococcus species may comprise or consist of S. mutans.

The enzymatically active homologue of GacC or GacG may be selected from a homologue from the Streptococcus Group B, Group C, Group G, S. mutans, S. uberis or an enzymatically active fragment or variant thereof.

In some embodiments, the enzymatically active homologue of GacC or GacG may be selected from a homologue from the Streptococcus Group B, Group C, Group G, S. mutans, or an enzymatically active fragment or variant thereof.

In some embodiments, the enzymatically active homologue of GacC is selected from a homologue of GacC from the Streptococcus Group B, Group C, Group G, S. mutans, S. uberis or an enzymatically active fragment or variant thereof. The skilled person will be aware of Streptococcal homologues to GacC. For example, the Group B homologue of GacC may be GbcC (UniProtKB—Q8DYQ2 (Q8DYQ2_STRA5)). The Group C homologue of GacC may be GccC (UniProtKB—M4YWQ3 (M4YWQ3_STREQ)). The Group G homologue of GacC may be GgcC (UniProtKB—C5WFT8 (C5WFT8_STRDG)), while the S. mutans homologue of GacC may be SccC (UniProtKB—A0A0E2EN43 (A0A0E2EN43_STRMG). The S. uberis homologue of GacC may be SucC (UniProtKB—B9DU25 (B9DU25_STRU0)).

The amino acid sequence of GbcC may comprise or consist of SEQ ID NO:4. The amino acid sequence of GccC may comprise of consist of SEQ ID NO:5, while the amino acid sequence of GgcC may comprise of consist of SEQ ID NO:6. In some embodiments, SccC comprises or consists of SEQ ID NO:7. The amino acid sequence of SucC may comprise or consist of SEQ ID NO:8.

In some embodiments, the enzymatically active homologue of GacG is selected from a homologue of GacG from the Streptococcus Group C, Group G, S. mutans, S. uberis or an enzymatically active fragment or variant thereof. Suitable enzymatically active homologues of GacG include, but are not limited to, the Group C homologue of GacG, GccG, the Group G homologue of GacG, GgcG, the S. uberis homologue of GacG, SucG, and the S. mutans homologue of GacG, SccG.

In some embodiments, GccG comprises and consists of SEQ ID NO:9. In some embodiments, GccG comprises or consists of two proteins. The two proteins may comprise or consist SEQ ID Nos 10 and 11.

GgcG may comprise or consist of two proteins. The two proteins may have the UniProtKBs C5WFU2 (C5WFU2_STRDG) and C5WFU3 (C5WFU3_STRDG), respectively. In some embodiments, GgcG may comprise or consist of SEQ ID Nos 12 and 13.

SucG may comprise or consist of the amino acid sequence identified by the UniProtKB—B9DU29 (B9DU29_STRU0). For example, SucG may comprise or consist of the amino acid sequence SEQ ID NO:14.

SccG may comprise or consist of the amino acid sequence identified by the UniProtKB—082878 (082878_STRMG). In some embodiments, SccG comprises or consists of the amino acid sequence SEQ ID NO:15.

The enzymatically active homologue of GacC or GacG may be selected from a homologue from, S. mutans, S. uberis or a fragment or variant thereof.

In some embodiments, step (ii) comprises generating the rhamnose polysaccharide by extending from the rhamnose moiety at the non-reducing end of the disaccharide, trisaccharide or tetrasaccharide using an enzymatically active homologue of GacC and/or GacG from S. mutans, or an enzymatically active variant or fragment thereof.

The invention also encompasses nucleic acid sequences encoding the enzymes (and/or enzymatically active fragments, variants or homologues) of the present invention.

As used herein, when an enzyme is “derived from” a particular bacterial species, this means that the enzyme is naturally occurring in the particular bacterial species. In the context of the present invention, an enzyme “derived from” a particular bacterial species may include an enzyme endogenous to the bacterium in which the method may be performed, an enzyme or a nucleic acid encoding the enzyme isolated from the particular bacterial species, or variants or fragments thereof. In embodiments where the method is performed in a bacterium, the enzyme or nucleic acid encoding the enzyme isolated from the particular bacterial species may be transferred into the bacterium in which the method is performed.

In embodiments where the method is performed in a bacterium, the enzyme(s) of step (i) and/or the enzymes(s) of step (ii) may be overexpressed in the bacterium. By “overexpressed”, this will be understood to refer to a level of expression of the enzyme higher than that which would be observed for the naturally occurring enzyme when endogenously expressed in its native bacterium. Various techniques for overexpression are known to those skilled in the art. Further information regarding overexpression techniques may be found in Current Protocols in Molecular Biology (2019) which is incorporated herein by reference.

In the context of the present invention, heterologous is used to refer to different. A heterologous bacterial species will be understood to mean a bacterial species different to another, or bacterial genera different to another bacterial genera.

It will be appreciated that in the context of the present invention, heterologous does not encompass a bacterial strain being different to another bacterial strain (i.e., two strains, for example, of S. mutans).

By “variants” of an enzyme we include insertions, deletions and substitutions of the amino acid sequence, either conservative or non-conservative wherein the physio-chemical properties of the respective amino acid(s) are not substantially changed (for example, conservative substitutions such as Gly, Ala; Val, lie, Leu; Asp, Glu; Asn, Gln; Ser, Thr; Lys, Arg; and Phe, Tyr). The skilled person will appreciate that such conservative substitutions should not affect the functionality of the respective enzyme. Moreover, small deletions within non-functional regions of the enzyme can also be tolerated and hence are considered “variants” for the purpose of the present invention. “Variants” also include recombinant enzyme proteins in which the amino acids have been post-translationally modified, by for example, glycosylation, or disulphide bond formation. The experimental procedures described herein can be readily adopted by the skilled person to determine whether a “variant” can still function as an enzyme.

It is preferred if the variant has an amino acid sequence which has at least 75%, yet still more preferably at least 80%, in further preference at least 85%, in still further preference at least 90% and most preferably at least 95%, 97%, 98% or 99% identity with the “naturally occurring” amino acid sequence of the enzyme.

It will be appreciated that variants also encompass variants of the nucleic acid sequence encoding the enzyme. In particular, we include variants of the nucleotide sequence where such changes do not substantially alter the enzymatic activity of the enzyme which it encodes.

A skilled person would know that such sequences can be altered without the loss of enzymatic activity. In particular, single changes in the nucleotide sequence may not result in an altered amino acid sequence following expression of the sequence.

In some embodiments, the method is performed in a bacterium species heterologous to the bacterium species or genera from which the enzyme GacC and/or GacG or an enzymatically active homologue, variant or fragment thereof is derived. In some embodiments, the method is performed in a gram-positive bacterium. The method may be performed in a gram-negative bacterium. For example, the method may be performed in a gram-negative bacterium such as E. coli or Campylobacter species. Other suitable gram-negative bacteria will be known to the skilled person. In embodiments, the bacterium species may be heterologous to the bacterium species or genera from which the hexose-β-1,4-rhamnosyltransferase, hexose-α-1,2-rhamnosyltransferase or hexose-α-1,3-rhamnosyltransferase is derived.

In some embodiments, the method is performed in E. coli.

Step ii) of the method may comprise using one or more additional enzymes from the Gac cluster of bacterial enzymes, or one or more enzymatically active homologue(s), variant(s) or fragment(s) thereof.

As the skilled person will appreciate, GacB is one of a number of enzymes encoded by one gene cluster in S. pyogenes. This gene cluster, which may otherwise be referred to as the Gac gene cluster, (gacA-gacL, MGAS5005_Spy_0602-0613) is understood to encode 12 different enzymes, as defined by van Sorge et al., 2014. The 12 enzymes are GacA, GacB, GacC, GacD, GacE, GacF, GacG, GacH, Gacl, GacJ, GacK and GacL. Thus, step ii) of the method may further comprise using one or more additional enzymes from the Gac cluster of bacterial enzymes, or one or more enzymatically active homologue(s), variant(s) or fragment(s) thereof. Thus, In some embodiments, step ii) of the method comprises using one or more additional enzymes selected from GacA, GacC, GacD, GacE, GacF, GacG, GacH, Gacl, GacJ, GacK, GacL or one or more enzymatically active homologue(s), variant(s) or fragment(s) thereof.

In some embodiments, step ii) of the method further comprises using one or more enzymatically active homologue(s), or enzymatically active variant(s) or fragment(s) thereof, of one or more of GacA, GacC, GacD, GacE, GacF, GacG, GacH, Gacl, GacJ, GacK, GacL.

The one or more enzymatically active homologue(s) may be derived from S. mutans and/or S. uberis.

In some embodiments, the one or more enzymatically active homologue(s) is derived from S. mutans.

Step ii) may further comprise using the enzyme GacA or an enzymatically active homologue, fragment or variant thereof. In some embodiments, step ii) may comprise using the enzymes GacC and GacG, or one or more enzymatically active homologue(s), variant(s) or fragment(s) thereof.

In some embodiments, step ii) comprises using the enzymes GacC, GacA and GacG, or one or more enzymatically active homologues, variants or fragments thereof. Step ii) may further comprise using the enzymes GacD, GacE, and GacF or one or more enzymatically active homologue(s), fragment(s) or variant(s) thereof.

Step ii) may comprise using the enzymes GacC, GacA, GacG, GacD, GacE, and Gac F or one or more enzymatically active homologue(s), fragment(s) or variant(s) thereof.

In some embodiments, step ii) comprises using the enzymes GacA, GacC, GacD, GacE, GacF, GacG, GacH, Gacl, GacJ, GacK and GacL, or one or more enzymatically active homologue(s), variant(s) or fragment(s) thereof.

Step ii) may comprise using the enzymatically active homologues from S. mutans and/or S. uberis of GacA, GacC, GacD, GacE, GacF, GacG and GacH.

In some embodiments, step ii) comprises using the enzymatically active homologues from S. mutans of GacA, GacC, GacD, GacE, GacF, GacG and GacH.

GacA may comprise or consist of SEQ ID NO:16. Without wishing to be bound by theory, GacA is believed to function to synthesize the rhamnose moieties required for the generation of the rhamnose polysaccharide. GacG is believed to be involved in the generation of the rhamnose polysaccharide by extending from the rhamnose moiety at the reducing end.

GacD and GacE may function to form an ATP-dependent ABC transporter. As the skilled person will appreciate, an ATP-dependent ABC transporter translocates substrates across membranes. Thus, without wishing to be bound by theory, GacD and GacE may assist in transporting the rhamnose polysaccharide across the bacterial membrane such that it can then be presented on the bacterial cell wall.

GacH may comprise or consist of SEQ ID NO:17. GacH can also be identified using UniProtKB—J7M7C2 (J7M7C2_STRP1).

In some embodiments, step ii) further comprises using the enzymes GacH, Gacl, GacJ, GacK and GacL, or one or more enzymatically active homologue(s), variant(s) or fragment(s) thereof.

It is thought that Gacl and/or GacJ may enhance the catalytic efficiency of the method of synthesizing the rhamnose polysaccharide.

Enzymatically active homologues of GacA may be selected from a homologue of GacA from the Streptococcus Group B, Group C, Group G, S. mutans, S. uberis or an enzymatically active fragment or variant thereof. For example, the Streptococcus Group B homologue of GacA is RmlD. The Streptococcus Group C homologue of GacA is RmlD, as is the Streptococcus Group G homologue of GacA.

The Streptococcus Group B homologue of GacA, RmlD may have the UniProtKB—A0A0E1EP43 (A0A0E1EP43_STRAG). In some embodiments, the Streptococcus Group B homologue of GacA, RmlD comprises or consists of SEQ ID NO:18.

The Streptococcus Group C homologue of GacA, RmlD may have the UniProtKB—K4Q921 (K4Q921_STREQ). In some embodiments, the Streptococcus Group C homologue of GacA, RmlD comprises or consists of SEQ ID NO:19.

The Streptococcus Group G homologue of GacA, RmlD may have the UniProt—KB AOA2X3AIL5 (AOA2X3AIL5_STRDY). The Streptococcus Group G homologue of GacA may comprise or consist of SEQ ID NO:20.

The S. mutans homologue of GacA may be identified using the UniProtKB—033664 (033664_STRMG). In some embodiments, the S. mutans homologue of GacA may comprise or consist of SEQ ID NO:21.

The S. uberis homologue of GacA may be identified using the UniProtKB—B9DU23 (B9DU23_STRU0). In some embodiments, the S. uberis homologue of GacA may comprise or consist of SEQ ID NO:22.

Enzymatically active homologues of GacD, GacE and/or GacF, may be selected from homologues from the Streptococcus Group C, Group G, S. mutans, S. uberis or an enzymatically active fragment or variant thereof. Suitable homologues of GacD include, but are not limited to, the Streptococcus Group C enzyme GccD, the Streptococcus Group G enzyme GgcD and the S. mutans enzyme SccD. Suitable homologues of GacE include, but are not limited to, the Streptococcus Group C enzyme GccE, the Streptococcus Group G enzyme GgcE and the S. mutans enzyme SccE. Suitable homologues of GacF include, but are not limited to, the Streptococcus Group C enzyme GccF, the Streptococcus Group G enzyme GgcF, the S. mutans enzyme SccF and the S. uberis enzyme SucF.

In some embodiments, GccD comprises or consists of the amino acid sequence SEQ ID NO:23. GccE may be identified using the UniProtKB—AOA380KIL0 (AOA380KIL0_STREQ).

In some embodiments, GccE comprises or consists of the amino acid sequence SEQ ID NO:24. GccF may be identified using the UniProtKB—A0A3S4QIR3 (A0A3S4QIR3_STREQ). Optionally, GccF comprises or consists of SEQ ID NO:25.

In some embodiments, GgcD comprises or consists of the amino acid sequence SEQ ID NO:26. GgcD may be identified using the UniProtKB—C5WFT9 (C5WFT9_STRDG).

In some embodiments, GgcE is identified by the UniProtKB—M4YXS7 (M4YXS7_STREQ). Optionally, GgcE comprises or consists of SEQ ID NO:27. GgcF may be identified by the UniProtKB—C5WFU1 (C5WFU1_STRDG). In some embodiments, GgcF comprises or consists of SEQ ID NO:28.

SccD may comprise or consist of SEQ ID NO:29. Optionally, SccD is identified using the UniProtKB—I6L8Z4 (I6L8Z4_STRMU).

SccE may comprise or consist of SEQ ID NO:30. Optionally, SccE is identified using the UniProtKB—I6L8X8 (I6L8X8_STRMU).

SccF may be identified using the UniProtKB—082877 (082877_STRMG). Optionally, SccF comprises or consists of SEQ ID NO:31.

SucD may be identified using the UniProtKB—B9DU26 (B9DU26_STRU0). In some embodiments, SucD comprises or consists of SEQ ID NO:32.

SucE may be identified using the UniProtKB—B9DU27 (B9DU27_STRU0). In some embodiments, SucE comprises or consists of SEQ ID NO:33.

SucF may be identified using the UniProtKB—B9DU28 (B9DU28_STRU0). In some embodiments, SucF comprises or consists of the amino acid sequence SEQ ID NO:34.

An enzymatically active homologue of GacH may comprise or consist of the S. mutans enzyme SccH, or an enzymatically active fragment or variant thereof. The enzyme SccH may be identified using the UniProtKB—Q8DUS0 (Q8DUS0_STRMU).

In some embodiments, SccH comprises or consists of SEQ ID NO:35.

In some embodiments, the hexose-β-1,4-rhamnosyltransferase is not a N-acetylglucosamine (GlcNAc)-β-1,4-rhamnosyltransferase. In some embodiments, the hexose-β-1,4-rhamnosyltransferase is not GacB.

By “hexose-β-1,4-rhamnosyltransferase”, this will be understood to be an enzyme capable of transferring a rhamnose moiety to a hexose such that a β-1,4 linkage is formed between the hexose and the rhamnose moiety. Once the rhamnose moiety is transferred, it will be understood that the hexose is at the reducing end and the rhamnose moiety is at the non-reducing end, i.e., the end from which is extended from to generate the rhamnose polysaccharide.

The hexose-β-1,4-rhamnosyltransferase may comprise or consist of an allose-β-1,4-rhamnosyltransferase, an altrose-β-1,4-rhamnosyltransferase, a glucose-β-1,4-rhamnosyltransferase, a mannose-β-1,4-rhamnosyltransferase, a xylose-β-1,4-rhamnosyltransferase, a idose-β-1,4-rhamnosyltransferase, a galactose-β-1,4-rhamnosyltransferase a talose-β-1,4-rhamnosyltransferase, a diacetylbacillosamine-β-1,4-rhamnosyltransferase or an enzymatically active fragment or variant thereof.

In some embodiments, the hexose-β-1,4-rhamnosyltransferase comprises a glucose (Glc)-β-1,4-rhamnosyltransferase or an enzymatically active fragment or variant thereof. As the skilled person will appreciate, a glucose (Glc)-β-1,4-rhamnosyltransferase is an enzyme capable of transferring a rhamnose moiety to a glucose, thereby forming a β-1,4 linkage between the glucose and the rhamnose moiety. The hexose-β-1,4-rhamnosyltransferase may comprise a WchF enzyme, or an enzymatically active fragment or variant thereof. The WchF enzyme will be understood to be derived from S. pneumoniae and is a glucose (Glc)-β-1,4-rhamnosyltransferase.

In some embodiments, the WchF enzyme comprises SEQ ID NO:36, or an enzymatically active fragment or variant thereof.

The enzymatically active fragment or variant of WchF may have at least 30% amino acid sequence identity to the WchF enzyme.

In some embodiments, the enzymatically active fragment or variant of WchF has at least 80%, at least 85%, at least 90%, at least 95%, at least 97% or at least 99% amino acid identity to the WchF enzyme. For example, homologues of WchF from S. mitis, S. oralis, S. pseudopneumoniae and S. perosis share 87%, 93%, 87% and 81% amino acid identity to WchF, respectively. In the context of the present invention, these particular homologues will thus be understood to be enzymatically active variants of WchF.

The hexose-α-1,2-rhamnosyltransferase may comprise or consist of an allose-α-1,2-rhamnosyltransferase, an altrose-α-1,2-rhamnosyltransferase, a glucose-α-1,2-rhamnosyltransferase, a mannose-α-1,2-rhamnosyltransferase, a xylose-α-1,2-rhamnosyltransferase, a idose-α-1,2-rhamnosyltransferase, a-galactose α-1,2-rhamnosyltransferase a talose-α-1,2-rhamnosyltransferase, a diacetylbacillosamine-α-1,2-rhamnosyltransferase, a GlcNAc-α-1,2-rhamnosyltransferase or an enzymatically active fragment or variant thereof.

In some embodiments, the hexose-α-1,2-rhamnosyltransferase comprises or consists of a galactose-α-1,2-rhamnosyltransferase or an enzymatically active fragment or variant thereof. The hexose-α-1,2-rhamnosyltransferase may comprise a WbbR enzyme, or an enzymatically active fragment or variant thereof. As the skilled person will appreciate, the WbbR enzyme (WP_001045977.1—UniProtKB—Q32EG0 (Q32EG0_SHIDS) is derived from Shigella dysenterica and is a galactose-α-1,2-rhamnosyltransferase.

The WbbR enzyme may comprise or consist of SEQ ID NO:37.

The hexose-α-1,3-rhamnosyltransferase may comprise or consist of an allose-α-1,3-rhamnosyltransferase, an altrose-α-1,3-rhamnosyltransferase, a glucose-α-1,3-rhamnosyltransferase, a mannose-α-1,3-rhamnosyltransferase, a xylose-α-1,3-rhamnosyltransferase, a idose-α-1,3-rhamnosyltransferase, a galactose-α-1,3-rhamnosyltransferase a talose-α-1,3-rhamnosyltransferase, a diacetylbacillosamine-α-1,3-rhamnosyltransferase, a GlcNAc-α-1,3-rhamnosyltransferase or an enzymatically active fragment or variant thereof

In some embodiments, the hexose-α-1,3-rhamnosyltransferase comprises or consists of a GlcNAc-α-1,3-rhamnosyltransferase, a diNAcBac-α-1,3-rhamnosyltransferase, a Glc-α-1,3-rhamnosyltransferase, a galactose-α-1,3-rhamnosyltransferase or a fragment or variant thereof. The hexose-α-1,3-rhamnosyltransferase may comprise or consist of a GlcNAc-α-1,3-rhamnosyltransferase or a galactose-α-1,3-rhamnosyltransferase or an enzymatically active fragment or variant thereof.

The GlcNAc-α-1,3-rhamnosyltransferase may comprise a WbbL enzyme, or an enzymatically active fragment or variant thereof. The WbbL enzyme is derived from E. coli. The WbbL enzyme may comprise or consist of SEQ ID NO:38, or an enzymatically active fragment or variant thereof.

The enzymatically active fragment or variant of WbbL may have at least 20% or at least 25% amino acid sequence identity to the WchF enzyme. For example, a homologous enzyme of WbbL having 27% amino acid identity to WbbL has been identified in Mycobacterium tuberculosis, also known as WbbL. Thus, in the context of the present invention, this homologue will be understood to be an enzymatically active variant of WbbL. This homologous enzyme to WbbL, derived from Mycobacterium tuberculosis may comprise or consist of SEQ ID NO: 39. Another suitable homologue of WbbL comprises or consists of the enzyme rfbF, derived from Shigella flexneri. RfbF may comprise or consist of SEQ ID NO:40. RfbF can be identified using the UniProtKB—A0A2Y2Z310 (A0A2Y2Z310_SHIFL).

The galactose-α-1,3-rhamnosyltransferase may comprise a WsaD enzyme, or an enzymatically active fragment or variant thereof. The WsaD enzyme is derived from Geobacillus stearothermophilus. In some embodiments, the WsaD enzyme comprises or consists of SEQ ID NO:41.

Enzymatically active fragments or variants of WsaD may be derived from other Bacilli strains, for example Brevibacillus species and Paenibacillus species. The enzymatically active fragments or variants of WsaD may have at least 20%, 30%, at least 40%, at least 50%, at least 60%, at least 70%, at least 80%, at least 85%, at least 90%, at least 95%, at least 97%, at least 98% or at least 99% amino acid identity to WsaD.

The inventors have surprisingly found that a chimera of the hexose-β-1,4-rhamnosyltransferase, the hexose-α-1,2-rhamnosyltransferase the hexose-α-1,3-rhamnosyltransferase, or an enzymatically active fragment or variant with GacB or an enzymatically active variant, fragment or homologue thereof is capable of transferring the rhamnose moiety to a hexose monosaccharide, disaccharide or trisaccharide. Thus, in some embodiments, transferring a rhamnose moiety to a hexose monosaccharide, disaccharide or trisaccharide uses a GacB/hexose-β-1,4-rhamnosyltransferase, hexose-α-1,2-rhamnosyltransferase, hexose-α-1,3-rhamnosyltransferase or enzymatically active fragments or variants thereof chimera. It will be appreciated that in such embodiments the hexose-β-1,4-rhamnosyltransferase is not GacB.

The chimera may comprise at least the C terminus region of GacB linked to the N terminus region of the hexose-β-1,4-rhamnosyltransferase, the hexose-α-1,2-rhamnosyltransferase the hexose-α-1,3-rhamnosyltransferase, or an enzymatically active fragment or variant thereof. In some embodiments, the chimera comprises the C terminus region of GacB linked to the N terminus region of WchF.

In some embodiments, the chimera comprises the full amino acid sequence of GacB except for the initial 50, 100, 150, 160, 170, 180, 190 or 200 amino acids, which are replaced with the corresponding hexose-β-1,4-rhamnosyltransferase, hexose-α-1,2-rhamnosyltransferase hexose-α-1,3-rhamnosyltransferase, or an enzymatically active fragment or variant thereof amino acids. An example chimera may comprise the amino acid sequence of GacB except that the first 178 amino acids of GacB are replaced with the corresponding WchF amino acids (1-186 amino acids).

The hexose monosaccharide, disaccharide or trisaccharide to which the rhamnose moiety is transferred can be any hexose. In embodiments, the hexose monosaccharide is not a rhamnose moiety.

In embodiments wherein the rhamnose moiety is transferred to a hexose disaccharide or trisaccharide, the monosaccharides of the di or trisaccharide may be the same or different to each other. For example, the disaccharide may comprise two galactose monosaccharides. Alternatively, the disaccharide may comprise a GlcNAc and a galactose. The GlcNAc may be at the reducing end of the disaccharide, and the galactose at the non-reducing end.

The disaccharide may comprise one rhamnose moiety. The trisaccharide may comprise one or two rhamnose moieties.

In some embodiments, the monosaccharide at the reducing end of the hexose monosaccharide, disaccharide or trisaccharide to which the rhamnose moiety is transferred (so the hexose monosaccharide or first monosaccharide of the disaccharide or trisaccharide) is a glucose or a glucose derivative.

In the context of the present invention, glucose derivative will be understood to refer to GlcNAc or diNAcBac. In some embodiments, the hexose monosaccharide, disaccharide or trisaccharide does not comprise GlcNAc.

It will be appreciated that the monosaccharide at the non-reducing end of the hexose monosaccharide, disaccharide or trisaccharide determines the specificity of the rhamnosyltransferase. This is because the rhamnosyltransferase transfers the rhamnose moiety to the monosaccharide at the non-reducing end of the hexose monosaccharide, disaccharide or trisaccharide. Thus, when the monosaccharide at the non-reducing end is galactose, the hexose rhamnosyltransferase will be a galactose rhamnosyltransferase.

The disaccharide or trisaccharide may comprise a rhamnose moiety at its non-reducing end.

An exemplary disaccharide may comprise a glucose at the reducing end linked to a rhamnose moiety at the non-reducing end. Other exemplary disaccharides include, but are not limited to, a diNAcBac at the reducing end linked to a rhamnose moiety at the non-reducing end, or a galactose at the reducing end linked to a rhamnose moiety at the non-reducing end.

Exemplary trisaccharides include, but are not limited to, a glucose at the reducing end linked to a hexose which is linked to a rhamnose moiety at the non-reducing end, a diNAcBac at the reducing end linked to a hexose which is linked to a rhamnose moiety at the non-reducing end, or a GlcNAc at the reducing end linked to a hexose which is linked to a rhamnose moiety at the non-reducing end. Optionally, the hexose of the trisaccharide may be a rhamnose moiety or a galactose.

When reference is made to a “link” between hexoses, this will be understood to refer to a glycosidic bond. In the di or trisaccharide, the glycosidic bond between two hexoses in the di- or trisaccharide may be an alpha (α) or a beta (β) glycosidic bond. The alpha bond may be an alpha 1,3 or an alpha 1,2 bond. The beta bond may be a beta 1,4 bond.

The features of the hexose monosaccharide, disaccharide and trisaccharide as described herein are also applicable to the hexose monosaccharide, disaccharide and trisaccharide, as appropriate of the streptococcal polysaccharide of the invention.

Further examples of monosaccharides, disaccharides and trisaccharides to which the rhamnose moiety can be transferred in step i) of the method and/or which comprise or consist of the hexose monosaccharide, disaccharide or trisaccharide of the streptococcal polysaccharide of the invention are provided in Example 2.

In embodiments wherein step (i) comprises transferring a rhamnose moiety to a hexose disaccharide or trisaccharide, the method may further comprise forming the hexose disaccharide or trisaccharide. The hexose disaccharide or trisaccharide may be formed using a hexosyltransferase, i.e., an enzyme capable of transferring a hexose to another hexose. For the hexose trisaccharide, if each monosaccharide of the trisaccharide is the same (for example the trisaccharide is formed of three glucoses), then one hexosyltransferase can be used to transfer each hexose to the other to form the trisaccharide. However, in embodiments where the hexose trisaccharide is formed of at least two different hexoses, then two different hexosyltransferases will be required to form the hexose trisaccharide.

When the method further comprises forming the hexose disaccharide, the hexose disaccharide may be formed using a hexose-α-1,3-hexosyltransferase or an enzymatically active fragment or variant thereof. A hexose-α-1,3-hexosyltransferase will be understood to refer to an enzyme which is capable of transferring a hexose to another hexose to form a α-1,3 bond. In the context of the present invention, bond may otherwise be used to refer to linkage. In some embodiments, the hexose disaccharide is formed using a hexose-α-1,3-galactosyltransferase. The hexose-α-1,3-galactosyltransferase may comprise or consist of a GlcNAc-α-1,3-galactosyltransferase, optionally the enzyme WbbP, or an enzymatically active fragment or variant thereof. The enzyme WbbP may be identified using the UniProt KB—Q53982 (Q53982_SHIDY). In some embodiments, WbbP may comprise or consist of the amino acid sequence SEQ ID NO:42. Thus, in some embodiments, the disaccharide consists of a GlcNAc at its reducing end and a galactose at its non-reducing end, the two hexoses linked via a α-1,3 bond.

In some embodiments, the method comprises forming the hexose disaccharide using the enzyme WbbP, or an enzymatically active fragment or variant thereof, followed by transferring a rhamnose moiety to the hexose disaccharide using the enzyme WbbR, or an enzymatically active fragment or variant thereof.

The hexose disaccharide may be formed using a hexose-α-1,3-rhamnosyltransferase or an enzymatically active fragment or variant thereof. For example, the hexose disaccharide may be formed using a galactose-α-1,3-rhamnosyltransferase, for example WsaD or an enzymatically active fragment or variant thereof. It will be appreciated in such embodiments that the hexose disaccharide is formed of a galactose at the reducing end and a rhamnose moiety at the non-reducing end. When the hexose disaccharide is formed using a galactose-α-1,3-rhamnosyltransferase, the enzyme WsaP optionally may also be used in the formation of the disaccharide, for example to attach a lipid to the galactose. The enzyme WsaP is derived from Geobacillus stearothermophilus. WsaP may be identified using the UniprotKB—Q7BG44 (Q7BG44_GEOSE). In some embodiments, the WsaP enzyme comprises or consists of SEQ ID NO:43.

Enzymatically active fragments or variants of WsaP may be derived from other Bacilli strains, for example Brevibacillus species and Paenibacillus species. The enzymatically active fragments or variants of WsaP may have at least 20%, 30%, at least 40%, at least 50%, at least 60%, at least 70%, at least 80%, at least 85%, at least 90%, at least 95%, at least 97%, at least 98% or at least 99% amino acid identity to WsaP.

The hexose disaccharide may be extended using a hexose-α-1,2-hexosyltransferase or an enzymatically active fragment or variant thereof to form a trisaccharide or tetrasaccharide prior to further extension from the rhamnose moiety at the non-reducing end of the trisaccharide or tetrasaccharide using a heterologous bacterial enzyme GacC and/or GacG or an enzymatically active homologue, variant or fragment thereof. Exemplary hexose-α-1,2-hexosyltransferases may include, but not be limited to WsaC and WsaE. WsaC may be identified by the UniProtKB—Q7BG54 (Q7BG54_GEOSE). Optionally, WsaC comprises or consists of SEQ ID NO: 44. WsaE may be identified by the UniProtKB—Q7BG51 (Q7BG51_GEOSE). Optionally, WsaE may comprise or consist of SEQ ID NO:45.

When the method further comprises forming the hexose trisaccharide, two monosaccharides may be linked together as described for the disaccharide, followed by the transfer of a further hexose to the non-reducing end of the disaccharide using an additional hexosyltransferase. The additional hexosyltransferase may comprise hexose-rhamnosyltransferases, such that a rhamnose moiety is transferred to the non-reducing end. Suitable hexose-rhamnosyltransferases may include any of the hexose-rhamnosyltransferases described herein. Suitable hexose-rhamnosyltransferases may include a rhamnose-α-1,3-rhamnosyltransferase, for example the enzyme WbbQ or WsaC, or an enzymatically active variant or fragment thereof. WbbQ may be identified using the UniProtKB—AOA090NIC3 (AOA090NIC3_SHIDY). In some embodiments, WbbQ comprises or consists of SEQ ID NO:46.

In some embodiments, the hexose trisaccharide is formed using a rhamnose-α-1,3-rhamnosyltransferase which is not GacC.

Further information regarding exemplary hexosyltransferases for use in the present invention are provided in the Examples.

The hexose monosaccharide, disaccharide or trisaccharide to which the rhamnose moiety is transferred may be linked to a lipid. Thus, step i) may comprise transferring a rhamnose moiety to a lipid-linked hexose monosaccharide, disaccharide or trisaccharide. The link between the hexose monosaccharide, disaccharide or trisaccharide may comprise an undecaprenyl-diphosphate.

The method may further comprise a step (step (iii)) of conjugating the rhamnose polysaccharide to an acceptor molecule using an O-oligosaccharyltransferase capable of recognising the hexose monosaccharide at the reducing end of the rhamnose polysaccharide to form a rhamnose glycoconjugate.

O-oligosaccharyltransferases are enzymes used to catalyse the transfer of a carbohydrate moiety to a target protein, in a process known as protein glycosylation. Protein glycosylation is the process of covalently attaching carbohydrate moieties, i.e., a polysaccharide, to a protein substrate. O-oligosaccharyltransferases function by cleaving a phosphate-monosaccharide bond at a reducing end of a polysaccharide. To be capable of interacting with the substrate, the O-oligosaccharyltransferase must be capable of recognising the first two monosaccharides after the phosphate bond. The substrate may otherwise be referred to as an acceptor. Thus, the acceptor molecule may comprise a peptide or a protein. This results in the formation of a glyconjugate comprising the rhamnose polysaccharide of the invention. Such glyconjugates are particularly useful as antigens, which can be used in immunogenic compositions or vaccines. In addition, when the method is performed in a bacterium, the process of glycosylation leads to the presentation of the glycoconjugate on the surface of the bacterium. This enables the glycoconjugate to be isolated from the bacterium for further use, or alternatively enables the whole bacterium to be used as an antigen, which can be used in an immunogenic composition or vaccine.

In some embodiments, the O-oligosaccharyltransferase is capable of recognising a glucose or glucose derivative. In such embodiments, the hexose monosaccharide at the reducing end of the rhamnose polysaccharide will be a glucose or a glucose derivative, such as N-acetyl glucosamine (GlcNAc).

The O-oligosaccharyltransferase may comprise PglB, PglL, PglS or WsaB or a enzymatically active homologue, fragment or variant thereof.

The PglB enzyme may be derived from a Campylobacter species, for example Campylobacter jejuni or Campylobacter lari. Without wishing to be bound by theory, it is believed that the PglB enzyme is capable of recognising any hexose except for glucose.

The PglL enzyme may derived from Neisseria meningitides. It is believed that the PglL enzyme is capable of recognising any hexose except for glucose.

The PglS enzyme may be derived from Acinetobacter species. It is believed that the PglS enzyme is capable of recognising glucose.

The WsaB enzyme is derived from Geobacillus stearothermophilus. Enzymatically active variants of the WsaB enzyme can be derived from other Geobacillus species.

In some embodiments, the O-oligosaccharyltransferase is derived from a bacterial species heterologous to the bacteria in which the method is performed.

The method may further comprise an additional step of purifying the rhamnose glycoconjugate. Purifying may comprise high performance liquid chromatography (HPLC), for example recycling-HPLC, affinity or size exclusion chromatography. Other suitable methods of purification will be known to the skilled person.

It will be appreciated that the method can be carried out at an industrial scale. As the skilled person will be aware, the bacteria in which the method can be performed are grown in liquid media. Such liquid media comprising the bacteria can be used to fill an industrial scale bioreactor, for example at a volume of at least 50, 100 or 1000 litres. This advantageously results in the synthesis of a substantial amount of the polysaccharide product of the invention.

A commonly used liquid media is Luria Broth, which may otherwise be referred to as Lysogeny Broth. Other liquid media will be known to the skilled person.

When the method is performed in bacteria, the method may be a fed-batch method. “Fed batch” is a term familiar to a person skilled in the art. Nevertheless, for the purposes of clarity, “fed batch” will be understood to refer to a method of synthesis in which nutrients are supplied to the bacteria via the liquid media during cultivation.

Suitable nutrients will be known to the skilled person. Some exemplary, but non-limiting nutrients may include a rhamnose moiety, a hexose other than a rhamnose moiety and/or divalent cations including, but not limited to, magnesium and/or manganese.

In some embodiments, the rhamnose moiety comprises rhamnose. Rhamnose may be supplied to the liquid media in the D or the L isoform, preferably the L isoform.

Which hexose other than a rhamnose moiety is supplied to the liquid media depends on the composition of the rhamnose polysaccharide produced by the method. If the hexose monosaccharide, disaccharide or trisaccharide to which the rhamnose moiety is transferred comprises glucose, then the skilled person will appreciate that a suitable nutrient to be supplied to the liquid media would be glucose. If the hexose monosaccharide, disaccharide or trisaccharide comprises galactose, then the skilled person will appreciate that a suitable nutrient to be supplied to the liquid media would be galactose. Thus, the hexose for supply to the liquid media may be selected from one or more of allose, altrose, glucose, mannose, xylose, idose, galactose, talose, diacetylbacillosamine, GalNAc or GlcNAc, as appropriate.

The rhamnose moiety and/or other hexose may (each) be supplied to the liquid media at a final concentration in the liquid media of 0.1, 0.25, 0.5, 1, 2, 3, 4, 5, 6, 7, 8, 9, 10 or 15 g/L. In some embodiments, the rhamnose moiety and/or other hexose is (each) supplied to the liquid media at a final concentration in the liquid media of about 4 g/L.

The rhamnose moiety and/or other hexose may (each) be supplied to the liquid media at a final concentration in the liquid media of 0.05, 0.1, 0.15, 0.2, 0.25, 0.3, 0.4, 0.5, 0.6, 0.7, 0.8, 0.9 or 1.0 mg/ml.

In embodiments, the rhamnose moiety is supplied to the liquid media as L-rhamnose. L-rhamnose may be supplied to the liquid media at a final concentration in the liquid media of 0.05, 0.1, 0.15, 0.2, 0.25, 0.3, 0.4, 0.5, 0.6, 0.7, 0.8, 0.9 or 1.0 mg/mL When magnesium is fed to the liquid media, this may be supplied in the form of MgSO4 or MgCl₂. The MgSO4 or MgCl₂ may be supplied to the liquid media to form a final concentration in the media of between 0 and 10 mM.

Prior to step i), when the method is performed in a bacterium the method may further comprise the introduction of one or more nucleic acids encoding one or more of the enzymes described herein into the bacterium. For example, the method may further comprise the introduction of a nucleic acid encoding the O-oligosaccharyltransferase and/or a nucleic acid encoding the hexose-β-1,4-rhamnosyltransferase, the hexose-α 1,2-rhamnosyltransferase, the hexose-α-1,3-rhamnosyltransferase, or an enzymatically active fragment or variant thereof into the bacterium. In some embodiments, the method further comprises the introduction of a nucleic acid encoding the bacterial enzyme GacC and/or the bacterial enzyme GacG or one or more enzymatically active homologue(s), variant(s) or fragment(s) thereof into the bacterium. The enzyme can then be expressed from its respective nucleic acid. The nucleic acid(s) encoding the one or more enzymes may further comprise a nucleic acid sequence encoding an endogenous or constitutive promoter and/or an artificial ribosome binding site.

Methods for the introduction of one or more nucleic acids into a bacterium are well known to those skilled in the art. One commonly used method is that of transformation. As used herein, transforming or transformation (which may otherwise be referred to as transfecting or transfection) refers to the process of introducing free nucleic acid into a cell by allowing the nucleic acid to cross the plasma membrane of the cell. By free nucleic acid, this will be understood to refer to nucleic acid which is not contained within a virus, virus-like particle or other organism; i.e., the nucleic acid is independent of an organism (although it will be appreciated that the nucleic acid may be derived or isolated from the nucleic acid sequence of an organism).

Methods of transfection typically involve altering the plasma membrane such that free nucleic acid can cross the plasma membrane (for example, electroporation methods) or complexing the free nucleic acid with a reagent that enables the free nucleic acid to cross the plasma membrane.

It will be appreciated that the nucleic acid for transfection may be in the form of a plasmid, this being a circular strand of nucleic acid. Hence, a plasmid may comprise one or more nucleic acid(s) encoding the one or more enzymes.

The nucleic acid is typically DNA, although RNA may also or alternatively be envisaged.

Transfecting may comprise polyethylenimine, poly-L-lysine, calcium phosphate, electroporation or liposomal-based methods. In embodiments, transfecting may comprise polyethylenimine, calcium phosphate or liposomal-based methods.

It will be appreciated that a variety of liposomal-based reagents are available commercially for liposomal-based methods of transfection. Liposomal methods may include, but may not be limited to, lipofectamine-based transfection or FuGENE®HD (Promega Corporation, Wisconsin, USA)-based transfection.

Further information regarding transformation/transfection techniques may be found in Current Protocols in Molecular Biology (2019) which is incorporated herein by reference.

The plasmid may further comprise appropriate regulatory sequences, including promoter sequences, terminator fragments, enhancer sequences, marker genes and/or other sequences. For further details see, for example, Sambrook & Russell, Molecular Cloning: A Laboratory Manual: 3^{rd} edition.

The plasmid may be further engineered to contain regulatory sequences that act as enhancer and promoter regions and lead to efficient transcription of the fusion protein sequence carried on the construct. Many parts of the regulatory unit are located upstream of the coding sequence of the heterologous gene and are operably linked thereto. The regulatory sequences can direct constitutive or inducible expression of the heterologous coding sequence. Such regulatory sequences are especially suitable if expression is wanted to occur in a time specific manner. Expression may be induced by supplying the liquid media with an inducer. The inducer may comprise or consist of arabinose, IPTG or rhamnose. Regulatory sequences which can direct inducible expression when exposed to arabinose, IPTG or rhamnose will be known to the skilled person.

Arabinose may be supplied to the liquid media at a final concentration in the liquid media of 1, 2, 3, 4, 5, 6, 7, 8, 9 or 10 g/L. Optionally, arabinose is supplied to the liquid media at a concentration of about 2 g/L.

IPTG may be supplied to the liquid media at a final concentration in the liquid media of 0.1 to 5 mM. In some embodiments, IPTG is supplied to the liquid media at a final concentration in the liquid media of 0.1 to 2 mM, preferably at a concentration of about 1 mM.

L-rhamnose may be supplied to the liquid media at a final concentration of 0.05, 0.1, 0.15, 0.2, 0.25, 0.3, 0.4, 0.5, 0.6, 0.7, 0.8, 0.9, 1.0 mg/mL as an inducer.

Also provided is a product obtainable using the method according to the first aspect. A product obtainable by the method according to the first aspect is especially pure and homogenous due to its synthetic method of production. The product of this invention is therefore ideally suited to commercial use, for example for the production on a large scale for use as an antigen or for use in research applications.

According to a third aspect there is provided a synthetic streptoccocal polysaccharide, the polysaccharide having a non-reducing end comprising a linear chain of rhamnose moieties and a reducing end comprising a hexose monosaccharide, disaccharide or trisaccharide, the hexose monosaccharide, disaccharide or trisaccharide being as described in relation to the method aspect. The polysaccharide comprises an α-1,3 bond or a an α-1,2 bond between the hexose monosaccharide, disaccharide or trisaccharide and the linear chain of rhamnose moieties, or the polysaccharide comprises an β-1,4 bond between the hexose monosaccharide, disaccharide or trisaccharide and the linear chain of rhamnose moieties and the hexose monosaccharide, disaccharide or trisaccharide does not comprise N-acetylglucosamine.

As the inventors have found, the naturally occurring GAC from S. pyogenes comprises a GlcNAc (N-acetylglucosamine) monosaccharide linked by a β-1,4 glycosidic bond to a linear chain of rhamnose monosaccharides. By altering this natural composition of the reducing end sugars, the inventors have generated a synthetic polysaccharide which retains the chemical composition and antigenic capacity of the alpha-1,2-alpha-1,3 rhamnose disaccharide repeat units of GAC, while enabling production of the polysaccharide at an industrial scale and at high levels of purity and tightly regulated size distribution to increase product length homogeneity.

Thus, typically, the polysaccharide comprises a polysaccharide or a fragment or variant thereof selected from the group consisting of a Group A, Group B, Group C and Group G carbohydrate.

In some embodiments, the polysaccharide comprises an α-1,3 bond between the hexose monosaccharide, disaccharide or trisaccharide and the linear chain of rhamnose moieties. The hexose monosaccharide disaccharide or trisaccharide may comprise N-acetylglucosamine, N,N′-diacetylbacillosamine, glucose or galactose.

In some embodiments, the polysaccharide comprises an α-1,2 bond between the hexose monosaccharide, disaccharide or trisaccharide and the linear chain of rhamnose moieties. The hexose may comprise galactose.

In some embodiments, the polysaccharide comprises a β-1,4 bond between the hexose monosaccharide, disaccharide or trisaccharide and the linear chain of rhamnose moieties and the hexose comprises glucose.

According to a fourth aspect, there is provided a streptococcal rhamnose glycoconjugate comprising the streptococcal polysaccharide according to the third aspect conjugated to an acceptor. Glyconjugates have strong antigenic potential and so rhamnose glyconjugates of the invention have particular utility in raising an immune response for example as part of or as an immunogenic composition or vaccine.

In embodiments, the polysaccharide is conjugated to the acceptor at the reducing end of the polysaccharide. The acceptor may comprise a peptide or a protein.

In some embodiments, the streptococcal rhamnose glycoconjugate is expressed on the surface of a bacterial host cell, optionally a gram negative bacterium such as E. coli. Thus, the invention also encompasses a bacterial host cell comprising the streptococcal rhamnose glycoconjugate of the fourth aspect on its cell surface. Conveniently, expression on the cell surface of the bacterial host cell enables ease of isolation of the glycoconjugate. Even more conveniently, this means that the bacterial host cell which comprises the streptococcal rhamnose glycoconjugate on its cell surface can be used as a component of or an immunogenic composition or vaccine without requiring isolation of the glyconjugate from the bacterial host cell. This reduces the time and cost necessary to produce the glyconjugate for downstream use as an immunogenic composition or vaccine.

Thus, according to a fifth aspect there is provided a bacterial host cell comprising a hexose-β-1,4-rhamnosyltransferase, a hexose-α-1,2-rhamnosyltransferase or a hexose-α-1,3-rhamnosyltransferase, or an enzymatically active fragment or variant thereof and the heterologous bacterial enzyme GacC and/or GacG or an enzymatically active homologue, variant or fragment thereof as described herein.

The bacterial host cell may be heterologous to the species from which the hexose-β-1,4-rhamnosyltransferase, a hexose-α-1,2-rhamnosyltransferase or a hexose-α-1,3-rhamnosyltransferase, or an enzymatically active fragment or variant thereof is derived.

Optionally, the bacterial host cell is a gram-negative bacterium such as E. coli. The bacterial host cell may comprise the enzymes described herein and/or the nucleic acid sequences encoding the enzymes.

According to a sixth aspect, there is provided an immunogenic composition or vaccine comprising the rhamnose polysaccharide of the second or third aspect or the streptococcal glycoconjugate according to the fourth aspect. The immunogenic composition or vaccine may further comprise a pharmaceutically acceptable and/or sterile excipient, carrier and/or diluent.

In some embodiments, the immunogenic composition or vaccine further comprises an antigen, polypeptide and/or adjuvant.

The composition may further comprise a pharmaceutically acceptable carrier, diluent or excipient. A “pharmaceutically acceptable carrier” as referred to herein is any physiological vehicle known to those of ordinary skill in the art useful in formulating pharmaceutical compositions. A “diluent” as referred to herein is any substance known to those of ordinary skill in the art useful in diluting agents for use in pharmaceutical compositions. The agent may be mixed with, or dissolved, suspended or dispersed in the carrier, diluent or excipient.

The composition may be in the form of a capsule, tablet, liquid, ointment, cream, gel, hydrogel, aerosol, spray, micelle, transdermal patch, liposome or any other suitable form that may be administered to an animal suffering from, or at risk of developing a disease, condition or infection with a streptococcal aetiology.

The compositions and/or vaccines of this invention may be formulated for oral, topical (including dermal and sublingual), intramammary, parenteral (including subcutaneous, intradermal, intramuscular and intravenous), transdermal and/or mucosal administration. In embodiments the compositions and vaccines of this invention may be formulated for parenteral administration, optionally subcutaneous, intradermal, intramuscular and/or intravenous administration.

There is also provided the rhamnose polysaccharide of the second or third aspect, the streptococcal glycoconjugate according to the fourth aspect, or the immunogenic composition or vaccine according to the sixth aspect for use in raising an immune response in an animal or for use in treating or preventing a disease, condition or infection with a streptococcal aetiology.

The animal may be any mammalian subject, for example a dog, cat, rat, mouse, human, sheep, goat, donkey, horse, cow, pig and/or chicken.

In embodiments, the animal is an ovine animal, a caprine animal, an equine animal, a porcine animal, a bovine animal or a human. In embodiments, the animal is an ovine animal. By “ovine animal”, this will be understood to include sheep.

The skilled person will appreciate that the term “caprine” includes goats, while “bovine” includes cattle. Equine is a term that will be understood to include horses. As used herein, the term “porcine” includes pigs.

An immune response which contributes to an animal's ability to resolve an infection/infestation and/or which helps reduce the symptoms associated with an infection/infestation may be a referred to as a “protective response”. In the context of this invention, the immune responses raised through exploitation of the rhamnose polysaccharides described herein may be referred to as “protective” immune responses. The term “protective” immune response may embrace any immune response which: (i) facilitates or effects a reduction in host pathogen burden; (ii) reduces one or more of the effects or symptoms of an infection/infestation; and/or (iii) prevents, reduces or limits the occurrence of further (subsequent/secondary) infections.

Thus, a protective immune response may prevent an animal from becoming infected/infested with a particular pathogen and/or from developing a particular disease or condition.

An “immune response” may be regarded as any response which elicits antibody (for example IgA, IgM and/or IgG or any other relevant isotype) responses and/or cytokine or cell mediated immune responses. The immune response may be targeted to the rhamnose polysaccharide of the invention. For example, the immune response may comprise antibodies which have affinity for epitopes of or the entire rhamnose polysaccharide.

Also provided is a method of treating an animal having a disease, condition or infection with a streptococcal aetiology, the method comprising administering the animal a therapeutically effective amount of the rhamnose polysaccharide of the second or third aspect, the streptococcal glycoconjugate according to the fourth aspect, or the immunogenic composition or vaccine according to the sixth aspect.

A therapeutically effective amount will be understood to refer to an amount sufficient to eliminate, reduce or prevent a disease, condition or infection with a streptococcal aetiology.

The rhamnose polysaccharide, glyconjugate or the immunogenic composition or vaccine may be administered as a single dose or as multiple doses. Multiple doses may be administered in a single day (e.g., 2, 3 or 4 doses at intervals of e.g., 3, 6 or 8 hours). The agent may be administered on a regular basis (e.g., daily, every other day, or weekly) over a period of days, weeks or months, as appropriate.

It will be appreciated that optimal doses to be administered can be determined by those skilled in the art and will vary depending on the particular agent in use, the strength of the preparation, the mode of administration and the advancement or severity of the disease, condition or infection with a streptococcal aetiology. Additional factors depending on the particular subject being treated will result in a need to adjust dosages, including subject age, weight, gender, diet, and time of administration. Known procedures, such as those conventionally employed by the pharmaceutical industry (e.g., in vivo experimentation, clinical trials, etc.), may be used to establish specific formulations for use according to the invention and precise therapeutic dosage regimes.

Also provided is a kit of parts, the kit comprising:
(i) A nucleic acid sequence encoding a hexose-β 1,4-rhamnosyltransferase, a hexose-α-1,2-rhamnosyltransferase or a hexose-α 1,3-rhamnosyltransferase, or an enzymatically active fragment or variant thereof; and
(ii) A nucleic acid sequence encoding the heterologous bacterial enzyme GacC and/or GacG or an enzymatically active homologue, variant or fragment thereof.

Suitable nucleic acid sequences for the kit of parts are as described herein in relation to the method of the invention.

In some embodiments, the kit further comprises one or more nucleic acid sequences encoding an O-oligosaccharyltransferase as described herein.

Further nucleic acid sequences which the kit may comprise may include one or more nucleic acid sequences encoding one or more of the following 12 enzymes GacA, GacD, GacE, GacF, GacH, Gacl, GacJ, GacK and GacL, or one or more enzymatically active homologue(s), variant(s) or fragment(s) thereof.

In some embodiments, the kit further comprises a nucleic acid sequence encoding GacA, or an enzymatically active homologue, variant or fragment thereof. In some embodiments, the kit comprises a nucleic acid sequence encoding GacG, or an enzymatically active homologue, variant or fragment thereof.

In some embodiments, the kit comprises nucleic acid sequences encoding GacG and GacC, or one or more enzymatically active homologue(s), variant(s) or fragment(s) thereof.

In some embodiments, the kit further comprises nucleic acid sequences encoding the enzymes GacA, GacD, GacE, and GacF or one or more enzymatically active homologues, fragments or variants thereof.

The kit may further comprise one or more nucleic acid sequences encoding a reporter gene.

The reporter sequence may encode a gene or peptide/protein, the expression of which can be detected by some means. Suitable reporter sequences may encode genes and/or proteins, the expression of which can be detected by, for example, optical, immunological or molecular means. Exemplary reporter sequences may encode, for example, fluorescent and/or luminescent proteins. Examples may include sequences encoding firefly luciferase (Luc: including codon-optimised forms), green fluorescent protein (GFP), red fluorescent protein (dsRed). One or both of the nucleic acid sequences described in (i) and (ii) of the kit may comprise the reporter sequence.

The kit may optionally further comprise bacteria, for example gram-negative bacteria such as E. coli. The bacteria may be heterologous to the bacterial species from which the hexose-β-1,4-rhamnosyltransferase, the hexose-α-1,2-rhamnosyltransferase, the hexose-α-1,3-rhamnosyltransferase or enzymatically active fragment or variant thereof is derived.

It will be appreciated that the plurality of nucleic acid sequences may be provided in one or a plurality of plasmids.

All of the features described herein (including any accompanying claims, abstract and drawings) may be combined with any of the above aspects in any combination, unless otherwise indicated.

### DETAILED DESCRIPTION

The invention will now be described by way of example with reference to the following figures, which show:

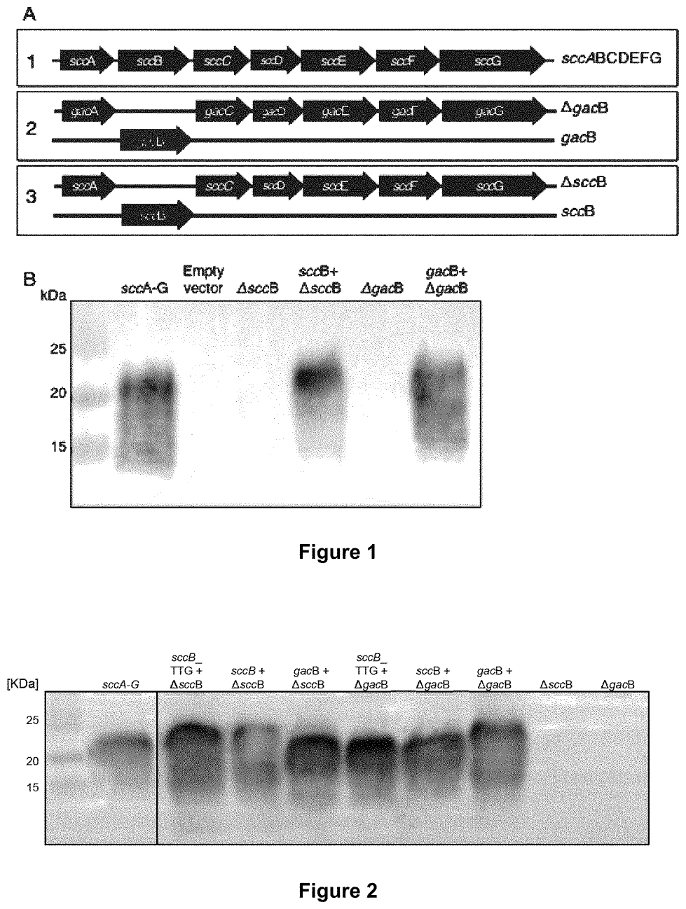

FIG. 1 A) shows a gene complementation strategy and map of S. pyogenes and S. mutans genes required to produce the rhamnose chain. S. mutans cluster: sccA (Smu0824), sccB (Smu0825), sccC (Smu0826), sccD (Smu0827), sccE (Smu0828), sccF (Smu0829), sccG (Smu0830). S. pyogenes cluster: gacA (M5005_Spy_0602), gacB (M5005_Spy_0603), gacC (M5005_Spy_0604), gacD (M5005_Spy_0605), gacE (M5005_Spy_0606), gacF (M5005_Spy_0607), gacG (M5005_Spy_0608). B) Bacterial complementation assay. Western blot of whole cells samples probed with anti-Group A antibody. Legends on the figure;

FIG. 2 shows a western blot of whole cell samples probed against anti-GAC antibody showing the complementation of ΔsccB or ΔgacB with sccB_TTG, sccB_ATG and gacB;

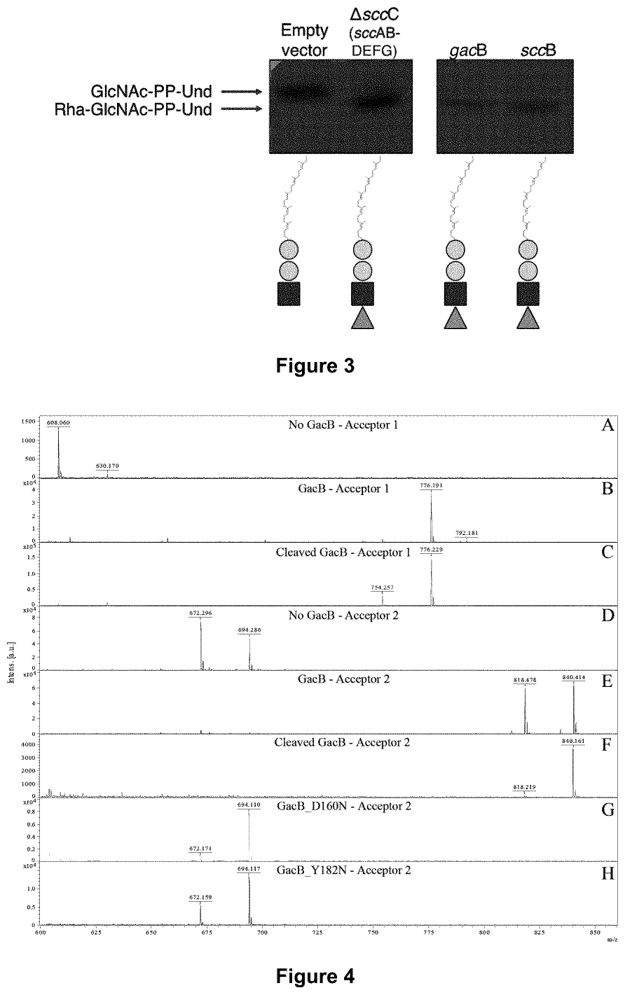

FIG. 3 shows a thin layer chromatography analysis of radiolabelled lipid-linked oligosaccharides extracted from E. coli cells expressing the empty vector, S. mutans SccAB-DEFG, S. pyogenes GacB or S. mutans SccB;

FIG. 4 shows an in vitro assessment of GacB's activity detected MALDI-MS. Spectra obtained from the products of the enzymatic reaction between dTDP-Rha and: A. Acceptor 1 (C13-PP-GlcNAc) B. Acceptor 1+GacB-GFP C. Acceptor 1+GacB cleaved (no GFP) D. Acceptor 2 (Phenol-O—C11-PP-GlcNAc). E. Acceptor 2+GacB-GFP. F. Acceptor 2+GacB cleaved (no GFP) G. Acceptor 2+GacB-D160N-F GFP H. Acceptor 2+GacB-Y182N-F-GFP;

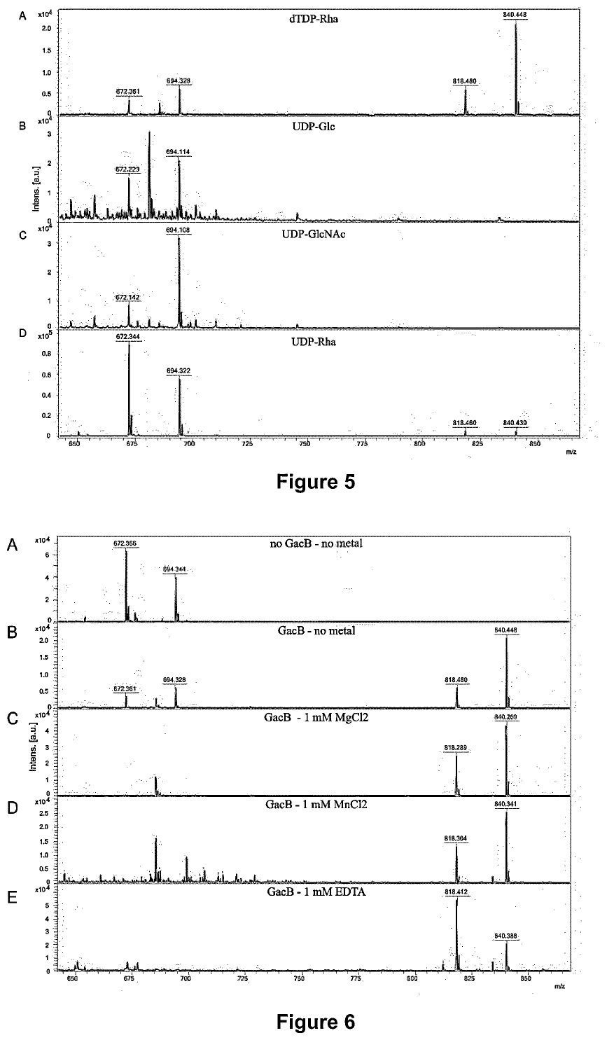

FIG. 5 shows an in vitro assessment of GacB's specificity towards different activated nucleotide sugar donors using MALDI-MS. Spectra obtained from the products of the enzymatic reaction between GacB-GFP, acceptor 2 and either dTDP-Rha (A), UDP-Glc (B), UDP-GlcNAc (C) or UDP-Rha (D). The conversion to the product (818 m/z and 840 m/z) was observed only when dTDP-Rha was used as nucleotide sugar donor;

FIG. 6 shows an in vitro assessment of GacB's metal ion dependency via MALDI MS. Spectra obtained from the products of the enzymatic reaction between dTDP-Rha, acceptor 2 (A), and either: GacB-GFP (B), 1 mM MgCl₂ (C), 1 mM MnCl₂ (D), or EDTA (E). The conversion to the product (818 m/z and 840 m/z) was observed in all conditions where GacB-GFP was present, regardless of the addition of metal ions or the metal chelator;

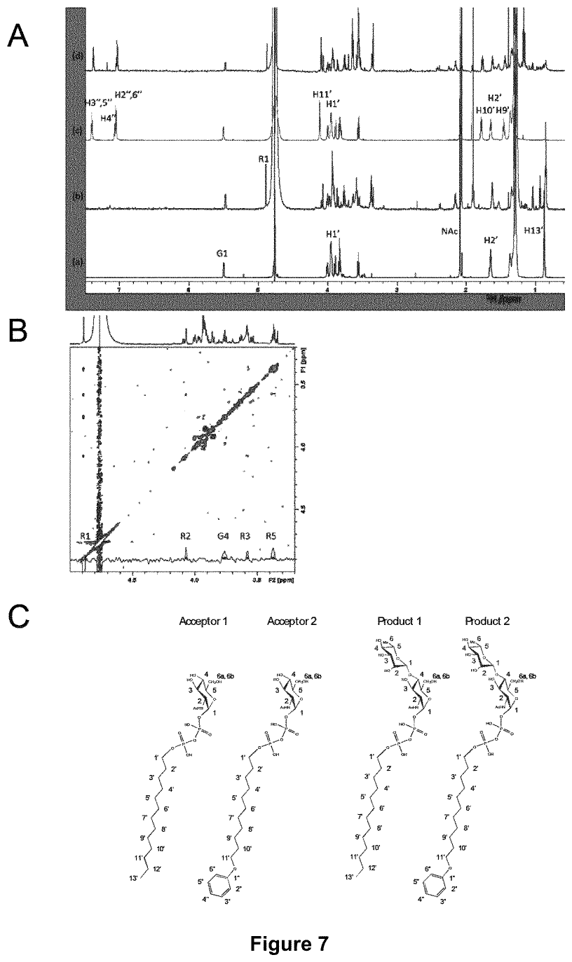

FIG. 7 shows A) 800 MHz ¹H NMR spectra of (a) acceptor substrate 1, (b) product 1, (c) acceptor substrate 2, (d) product 2. B) Partial 2D ROESY spectrum of the product 1 showing the correlations between the H1 of a β-L-Rha and protons of rhamnose (R) and GlcNAc (G). The F2 cross section through H1 of Rha is shown in red. C) The chemical structures with proton numbering.

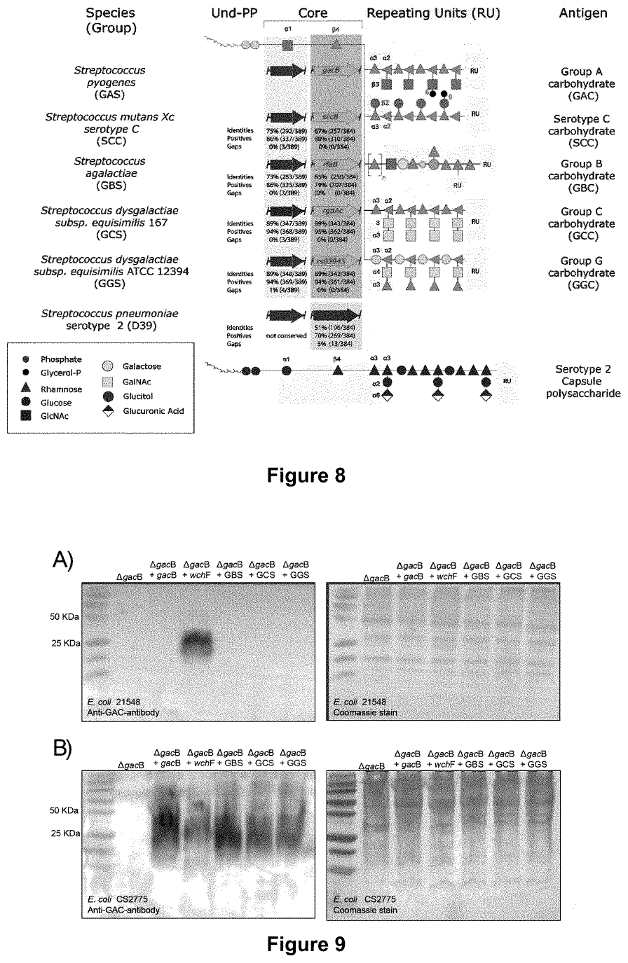

FIG. 8 shows a schematic representation of the RhaPS initiation within different Streptococcus species in comparison to the capsule polysaccharide in S. pneumoniae. RhaPS biosynthesis is initiate on Und-P by GacO (green background), followed by the action of GacB (turquoise), generating the conserved core structure Und-PP-GlcNac-Rha. Percentage of the amino acid sequence identity, positive amino acids, and gaps within the sequence compared to GacO or GacB are given below each homolog: S. mutans serotype c SccB, Streptococcus agalactiae (GBS) RfaB, Streptococcus dysgalactiae subsp. equisimilis 167 (GCS) RgpAc, Streptococcus dysgalactiae subsp. equisimilis ATCC 12394 (GGS) Rs03945. The specific carbohydrate composition extending the lipid linked core structure of each group are depicted on the right side. Repeating units (RU) of the carbohydrates are highlighted (light pink background), symbolic representation of the sugar residues is shown in the figure legend;

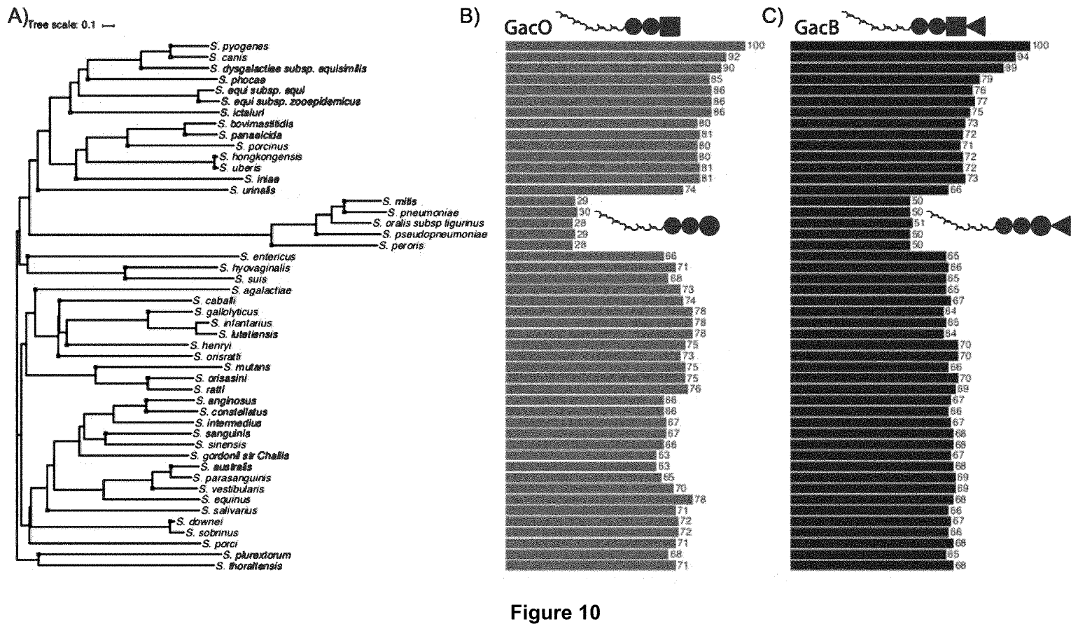

FIG. 9 shows (top) anti-lipid A and anti-GAC western blot of E. coli total cell lysate. WchF complementation of the dgacB gene cluster complements RhaPS biosynthesis in 21548 cells (lacking Und-PP-GlcNAc, inactive wecA gene), whilst no other GacB and homologous enzyme fail to initiate RhaPS biosynthesis. (Below) All gene combinations result in functional RhaPS biosynthesis in CS2775 cells (containing Und-PP-GlcNAc, functional wecA gene);

FIG. 10 A) shows phylogenetic relationships amongst forty-eight partially or completely sequenced streptococcal pathogens. The tree was constructed based a multiple sequence alignment of GacB homologs using the default neighbour-joining clustering method of Clustal Omega. The tree was plotted using iTOL online tool. Black squares at the branches indicate species with fully sequenced genomes. (B) Bar charts associates to each node indicate the percentage amino acid identity of the respective homologs to GacB (blue) or GacO (magenta);

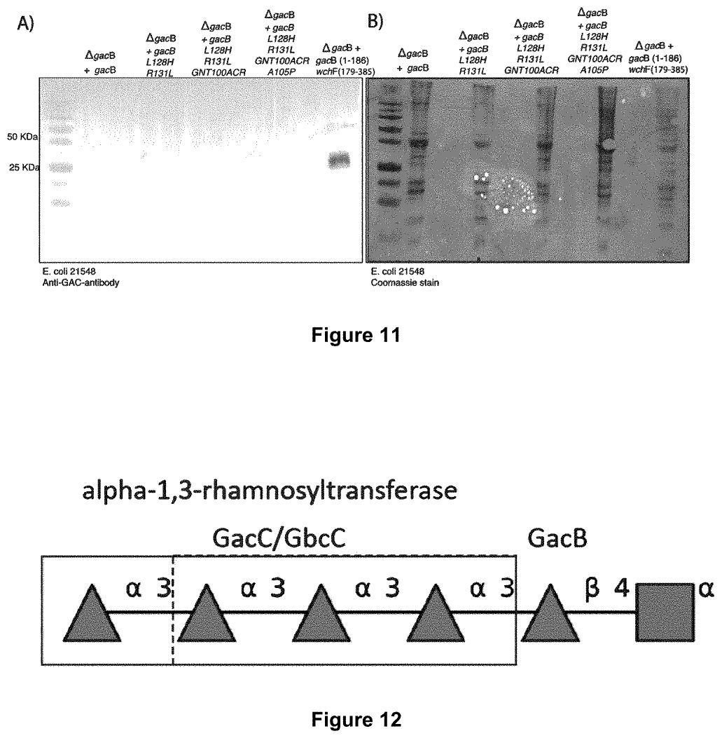

FIG. 11 Left) shows anti-GAC western blot of total cell lysate western blot of E. coli 21548 cells expressing dgacB gene cluster and either gacB, gacB-mutants or gacB-WchF chimera. The GacB-WchF chimera complements the dgacB RhaPScluster, suggesting that the N-terminal WchF domain is sufficient to alter the acceptor substrate specificity for GacB from Und-PP-GlcNAc to Und-PP-Glc.Right) Loading control—coomassie stained membrane after Western blotting;

FIG. 12 is a schematic diagram to show the composition of the naturally occurring GAC; and

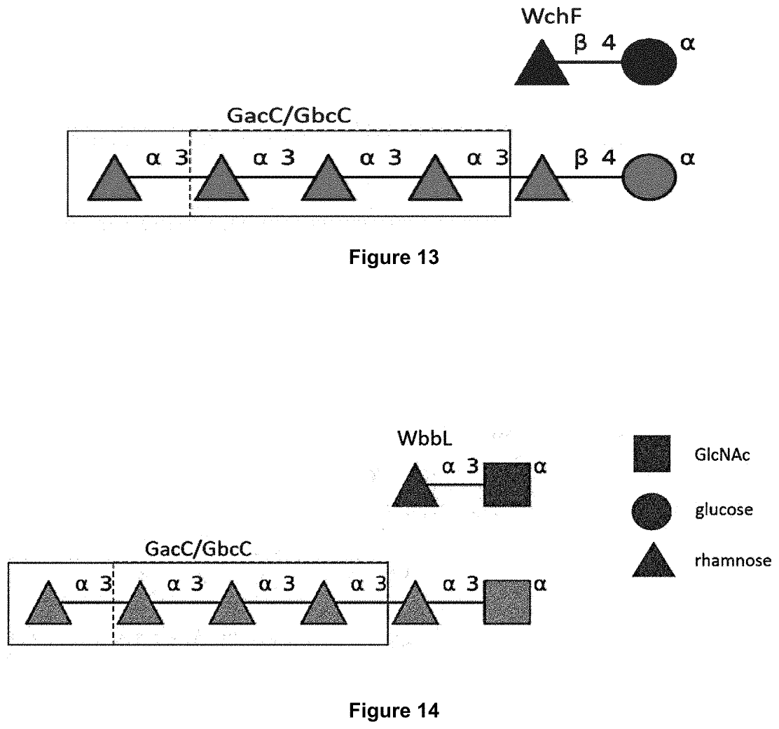

FIG. 13 is a schematic diagram to illustrate an embodiment of the invention;

FIG. 14 is a schematic diagram to illustrate another embodiment of the invention;

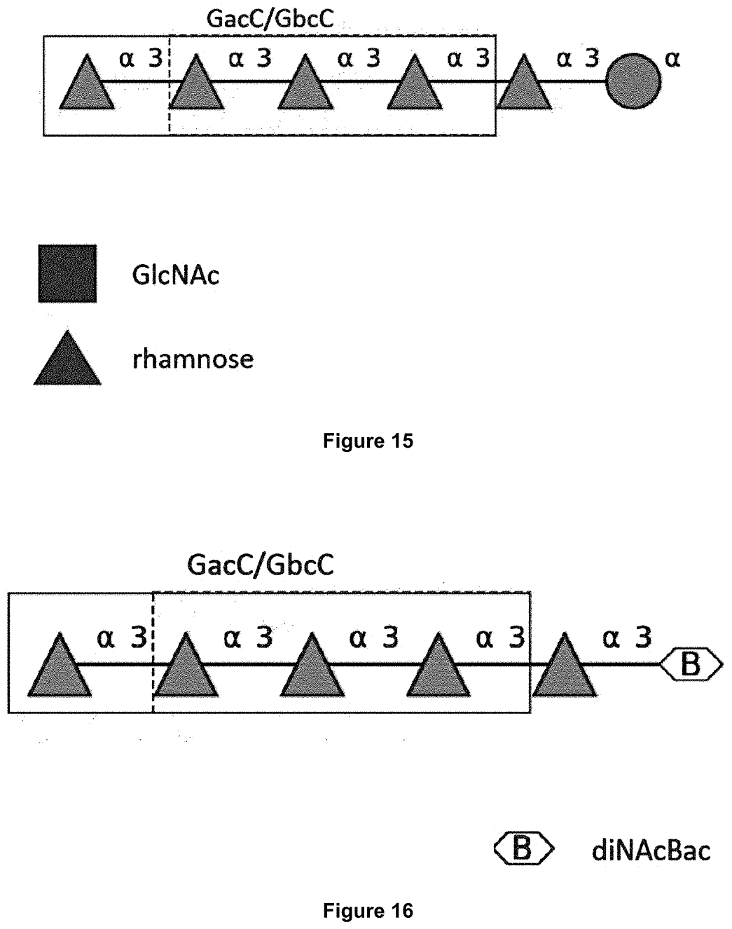

FIG. 15 is a schematic diagram to illustrate a further embodiment of the invention;

FIG. 16 is a schematic diagram to illustrate another embodiment of the invention;

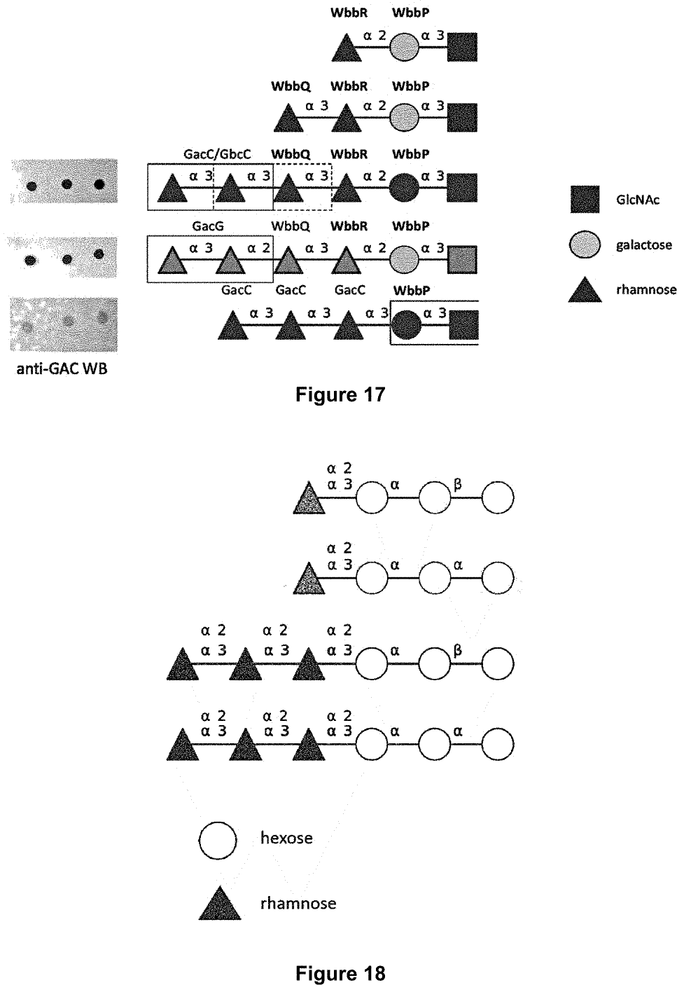

FIG. 17 is a schematic diagram to illustrate embodiments of the invention;

FIG. 18 is another schematic diagram to further illustrate the invention;

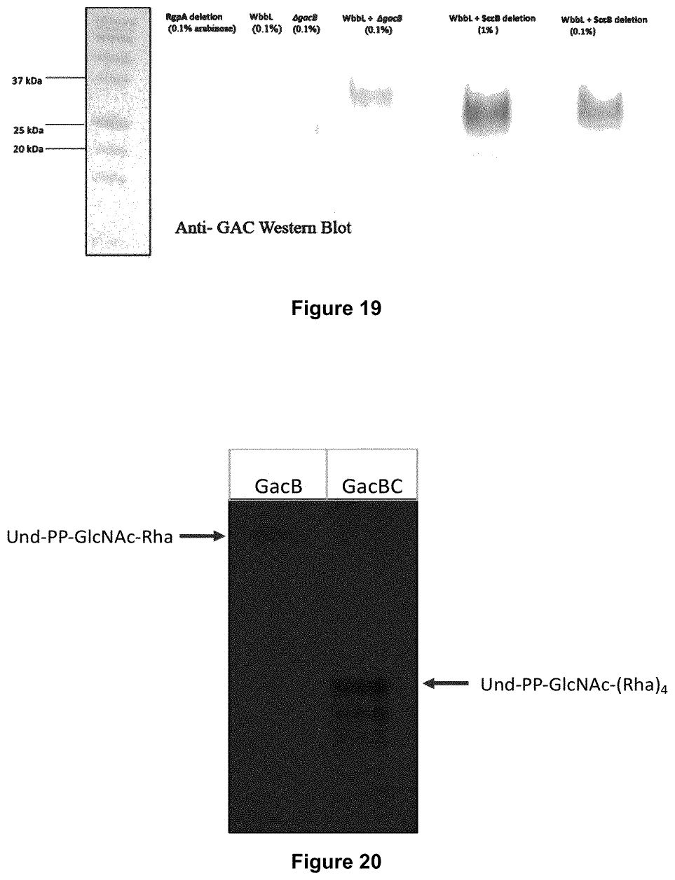

FIG. 19 is an anti GAC Western Blot to show that WbbL can be used instead of GacB or SccB in a method according to the invention. The figure shows an anti-GAC Western blot of total E. coli lysate from cells expressing the gene cluster RmlD-SccC-SccD-SccE-SccF-SccG (deltaSccB) and GacA-GacC-GacD-GacE-GacF-GacG (deltaGacB) complemented with empty plasmid controls or WbbL. Arabinose induction concentrations stated in %;

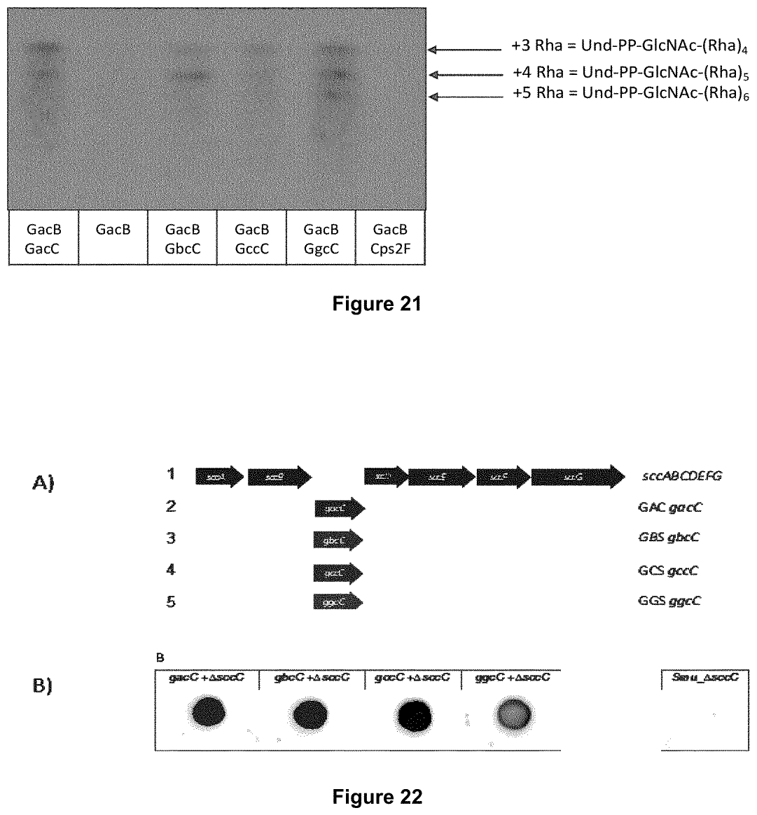

FIGS. 20 and 21 are images of radiolabelled lipid-linked oligosaccharides prepared in vivo;

FIG. 22 shows the results from E. coli complementation studies;

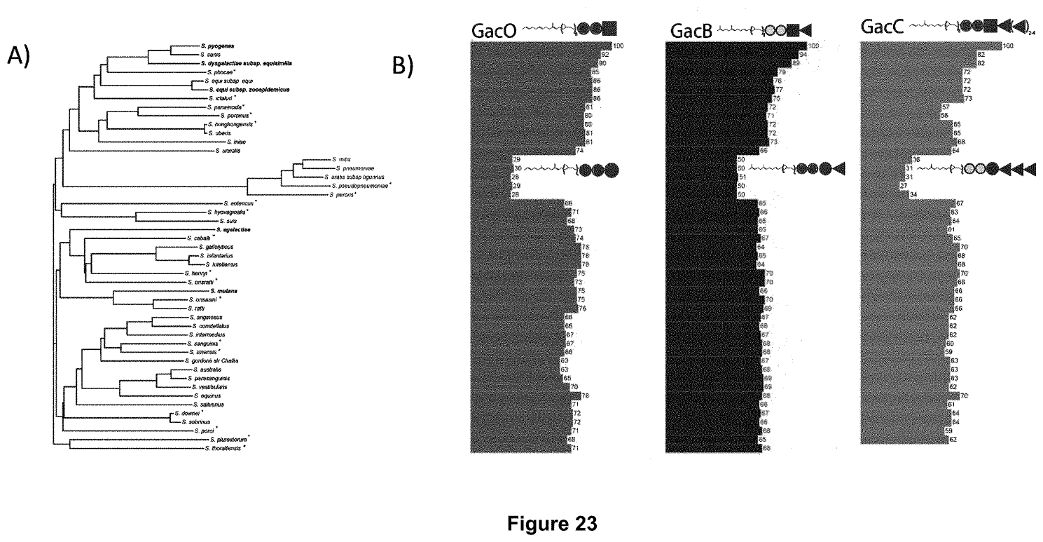

FIG. 23 shows the results of phylogenetic studies of the GacO, GacB and GacC enzymes from Streptococci spp.;

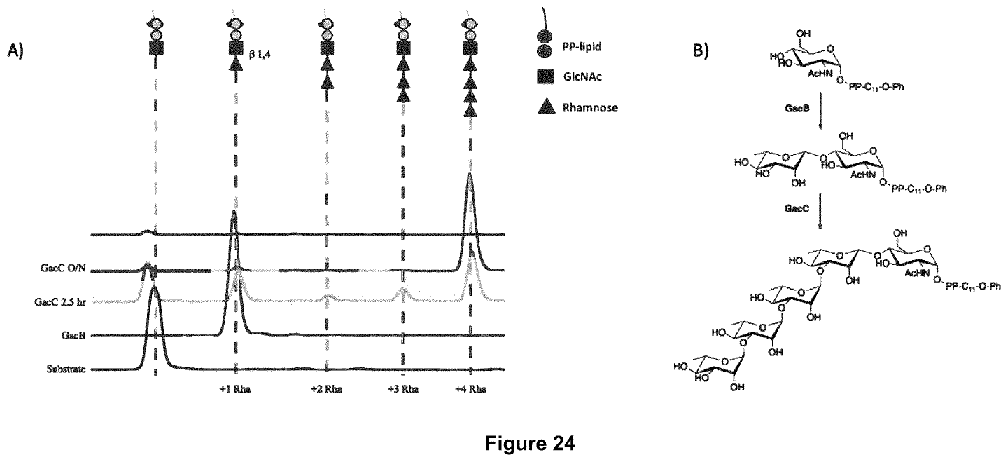

FIG. 24 shows the functional characterisation of GacC and how GacC installs poly-rhamnose to an adaptor/stem;

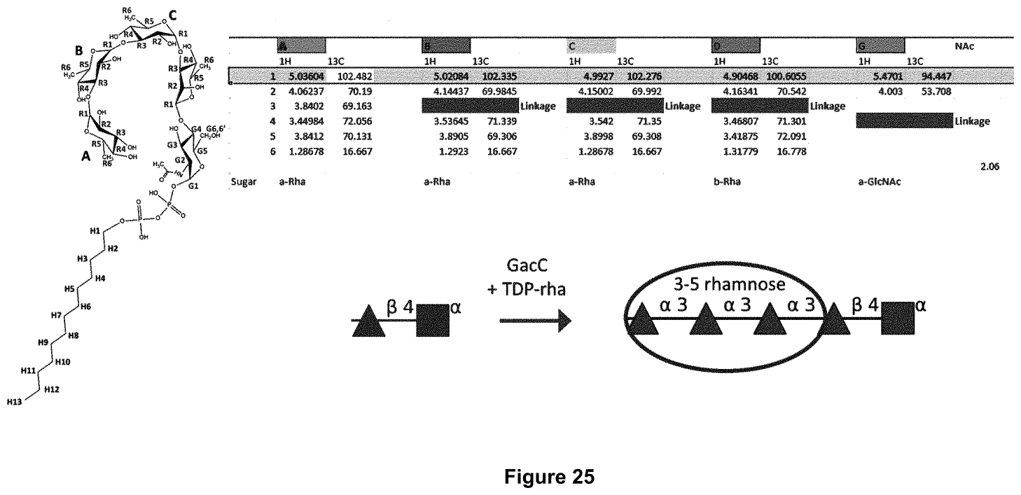

FIG. 25 shows assignment of proton and carbon sugar signals as obtained from 2D TOCSY and NOESY spectra and how this translates into the rhamnose polysaccharide molecule;

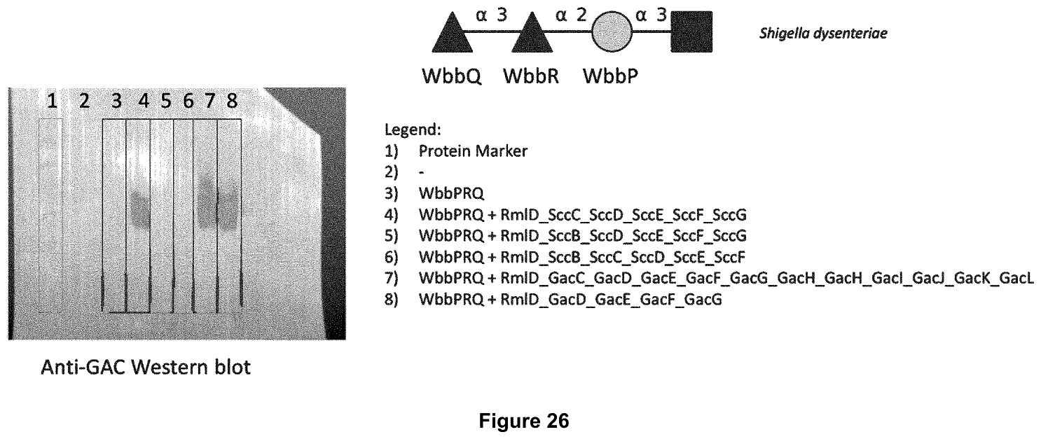

FIG. 26 shows a Western blot image obtained from generating rhamnose polysaccharides with a WbbPQR adaptor/stem;

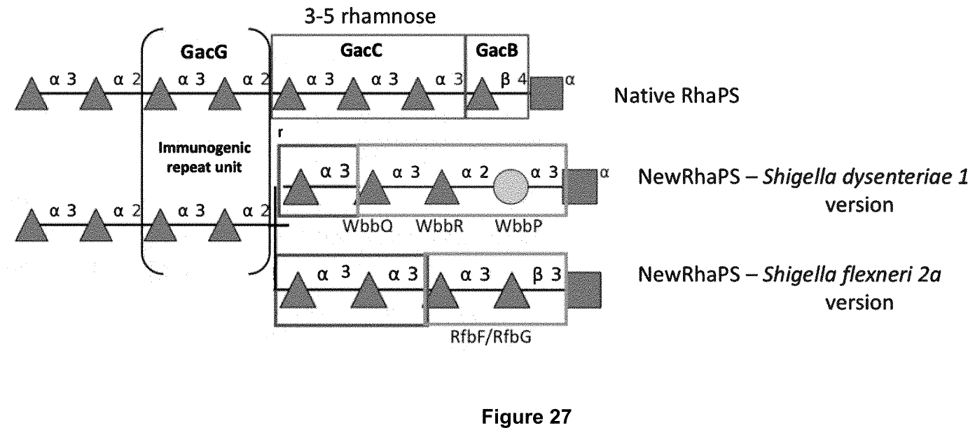

FIG. 27 shows a schematic of rhamnose polysaccharides generated from Shigella spp. adaptor/stem and GAC repeat units; and

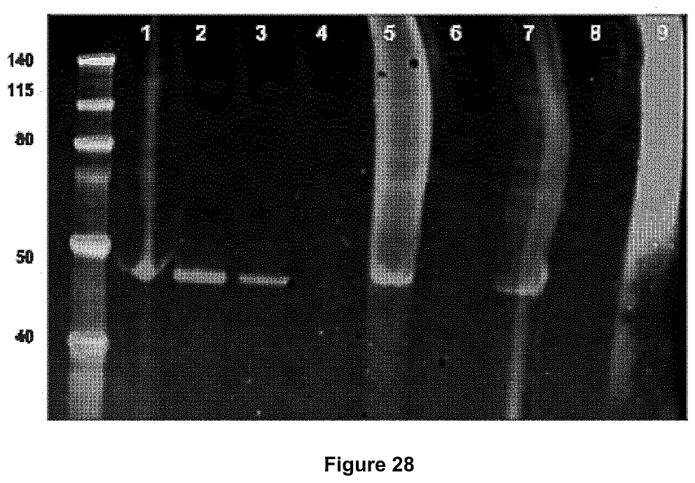

FIG. 28 shows rhmanose polysaccharides prepared in accordance with the present invention are capable of acting as substrates for an E. coli glycoconjugation system.

### EXAMPLE 1—GACB IS A α-D-GLCNAC β-1,4-L-RHAMNOSYLTRANSFERASE

### Introduction

S. pyogenes relies on different mechanisms to withstand the host's defences (1-5). These mechanisms are supported by the synthesis of a wide array of virulence factors, amongst which is the Group A Carbohydrate (GAC), a surface polysaccharide that constitutes between 40% and 60% of the bacterial cell wall (6-9). GAC is composed of a [→3)α-Rha(1→2)α-Rha(1→] rhamnose polysaccharide (RhaPS) backbone with a β-D-GlcNAc (1→3) side chain modifications on every α-1,2-linked rhamnose (9-11). Recent structural examinations and composition analysis of the GAC also suggest the presence of glycerol phosphate (GroP) (12), an observation that remained unnoticed for over fifty years (13,14). Further, Edgar et al. demonstrated that approximately 25% of GAC side chain GlcNAcs are decorated with GroP, imparting a negative charge to this polymer that has implications on S. pyogenes biology and defence mechanisms (12, 13, 15). This feature, previously identified in other surface glycans (16,17), provided new insight into the structural composition, biosynthesis and function of GAC.

GAC is proposed to be synthesised by twelve proteins, GacABCDEFGHIJKL, encoded in one gene cluster (i.e.: MGAS5005_spy0602-0613) that has been found in all S. pyogenes species identified so far (1, 18). Through sequencing of transposon mutant libraries, Le Breton et al. discovered that eight of these genes, gacABCDEFG and gacL are essential for S. pyogenes survival (4, 19). This information supports the observation by van Sorge et al., who identified via insertional mutagenesis that the first three genes of the cluster (gacABC) are essential (1).

It is currently hypothesized that the GAC is formed in five consecutive steps: (i) lipid-linked acceptor initiation, (ii) [-→3)α-Rha(1→2)α-Rha(1→] RhaPS backbone synthesis, (iii) membrane translocation, (iv) post-translocational chain modifications in the extracellular environment and (v) linkage to the peptidoglycan (9). The cytoplasmic pool of dTDP-rhamnose is supplied by the enzymes encoded in two separate gene clusters rm/ABC and gacA/rm/D (16).

Despite the recent findings, some pressing questions remain unanswered regarding the biosynthesis of the GAC. For example, the products of six of the twelve genes that constitute the GAC cluster (gacBCDEFG) have not yet been characterised, leaving the GAC initiation, RhaPS backbone biosynthesis and translocation steps unknown.

As a means of attaining more information on the GAC initiation step, we conducted an in-depth examination of the second enzyme encoded in the GAC gene cluster. Here we demonstrate that GacB, in disagreement with its preliminary genetic annotation and currently proposed action (8), is the first retaining rhamnosyltransferase that catalyses the transfer of L-rhamnose from dTDP-β6-L-rhamnose. GacB forms a β-1,4 glycosidic bond with the lipid-linked GlcNAc-diphosphate through a metal-independent mechanism. More importantly, our research on phylogenetically-related homologs from other important human pathogenic streptococci, in particular from the Lancefield groups B, C and G streptococci, reveal that the role of GacB is well conserved within the Streptococcus genus, suggesting a common first committed step for the production of RhaPS from all Lancefield groups.

Experimental Procedures

Bioinformatics Analysis

Alignment of protein sequences was performed using NCBI Blast Global align (https://goo.gl/vB9zmD) and ClustalOmega (https://goo.gl/8FbvYP) (49). Molecular weight predictions were obtained using the ProtParam tool at the Expasy server (http://www.expasy.org/). Topological predictions were generated using both SpOctopus (http://octopus.cbr.su.se/) and the TMHMM algorithms (www.cbs.dtu.dk/services/TMHMM/).

Secondary structure predictions were generated using either Phyre2 (https://goo.gl/zrGKJ7) or RaptorX (raptorx.uchicago.edu) homology recognition engines, and these structures were viewed and analysed using the PyMOL Molecular Graphics System (educational version 1.8 Schrödinger, LLC). The Carbohydrate Active Enzymes database (CAZy) (http://www.cazy.org/) (50) was examined to obtain information about the classification and characterization of carbohydrate active enzymes. Phylogeny relationships were established using Clustal Omega, Clustal X and the interactive tree of life iTOL (22).

Bacterial Strains and Growth Conditions

E. coli strains DH5a and MC1061 were used indistinctively as host strains for the propagation of recombinant plasmids and plasmid integration. E. coli CS2775, a strain lacking the Rha modification on the lipopolysaccharide, was used as the host strain to evaluate the production of RhaPS. E. coli 21548 is an Und-PP-GlcNAc deficient strain that contains a wecA deletion, serving as a negative control for the production of RhaPS. E. coli strain C43 (DE3) was used for the production of recombinant protein. All E. coli strains were grown in LB media. Unless otherwise indicated, all bacterial cultures were incubated at 37° C. in a shaking incubator at 200 rpm. Where necessary, media were supplemented with one or more antibiotics to the following final concentration: carbenicillin (Amp) at 100 μg/μL, erythromycin (Erm) at 300 μg/μL or kanamycin (Kan) at 50 μg/mL.

Molecular Genetic Techniques

Table 1 shows the DNA sequence of the forward and reverse oligonucleotide primer pairs used to amplify, delete, or mutagenise the genes of interest. All primers were obtained from Integrated DNA Technologies (IDT). All PCR reactions were performed using a SimpliAmp™ Thermal Cycler from ThermoFisher Scientific with standard procedures. Constructs were cloned using standard molecular biology procedures, including restriction enzyme digest and ligation. All constructs were validated with DNA sequencing.

| | | Gene | Amplified | | | | | |
|---|---|---|---|---|---|---|---|---|
| Plasmid | | product/ | from/ | | | Restriction | | |
| ID | Gene/s | Description | Origin | Fwd Primer | Rev Primer | Enzymes | Vector | Inductor |
| pHD0119 | S. mutans | S. mutans | S. mutans | | | | pRGP-11 | — |
| | sccACDEFG | ΔsscB- | Xc47 chromosomal | | | | | |
| | | pRGP-12 | DNA sccABCDEFG | | | | | |
| | | | with an insertion | | | | | |
| | | | in sccB (SccB_1-277) | | | | | |
| pHD0120 | S. mutans | S. mutans | S. mutans | | | | pRGP-12 | — |
| | sccABCDEFG | ΔsscC | Xc47 chromosomal | | | | | |
| | | | DNA sccABCDEFG | | | | | |
| | | | with an insertion | | | | | |
| | | | in sccC (SccB_1-160) | | | | | |
| pHD0131 | pBAD24 | Empty | pBAD24::ampR | | | | pBAD24 | Arabinose |
| | | vector | empty | | | | | |
| | | | vector | | | | | |
| pHD0136 | S. mutans | SccABCDEFG | S. mutans | | | | pRGP-1 | — |
| | sccABCDEFG | | Xc47 chromosomal | | | | | |
| | | | DNA sccABCDEFG | | | | | |
| pHD0139 | Ori 15A | Smu | pHD0136 | A102 | A103 | | Modified | — |
| | Erm | empty | | (TACCTCGAGGGCAAAGCCG | (TACGGATCCGTTATTTCCTC | | pRGP1 | |
| | | vector | | TTTTTCCATAGGCTCCGCCC) | CCGTTAAATAATAGATAAC) | | | |
| | | ΔsscABCDEFG | | SEQ ID NO: 47 | SEQ ID NO: 48 | | | |
| pHD0183 | gacB gfp | GFP- | S. pyogenes | A042 (AGACTCGAG | A125 | BamHI/ | pWaldoE | IPTG |
| | | tagged | MGAS505 | ATGCAGGATGTTTTTATCAT | (AGACTCGAGATGTTCATTTA | XhoI | | |
| | | GacB | complete | TGGTAGC) SEQ ID NO: 49 | AAAATAAAGCCTCGTAC) | | | |
| | | | genome | | SEQ ID NO: 50 | | | |
| | | | GenBank | | | | | |
| | | | NC_007297 | | | | | |
| | | | NCBI (2015) | | | | | |
| pHD0194 | gacB | GacB_M5005_- | S. pyogenes | A155 | A156 | EcoRI/ | pBAD24 | Arabinose |
| | | RS03100 | MGAS505 | (TCTGAATTCATGCAGGATG | (ACACTGCAGTTAATGTTCAT | PstI | | |
| | | | complete | TTTTTATCATTGGTAGC) | TTAAAAATAAAGCCTCGTAC) | | | |
| | | | genome | SEQ ID NO: 51 | SEQ ID NO: 52 | | | |
| | | | GenBank | | | | | |
| | | | NC_007297 | | | | | |
| | | | NCBI (2015) | | | | | |
| pHD02227 | gacB_D126A | GacB | pHD0194 | A198 | (CAATCCAGCTGGGTTAGAG | EcoRI/ | pBDAD24 | Arabinose |
| | | amino | | (CACTCTAACCCAGCTGGAT | TGGAAACGGTCT) SEQ ID | PstI | | |
| | | acid | | TGATAAAAAAGCG) A199 | NO: 54 | | | |
| | | substitution | | SEQ ID NO: 53 | | | | |
| | | D126A | | | | | | |
| pHD0228 | gacB_E222A | GacB | pHD0195 | A200 | A201 | EcoRI/ | pBDAD24 | Arabinose |
| | | amino | | (CGTAATTATTTGCAGGAACA | (CGCTTTGTTCCTGCAAATAA | PstI | | |
| | | acid | | AAGCGTCCTAAATG) SEQ | TTACGAAACCGC) SEQ ID | | | |
| | | substitution | | ID NO: 55 | NO: 56 | | | |
| | | E222A | | | | | | |
| pHD0229 | gacB_D160A | GacB | pHD0196 | A202 | A203 | EcoRI/ | pBDAD24 | Arabinose |
| | | amino | | (CAATGCCAATATTAGCTGAA | (GGTCATTTCAGCTAATATTG | PstI | | |
| | | acid | | ATGACCAAATC) SEQ ID | GCATTGACCGC) SEQ ID | | | |
| | | substitution | | NO: 57 | NO: 58 | | | |
| | | D160A | | | | | | |
| pHD0230 | gacB_Y182A | GacB | pHD0197 | A204 | A205 | EcoRI/ | pBDAD24 | Arabinose |
| | | amino | | (GTCTGCGTTCCAGCAGCAA | (GTTTTATTGCTGCTGGAACG | PstI | | |
| | | acid | | TAAAACATGTTTTAG) SEQ ID | CAGACACAACCTTC) SEQ ID | | | |
| | | substitution | | NO: 59 | NO: 60 | | | |
| | | Y182A | | | | | | |
| pHD0231 | gacB_D126A | GacB | pHD0198 | A219 | A220 | EcoRI/ | pBDAD24 | Arabinose |
| | | amino | | (CTCTAACCCGTTTGGATTG | (CGCTTTTTTATCAATCCAAA | PstI | | |
| | | acid | | ATAAAAAAGCGTCCACCTCG) | CGGGTTAGAGTGGAAACGG | | | |
| | | substitution | | SEQ ID NO: 61 | TC) SEQ ID NO: 62 | | | |
| | | D126N | | | | | | |
| pHD0232 | gacB_E222Q | GacB | pHD0199 | A221 | A222 (GTTCCTCAAAATAATTA | EcoRI/ | pBDAD24 | Arabinose |
| | | amino | | (GGTTTCGTAATTATTTTGAG | CGAAACCGC) SEQ ID NO: 64 | PstI | | |
| | | acid | | GAACAAAGCG) SEQ ID | | | | |
| | | substituion | | NO: 63 | | | | |
| | | E222Q | | | | | | |
| pHD0233 | gacB_D160N | GacB | pHD0200 | A223 | A224 | EcoRI/ | pBDAD24 | Arabinose |
| | | amino | | (TGCCAATATTATTTGAAATG | (GATTTGGTCATTTCAAATAA | PstI | | |
| | | acid | | ACCAAATCAGCC) SEQ ID | TATTGGCATTGACCGCTACC) | | | |
| | | substitution | | NO: 65 | SEQ ID NO: 66 | | | |
| | | D160N | | | | | | |
| pHD0234 | gacB_Y182F | GacB | pHD0201 | A225 | A2226 | EcoRI/ | pBDAD24 | Arabinose |
| | | amino | | (GGTTGTGTCTGCGTTCCGA | (GTTTATTGCTTTCGGAACG | PstI | | |
| | | acid | | AAGCAATAAAACATGTTTTA | CAGACACAACCTTCACG | | | |
| | | substitution | | GACC) SEQ ID NO: 67 | SEQ ID NO: 68 | | | |
| | | Y128F | | | | | | |
| pHD0235 | gacB_K131R | GacB | pHD0202 | A241 | A242 | EcoRI/ | pBDAD24 | Arabinose |
| | | amino | | (TTTAGACCGCGTCCACTCT | (AGAGTGGACGCGGTCTAAA | PstI | | |
| | | acid | | AACCCGTCTGG) SEQ ID | TGGTCAAGACC) SEQ ID | | | |
| | | substitution | | NO: 69 | NO: 70 | | | |
| | | K131R | | | | | | |
| pHD0256 | S. pyogenes | GacA- | | A170 | A156 | Ncol/ | Modified | — |
| | gacA, | CDEFG | | (TTCGGATCCAACTATTAGC | (ACActgcagttaatgttcattt | PstI | pRGP1 | |
| | gacB-292-385, | from | | CTACATTCGAGAACAGG) | aaaaataaagcctcgtac) | | | |
| | gacCDEFG | S. pyogenes | | | SEQ ID NO: 72 | | | |
| | | MGAS505 | | | | | | |
| | | NC_007297 | | | | | | |
| | | with | | | | | | |
| | | GacB | | | | | | |
| | | 292-385 | | | | | | |
| | | (inactive) | | | | | | |
| pHD0312 | gacB_119- | GacB | pHD0183 | A015 | A016 | XhoI/ | pWaldoE | Arabinose |
| | 385 | without | | (CTTTAAGAAGGAGACTCGA | (GTCTGGATTGATAAAAAAGC | BamHI | | |
| | | residues | | GATGGGACGCTTTTTTATCA | GTCCCATCTCGAGTCTCCTT | | | |
| | | 1-118 | | ATCCAGAC) SEQ ID NO: 73 | CTTAAAG) SEQ ID NO: 74 | | | |
| pHD0313 | gacB_127- | GacB | pHD0183 | A017 | A018 | XhoI/ | pWaldoE | Arabinose |
| | 385 | without | | (CTTTAAGAAGGAGACTCGA | (GACCGTTTCCACTCTAACC | BamHI | | |
| | | residues | | GATGGGGTTAGAGTGGAAA | CCATCTCGAGTCTCCTTCTT | | | |
| | | 1-127 | | CGGTC) SEQ ID NO: 75 | AAAG) SEQ ID NO: 76 | | | |
| pHD0322 | gacB_76- | GacB | pHD0194 | A3464 | A156 | BamHI/ | pBAD24 | Arabinose |
| | 385 | without | | (GGATCCATGATGGCAATTA | (ACACTGCAGTTAATGTTCAT | PstI | | |
| | | residues | | CCTATGCCCTGTC) SEQ ID | TTAAAAATAAAGCCTCGTAC) | | | |
| | | 1-76 | | NO: 77 | SEQ ID NO: 78 | | | |
| pHD0323 | gacB_23- | GacB | pHD0194 | A365 | A156 | BamHI/ | pBAD24 | Arabinose |
| | 385 | without | | (GGATCCATGGAAGAGTTGA | (ACACTGCAGTTAATGTTCAT | PstI | | |
| | | residues | | TTAGTCATCAATCATCT) | TTAAAAATAAAGCCTCGTAC) | | | |
| | | 1-23 | | SEQ ID NO: 79 | SEQ ID NO: 80 | | | |
| pHD0332 | sccB_TTG | Extended | pHD0136 | A373 | A370 | KpnI/ | pBAD2 | Arabinose |
| | | SccB_TTG_BAA | | (GGTACCATGCGTCATATATT | (ATATTCTAGAATTATAGGTA | PstI | | |
| | | 32089.1 | | CATCATAGGAAGTCGCG) | CCCCTTATTAAAGTTAAACAA | | | |
| | | with a | | SEQ ID NO: 81 | AATTATTTC) SEQ ID NO: 82 | | | |
| | | TTG | | | | | | |
| | | start | | | | | | |
| | | codon | | | | | | |
| pHD0333 | sccB_ATG | Extended | pHD0136 | 1ST A425 | 1ST A426 | KpnI/ | pBAD24 | Arabinose |
| | | SccB_TTG_BAA | | (GCTATCCGTGAGTTCATGA | (CGAAGTCATGAACTCACGG | PstI | | |
| | | 32089.1 | | CTTCG) SEQ ID NO: 83 | ATAGC). 2ND A0424 SEQ ID | | | |
| | | with a | | 2ND A0372 | NO: 85 | | | |
| | | ATG | | (CTGCAGTTAACTTTCATGTA | (GGAGGAATTCACCTTGCGT | | | |
| | | start | | AGAACAAGTCCTCGTAC) | CATATATTCATCATAGGAAG | | | |
| | | codon | | SEQ ID NO: 84 | TCGCG) SEQ ID NO: 86 | | | |
| pHD0440 | wchF_1- | WchF- | pHD0194-pHD0486 | A634 | A768 | EcoRI/ | pBAD24 | Arabinose |
| | 186 + | GacB | | (TCTGAATTCATGAAACAGTC | (GGTTGTGTCTGCGTTCCAT | PstI | | |
| | gacB_179- | chimaera | | AGTTTATATCATTGGTTCAA) | AAGCAATAAAGGTCGTCTTG | | | |
| | 385 | | | SEQ ID NO: 87 | GGCTGATACTG) SEQ ID | | | |
| | | | | | NO: 88 | | | |
| pHD0441 | gacB_L128H_R131L_- | GacB | pHD605 | A770 | A771 | EcoRI/ | pBAD24 | Arabinose |
| | GNT100ACR_A105P | with | | (CCAGATTCAGAACCCTATTT | (CGATTGTGAATCTGCTTCAC | PstI | | |
| | | amino | | TTTATGTGTTGGCGTGTCGA | AAATGGCGCAATAAATGGGC | | | |
| | | substitution | | GTAGGCCCATTTATTGCGCC | CTACTCGACACGCCAACACA | | | |
| | | L128H_R131L_- | | ATTTGTGAAGCAGATTCACA | TAAAAAATAGGGTTCTGAAT | | | |
| | | GNT100ACR_A105P | | ATCG) SEQ ID NO: 89 | CTGG) SEQ ID NO: 90 | | | |
| pHD0445 | gacBL128H_R131L | GacB | pHD0194 | A736 | A737 | EcoRI/ | pBAD24 | Arabinose |
| | | with | | (CAATCCAGACGGGCACGGAG | (GTCTTGACCATTTAGACAGT | PstI | | |
| | | amino | | TGGAAACTGTCTAATGGTC | TTCCACTCGTGCCCGTCTGG | | | |
| | | acid | | AAGAC) SEQ ID NO: 91 | ATTG) SEQ ID NO: 92 | | | |
| | | substitutions | | | | | | |
| | | L128H_R131L | | | | | | |
| pHD0457 | gacB_D160N - | GFP- | pHD0233 | A223 | A224 | XhoI/ | pWaldoE | IPTG |
| | gfp | tagged | | (TGCCAATATTATTTGAAATG | (GATTTGGTCATTTCAAATAA | BamHI | | |
| | | GacB | | ACCAAATCAGCC) SEQ ID | TATTGGCATTGACCGCTACC) | | | |
| | | amino | | NO: 93 | SEQ ID NO: 94 | | | |
| | | acid | | | | | | |
| | | substitution | | | | | | |
| | | D160N | | | | | | |
| pHD0458 | gacB_Y182D - | GFP- | pHD0234 | A225 | A226 | XhoI/ | pWaldoE | IPTG |
| | gfp | tagged | | (GGTTGTGTCTGCGTTCCGA | (GTTTTATTGCTTTCGGAACG | BamHI | | |
| | | GacB | | AAGCAATAAAACATGTTTTA | CAGACACAACCTTCACG) | | | |
| | | amino | | GACC) SEQ ID NO: 95 | SEQ ID NO: 96 | | | |
| | | acid | | | | | | |
| | | substitution | | | | | | |
| | | Y182F | | | | | | |
| pHD0477 | S. dysgalactiae | GacB | NCBI | A604 | A605 | PstI/ | pBAD24 | Arabinose |
| | subsp. | homolog | NC_0175671.1 | (ATCTGAATTCATGCAGGAT | (ACACTGCAGTTAATGTTCAT | EcoRI | | |
| | equisimilis | from the | WP_01461218.01 | GTTTTCATCATTGGTAGC) | CTAAAAATAAAGCCTCATAC) | | | |
| | ATCC | Group G | | SEQ ID NO: 97 | SEQ ID NO: 98 | | | |
| | 12394_RS03945 | Streptococcus - | | | | | | |
| | | SDSE_ATC12394_- | | | | | | |
| | | RS03945 | | | | | | |
| pHD0478 | S. agalactiae | GacB | NCBI: | A606 | A607 | PstI/ | pBAD24 | Arabinose |
| | SAG1423 | homolog | txid208435 | (TCTgaattcatgcaagatgttttc | (ACActgcagttaactttcGttCaaG | EcoRI | | |
| | | from the | WP_001154381.1 | attatagg) SEQ ID NO: 99 | aacaaGtcctc) SEQ ID NO: 100 | | | |
| | | Group B | | | | | | |
| | | Streptococcus - | | | | | | |
| | | KXA41920.1 | | | | | | |
| pHD0479 | S. dysgalactiae | GacB | GenBank: | A607 | A609 | PstI/ | pBAD24 | Arabinose |
| | subsp. | homolog | AP012976. | (ATGAATTCATGCAGGATGTT | TAAAAATAAAGCCTCATACT | EcoRI | | |
| | equisimilis | from the | BAN9325.1 | TTCATCATTGGTAGCAGA) | CCCCAACAAT) SEQ ID | | | |
| | 167 | Group C | | SEQ ID NO: 101 | NO: 102 | | | |
| | rgpAc | Streoptococcus - | | | | | | |
| | | WP_022554465.1 | | | | | | |
| pHD0486 | S. pneumonia | WchF_SBT85395.1 | CAI34122 | A634 | A635 | PstI/ | pBAD24 | Arabinose |
| | wchF | from | NCBI | (TCTgaattcatgaaacagtcagt | (ATATctgcaggcatcatacagta | EcoRI | | |
| | | S. pneumoniae | taxon: | ttatatcattggttcaa) | aacacttcctcataatctgac) | | | |
| | | serotype | 1313 | SEQ ID NO: 103 | SEQ ID NO: 104 | | | |
| | | 2 | | | | | | |
| pHD0605 | GacB_L128H_R131L_- | GacB | pHD0445 | A772 | A773 | EcoRI/ | pBAD24 | Arabinose |
| | GNT100ACR_mutant | with | | (CCAGATTCAGAACCCTATTT | (CGATTGTGAATCTGCTTCAC | PstI | | |
| | | amino | | TTTATGTGTTGgcgtgtcgaGTA | AAATGGCGCAATAAAagcGC | | | |
| | | acid | | GGCgctTTTATTGCGCCATTT | CTACtgacacgcCAACACATAA | | | |
| | | substitutions | | GTGAAGCAGATTCACAATCG) | AAAATAGGGTTCTGAATCTG | | | |
| | | L128H_R131L_- | | SEQ ID NO: 105 | SEQ ID NO: 106 | | | |
| | | GNT100ARC | | | | | | |

Determination of RhaPS Production

50 μL of OD₆₀₀-normalised overnight cultures grown at 37° C. were mixed with 50 μL of 6×SDS-loading buffer and resolved in 20% Tricine-SDS gels (29). Assessment of the RhaPS production was performed via immunoblotting on PVDF membranes following the traditional immunoblotting technique. Primary antibody: rabbit-raised anti-Streptococcus pyogenes Group A carbohydrate polyclonal antibody (Abcam, ab21034). Secondary antibody: goat-raised anti-rabbit IgG HRP conjugate (Biorad, 170-6515). Immunoreactive signals were captured using GENESYS™ 10S UV-Vis Spectrophotometer (Thermo Scientific) after exposure to the Clarity Western ECL (Biorad).

Extraction and Radiolabelling of Lipid-Linked Oligosaccharides

Radiolabelled lipid-linked saccharides (LLS) of induced E. coli CS2775 cells bearing the selected plasmids were extracted using 1:1 CHCl₃/CH₃OH and water-saturated butan-1-ol (1:1 v/v) solution to determine the addition of sugar residues in vivo after glucose D[6s³H] (N) (Perkin Elmer) supplementation (1 mCi/mL). The incorporated radioactivity was measured in a Beckman Coulter® LS6000SE scintillation counter. The organic phase containing the LLSs were normalised to 0.05 μCi/μL. The samples were separated via thin layer chromatography (TLC) on a HPTLC Silica Gel 60 plate (Merck) using a C:M:AC:A:W mobile phase (180 mL chloroform+140 mL methanol+9 mL 1M ammonium acetate+9 mL 13 M ammonia solution, 23 mL distilled water), then dried and sprayed with En 3 Hance™ liquid (Perkin Elmer). Radioautography images were obtained from Carestream® Kodak® BioMax® XAR Film and MS Intensifying Screens after 5 to 10 days.

Purification of Recombinantly Expressed Membrane Associated Proteins

The purification was conducted following the established protocol from Waldo, et. al. (30) with the following modifications. Overnight cultures of E. coli C43 (DE3) cells expressing C-terminal GFP-fusion proteins were diluted 1:100, incubated for 3 hours until OD₆₀₀=0.6, induced with 0.5 mM IPTG and shifted to room temperature overnight, all at 200 rpm shaking. GPF expression was detected through in-gel fluorescence using a Fujifilm FLA-5000 laser scanner. Cloning, expression and purification of GacB-WT, GacB-D160N-GFP and GacB-Y182-GFP: plasmids containing GFP-Hiss-tagged recombinant proteins were constructed as described in Table 1 into the vector pWaldo-E (30). For protein production and purification purposes, the vectors were transformed into E. coli C43 (DE3) cells and expressed as described above. The cells were fractionated using an Avestin C3 High-Pressure Homogenisator (Biopharma, UK) and spun down at 4000×g. Further centrifugation of the supernatant at 200 000×g for 2 h rendered 2-3 g of membrane containing the GacB-GFP proteins. Membranes were solubilised in Buffer 1 (500 mM NaCl, 10 mM Na₂HPO₄, 1.8 mM KH₂PO₄ 2.7 mM KCl, pH of 7.4, 20 mM imidazole, 0.44 mM TCEP) with the addition of 1% DDM (Anatrace) for 2 hr at 4° C. and bound to a 1 mL Ni-Sepharose 6 Fast Flow (GE healthcare) column, prewashed with buffer 1 plus 0.03% DDM. Elution was conducted using Buffer 1 supplemented with 250 mM imidazole and 0.03% DDM. Imidazole was removed using a HiPrep 26/10 desalting column (GE Healthcare) equilibrated with Buffer (PBS, 0.03% DDM, 0.4 mM TCEP). The GFP-His tag was removed with PreScission Protease cleavage in a 1:100 ratio overnight at 4° C. Cleaved GacB proteins were collected after negative IMAC. Protein identity and purity was determined by tryptic peptide mass fingerprinting, matrix-assisted laser desorption/ionisation time-of-flight mass spectrometry (MALDI-TOF), respectively (University of Dundee ‘Fingerprints’ Proteomics Facility).

Synthesis of Acceptor Acceptor 1 and 2

Acceptor 2 (P¹-(11-phenoxyundecyl)-P²-(2-acetamido-2-deoxy-α-D-glucopyranosyl) diphosphate) was synthesised as sodium salt from phenoxyundecyl dihydrogen phosphate and 2-acetamido-2-deoxy-3,4,6-tri-O-acetyl-α-D-glucopyranosyl dihydrogen phosphate according to the procedure by T. N. Druzhinina et al. 2010 (94). Acceptor 1 (P¹-tridecyl-P²-(2-acetamido-2-deoxy-α-D-glucopyranosyl) diphosphate) was synthesised from tridecyl dihydrogen phosphate (obtained similarly to phenoxyundecyl dihydrogen phosphate) by the same procedure as described for acceptor 2.

GacB In Vitro Enzymatic Reaction

Purified GacB-WT-GFP, GacB-D160N-GFP, GacB-Y182F-GFP and the GacB (tag-less) protein (0.15 mg/ml final concentration) were mixed in a 100 μl TBS buffer supplemented with 1 mM TDP-Rha as sugar donor and 1 mM acceptor-1 (C₁₃—PP-GlcNAc) or 1 mM acceptor-2 (Phenol-O—C₁₁H₂₂—PP-GlcNAc) as acceptor substrate. The reaction was incubated for 3 h to 24 h at 30° C. The assay mixture was adjusted with the exchange of the nucleotide sugar donor to UDP-Rha or UDP-GlcNAc and with the addition of either 1 mM MgCl₂, 1 mM MnCl₂, or 1 mM EDTA to define the essentiality of metal dependency.

Mass Spectrometry Analysis

Matrix-assisted Laser Desorption Ionization Time-of-Flight (MALDI-TOF) was used to analyse the acceptors and products of the GacB in vitro assay. 100 μl reaction samples were purified over a 100 μL Sep-Pak C18 cartridges (Waters, UK), pre-equilibrated with 5% EtOH. The bound samples were washed with 800 μl H₂O and 800 μl 15% EtOH, eluted in two fractions with a) 800 μl 30% and b) 800 μl 60% EtOH. The two elution fractions were dried in a speed vac and resuspended in 20 μl 50% MeOH. 1 μl of sample was mixed with 1 μl 2,5-dihydroxybenzoic (DHB) acid matrix (15 mg/mL in 30:70 acetonitrile: 0.1% TFA) and 1 μl was added to the MALDI grid. Samples were analyzed by MALDI in an Autoflex speed mass spectrometer set up in reflection positive ion mode (Bruker, Germany).

NMR Analysis

The purified GacB in vitro assay products (0.5-2 mg) were dissolved in D20 (550 μL) and measured at 300 K. The spectra were acquired on a 4-channel Avance III 800 MHz Bruker NMR spectrometer equipped with a 5 mm TCl CryoProbe™ with automated matching and tuning. 1D spectra were acquired using the relaxation and acquisition times of 5 and 1.8 s, respectively. Between 32 and 512 scans were acquired using the spectral width of 11 ppm. J connectivities were established in a series of 1D and 2D TOCSY experiments with mixing times between 20 and 120 ms. Selective 1D TOCSY spectra (32) were acquired using a 40 ms Gaussian pulses and DIPSI-2 sequence (33) (γB₁/2π=10 kHz) for spin lock of between 20 and 120 ms. The following parameters were used to acquire 2D TOCSY and ROESY experiments: 2048 and 768 complex points in t₂ and t₁, respectively, spectral widths of 11 and 8 ppm in F₂ and F₁, yielding t₂ and t₁ acquisition times of 116 and 60 ms, respectively. Sixteen scans were acquired for each t₁ increments using a relaxation time of 1.5 s. The overall acquisition time was 6-7 hours per experiment. A forward linear prediction to 4096 points was applied in F₁. A zero filling to 4096 was applied in F₂. A cosine square window function was used for apodization prior to Fourier transformation in both dimensions. The ROESY mixing time was applied in the form of a 250 ms rectangular pulse at γB₁/2π=4167 Hz. DIPSI-2 sequence (γB₁/2π=10 kHz) was applied for a 20, 80 and 120 ms spin lock. 2D magnitude mode HMBC experiments: 2048 and 128 complex points in t₂ and t₁, respectively, spectral widths of 6 and 500 ppm in F₂ and F₁, yielding t₂ and t₁ acquisition times of 0.35 s and 0.6 ms, respectively. Two scans were acquired for each of 128 t₁ increments using a relaxation time of 1.2 s. The overall acquisition time was 8 minutes. A forward linear prediction to 512 points was applied in F₁; zero filling to 4096 was applied in F₂. A sine square window function was used for apodization prior to Fourier transformation in both dimensions.

GacC/Homologous Enzymes Protein Purification

For production of recombinant proteins, target genes (GacC, GbcC, Cps2F, SccC) were synthesized using IDT's gBlock gene fragment synthesis service. Wild-type sequences for GacC and its' homologs were PCR amplified with overhangs designed for cloning into pOPINF¹, which contains an N-terminal 6× Histidine tag for affinity purification. Cloning into pOPINF was carried out using In-Fusion™ cloning technology (Clontech). The resulting plasmids were then transformed into DH5α: competent cells for propagation and extraction (miniprep kit; Qiagen). Positively transformed plasmids were identified by size comparison to a non-transformed control pOPINF plasmid using gel electrophoresis, which were subsequently confirmed by DNA sequencing. For insertion of point mutants, wild-type plasmids were used as templates to PCR amplify 2 overlapping fragments containing the desired point mutant. Fragments were designed to contain a minimum of a 15 bp overlap and were cloned into pOPINF and sequence verified as for wild type plasmids. A full list of primers used for both wild-type and mutant cloning can be found in Table A.

Sequence verified plasmids were then transformed into C43 cells for protein expression. For activity assays, 1 L of E. coli culture typically yielded enough protein for >50 assays (1 mg L⁻¹). Cultures were grown at 37° C. and shaking at 200 RPM to an OD of 0.6-1, at which point they were transferred to 18° C. for 1 hour before induction with 0.5 mM isopropyl β-D-thiogalactopyranoside (IPTG). Cultures were left shaking at 18° C. overnight. Following centrifugation of the culture at 3000×g, proteins were extracted in Buffer A0 (50 mM HEPES pH 7.5, 300 mM NaCl, 10% glycerol, 2 mM TCEP) supplemented with protease inhibitors, using an Avestin C3 cell disruptor according to the manufacturer's instructions. Lysed cultures were then subject to ultracentrifugation at 200,000×g and the supernatant was collected. The supernatant containing the soluble proteins of interest was then purified over a Nickel-affinity (Thermo Fisher) column using wash Buffer A (50 mM HEPES pH 7.5, 300 mM NaCl, 10% glycerol, 2 mM TCEP, 20 mM imidazole) and elution Buffer B (50 mM HEPES pH 7.5, 300 mM NaCl, 10% glycerol, 2 mM TCEP, 400 mM imidazole) according to manufacturer's instructions. Elution fractions containing the target proteins were then passed over a desalting column, preequilibrated with Buffer A0, to remove imidazole. Protein samples were concentrated to 0.5-1 mg/ml and snap frozen in liquid nitrogen until use.

TABLE A

| Name | SEQUENCE (5′ TO 3′) | Use |
|---|---|---|
| A872_GacC_pOP1N_fwd | AAGTTCTGTTTCAGGGCCCGAACATTAAT | Cloning of N- |
| | ATTTTACTATCCACCTAC (SEQ ID NO: | terminal region of |
| | 107) | GacC constructs |
| A873_GacC_pOP1N_rev | ATGGTCTAGAAAGCTTTACTTTCTCCTGT | Cloning of C- |
| | AACCAAATAAGGTAAC (SEQ ID NO: 108) | terminal region of |
| | | GacC constructs |
| A810_GbcC_pOP1N_fwd | AAGTTCTGTTTCAGGGCCCGAAGGTTAAT | Cloning of N- |
| | ATCTTAATGGCCACCTAC (SEQ ID NO: | terminal region of |
| | 109) | GbcC constructs |
| A811_GbcC_pOP1N_rev | ATGGTCTAGAAAGCTTTATCTCTTATTGTA | Cloning of C- |
| | ATAATTTGTTGCAATCAACC (SEQ ID NO: | terminal region of |
| | 110) | GbcC constructs |
| A948_RgpB_pOP1N_fwd | AAGTTCTGTTTCAGGGCCCGAAAGTTAAT | Cloning of N- |
| | ATTTTAATGTCCACCTAC (SEQ ID NO: | terminal region of |
| | 111) | SccC constructs |
| A949_RgpB_pOP1N_rev | ATGGTCTAGAAAGCTTTATTTTCTCCTATA | Cloning of C- |
| | ACCAAATTTAG (SEQ ID NO: 112) | terminal region of |
| | | SccC constructs |
| A936_Cps2F_pOPIN_fwd | AAGTTCTGTTTCAGGGCCCGAGTAACAA | Cloning of N- |
| | GCAAATTG (SEQ ID NO: 113) | terminal region of |
| | | Cps2F constructs |
| A937_Cps2F_pOPIN_rev | ATGGTCTAGAAAGCTTTAAATAAACATTAA | Cloning of C- |
| | CTCACCG (SEQ ID NO: 114) | terminal region of |
| | | Cps2F constructs |
| A968_GbcC_R217G_nterm_rev | CTTAAATCTCTTATCCATTGTACCCGCCC | Reverse primer for |
| | CCAAAAC (SEQ ID NO: 115) | N-terminal fragment |
| | | of R217G |
| A969_GbcC_R217G_cterm_fwd | GTTTTGGGGGCGGGTACAATGGATAAGA | Forward primer for |
| | GATTTAAG (SEQ ID NO: 116) | C-terminal fragment |
| | | of R217G |
| A970_GbcC_K221G_nterm_rev | CGAAGTATCTTAAATCTACCATCCATTGT | Reverse primer for |
| | CCTC (SEQ ID NO: 117) | N-terminal fragment |
| | | of K221G |
| A971_GbcC_K221G_cterm_fwd | GAGGACAATGGATGGTAGATTTAAGATAC | Forward primer for |
| | TTCG (SEQ ID NO: 118) | C-terminal fragment |
| | | of K221G |
| A972_GbcC_K224G_nterm_rev | GACCTTCACGAAGTATACCAAATCTCTTA | Reverse primer for |
| | TCC (SEQ ID NO: 119) | N-terminal fragment |
| | | of K224G |
| A973_GbcC_K224G_cterm_fwd | GGATAAGAGATTTGGTATACTTCGTGAAG | Forward primer for |
| | GTC (SEQ ID NO: 120) | C-terminal fragment |
| | | of K224G |
| A958_GbcC_R227G_nterm_rev | TAGATTTAGGACCTTCACCAAGTATCTTA | Reverse primer for |
| | AATCTC (SEQ ID NO: 121) | N-terminal fragment |
| | | of R227G |
| A959_GbcC_R227G_cterm_fwd | GAGATTTAAGATACTTGGTGAAGGTCCTA | Forward primer for |
| | AATC (SEQ ID NO: 122) | C-terminal fragment |
| | | of R227G |
| A992_GacC_D91A_Fwd | GCAGATGTCTATTTTTTCAGTGCCCAAGA | |
| | TGATATATGGTTAGAC (SEQ ID NO: 123) | |
| A993_GacC_D91A_rev | GTCTAACCATATATCATCTTGGGCACTGA | |
| | AAAAATAGACATCTGC (SEQ ID NO: 124) | |
| A994_Y206F_fwd | CTTGATATTCCAACAGAATTATTCCGTCA | |
| | GCACGATGC (SEQ ID NO: 125) | |
| A995_Y206F_rev | GCATCGTGCTGACGGAATAATTCTGTTGG | |
| | AATATCAAG (SEQ ID NO: 126) | |
| A998_GacC_H209A_fwd | CAACAGAATTATACCGTCAGGCCGATGCT | |
| | AACGTGTTGGG (SEQ ID NO: 127) | |
| A999_GacC_H209A_rev | CCCAACACGTTAGCATCGGCCTGACGGT | |
| | ATAATTCTGTTG (SEQ ID NO: 128) | |

¹Berrow NS, Alderton D, Sainsbury S, Nettleship J, Assenberg R, Rahman N, Stuart DI, Owens RJ. A versatile ligation-independent cloning method suitable for high-throughput expression screening applications. Nucleic acids research. 2007 Mar 1; 35(6): e45.

HPLC Assay

For in vitro enzyme analyses, 50 μl reactions were set up to include 2.5 mM synthetic lipid acceptor PH—O—C₁₁H₂₂—PP-alpha-NAG, 12.5 mM TDP-L-rhamnose, 0.5-1.5 μM GacB-GFP, and 1.25-2.5 μM GacC or homolog/mutant of interest, topped up to 50 μl with TBS Buffer supplemented with 2 mM MnCl₂. Reactions were incubated at 30° C. and when desired timepoints were met, quenched with 50 μl acetonitrile and left on ice for 15 minutes. Reactions were spin filtered at 14,000 RPM in a benchtop centrifuge to remove precipitated protein before being injected onto a Xbridge BEH Amide OBS Prep column (130 Å, 5 μM, 10×250 mm) connected to an HPLC system fitted with a UV detector set to 270 nm (Ultimate 3000, Thermo). Samples were applied to the column at 4 ml/min using Running Buffer A (95% acetonitrile, 10 mM ammonium acetate, pH 8) and Running Buffer B (50% acetonitrile, 10 mM ammonium acetate, pH 8) over a gradient of increasing concentration of B. Increasingly polar products with additional sugar residues eluted later into the gradient, with the triple rhamnosylated GacC product typically eluting ˜14 min into a 36 min run. Products purified from the HPLC were dried in a speed vacuum to remove excess acetonitrile, before being freeze-dried to remove residual water and ammonium acetate. Samples could be stored at −20° C. for structural analysis.

NMR Analysis GacC Product

For NMR analysis at the University of Dundee, HPLC purified products (0.5-2 mg) were resuspended in 600 μl of D20 and NMR spectra were recorded at 293 K. The spectra were acquired on a Bruker AVANCE III HD 500 MHz NMR Spectrometer equipped with a 5-mm QCPI cryoprobe. NMR spectra were recorded as described for the GacB reaction product. Spectra were analysed using Bruker Topsin (4.0.7).

Results

GacB is Required for the Biosynthesis of the GAC RhaPS Chain

To investigate the GacB function and to identify potential catalytic residues, we used E. coli as a heterologous expression system to study the GAC RhaPS backbone biosynthesis. We constructed two vectors carrying the homologous genes from S. pyogenes, gacACDEFG (gacA-G; ΔgacB) and gacB (FIG. 1A).

The RhaPS chain is presumed to be translocated to the outer membrane in E. coli, which naturally contains rhamnose attached to the lipopolysaccharides. Thus, to avoid unspecific binding of the anti-GAC antibody, all transformations were made using a rfaS-deficient strain (20). The interruption of the rfaS gene impedes the attachment of rhamnose to the LPS on the bacterial outer membrane, rendering a strain that lacks endogenous rhamnose on its surface (20). The role of GacB was investigated using the traditional complementation strategy depicted in FIG. 1.

We investigated the production of RhaPS by gacA-G from our complementation approach using immunoblots of total cells lysates (FIG. 1B). If the expression of GacBCDEFG is sufficient to produce the RhaPS chain, then we should be able to detect the synthesised RhaPS using a specific anti-GAC antibody. The results showed that E. coli cells lacking the gacA-G gene cluster (empty vector) did not produce RhaPS (FIG. 1, lane 2). Likewise, transformants bearing the ΔgacB or ΔsccB plasmids lost reactivity with the GAC antibody (FIG. 1, lane 3 and 5). Instead, co-transformation of sccB+ΔsccB or gacB+ΔgacB restored the RhaPS production, underlining the essentiality of sccB and gacB for the biosynthesis of the GAC backbone (FIG. 1, lane 4 and 6).

In order to investigate if GacB and SccB are catalysing the same reaction, we tested the ability of GacB to functionally substitute SccB and vice versa by co-transforming ΔsccB+gacB and ΔgacB+sccB. In all cases, SccB and GacB were interchangeable (FIG. 2). GacB's predicted initiation codon was different from S. mutans SccB, with the latter using TTG instead of ATG (FIG. 2). We decided to test two versions of SccB; one with a TTG as the initiation codon and the other one with an ATG. Both versions rendered an active enzyme that could complement either ΔsccB and ΔgacB (FIG. 2). Unless stated otherwise, all further work was conducted using sccB constructs with the native TTG start codon.

GacB Extends a Lipid-Linked Precursor

We investigated whether GacB is a GT that uses GlcNAc-PP-Und as an acceptor. We performed an in vivo experiment generating radiolabelled lipid-linked oligosaccharides (LLO), which were isolated from the bacterial membrane and separated via thin-layer chromatography (TLC). Based on the annotation as a rhamnosyltransferase, radiolabelled dTDP-β-L-rhamnose would be the preferred sugar donor for GacB. However, this compound is not commercially available, therefore tritiated glucose was chosen as an alternative. Inside the bacterial cell, glucose is used as a substrate to synthesise a wide array of organic components, including dTDP-L-rhamnose (25).

We hypothesised that GacB transfers an activated sugar from a (radiolabelled) nucleotide sugar donor to a membrane-bound acceptor monosaccharide-PP-Und, e.g. GlcNAc-PP-Und. Therefore, we expected a change in size of the membrane bound acceptor, compared to the signal of the monosaccharide lipid-linked acceptor after running the samples in a TLC plate. As negative control, we used E. coli CS2775 (ArfaS) transformed with the empty vector. This transformant showed a signal consistent with the generation of monosaccharide-PP-Und (FIG. 3 lane 1). Upon expression of either the gacB or sccB genes, we observed the accumulation of a radioactive signal that migrated more slowly on the TLC plate, suggesting a higher molecular mass for these compounds (FIG. 3, lane 3 and 4). The same shift was observed for the sccAB-DEFG (AsccC) construct (FIG. 3, lane 2), demonstrating that sccB and gacB can glycosylate a lipid-linked precursor. Based on the literature, we assume that the upper radiolabelled band corresponds to GlcNAc-PP-Und, and the lower one to Rha-GlcNAc-PP-Und (8, 9).

GacB is a Rhamnosyltransferase that Transfers Rhamnose from TDP-β-I-Rha onto GlcNAc-PP-Lipid Acceptors

The observed band shift suggested that GacB adds a monosaccharide to a lipid-linked precursor, most likely GlcNAc-PP-Und. We investigated this hypothesis using recombinantly produced and purified GacB WT and amino acid mutants (mutants D₁₆₀N and Y₁₈₂F). We established an in vitro assay using the predicted nucleotide sugar donor, TDP-β-L-rhamnose and a synthetic acceptor substrate. We tested two of these synthetic substrates designed to mimic the native lipid-linked acceptor: C₁₃H₂₇—PP-GlcNAc (acceptor 1) or phenyl-O—C₁₁H₂₂—PP-GlcNAc (acceptor 2) (FIG. 7C). The reactions were purified and characterised using matrix-assisted laser desorption ionisation mass spectrometry (MALDI-MS) in positive ion mode.

The MALDI-MS spectra of the enzymatic reaction (FIG. 4) confirmed that GacB catalyses the addition of one rhamnose to both acceptor substrates when incubated with TDP-β-L-rha (FIGS. 4B and E). Acceptor 1 possesses a molecular weight of 563 Da and is detected at both m/z=608 [M-1H+2Na]⁺ and m/z=630 [M-2H+3Na]⁺ (FIG. 4A). GacB-GFP and GacB lacking the GPF tag modified the acceptor, resulting in one predominant peak at m/z=776 [M-2H+3Na]⁺ (FIG. 4B, C). In this spectrum, we can also observe an additional peak of lower intensity at m/z=754 [M-1H+2Na]⁺, corresponding to the modified acceptor 1 coupled with 2 Na⁺ ions, instead of 3 Na⁺ ions. In both cases, the products are shifted by m/z=146 compared to the unmodified acceptor, which is consistent with the addition of one rhamnose via a glycosidic linkage. The same mass shift was observed for the second acceptor; the peaks of the unmodified acceptor 2 (FIG. 4D) were detected at m/z=672 [M-1H+2Na]⁺ and m/z=694 [M-2H+3Na]⁺, while the product peaks emerge at m/z=818 [M-1H+2Na]⁺ and m/z=840 [M-2H+3Na]⁺ (FIGS. 4E and 4F). We also tested the ability of GacB to catalyse the rhamnosylation of GlcNAc-α-1-P, but the reaction rendered no detectable product (data not shown), suggesting that the enzyme interacts not only with the GlcNAc-P, but might require the second phosphate and the lipid component to recognise the acceptor substrate.

We further investigated GacB's specificity towards the sugar-nucleotide donor. In particular, we tested if GacB is selective for thymidine-based nucleotides and tolerates uridine-based nucleotides such as UDP-Glc, UDP-GlcNAc and UDP-Rha. As shown before, in the presence of TDP-β-L-Rha, two products consistent with the incorporation of rhamnose plus either two or three sodium cations were observed in the spectrum (FIG. 5A). In contrast, no product peaks were observed with UDP-α-D-Glc or UDP-α-D-GlcNAc as substrates (FIGS. 5B and C), while residual activity was detected for UDP-β-L-Rha (FIG. 5D). This data demonstrate that GacB does not tolerate α-D configured nucleotide sugars. Furthermore, GacB has specificity towards the deoxyribose (TDP-rhamnose) and/or requires binding of the thymine methyl group.

Finally, we assessed metal ion dependency in vitro. Compared to the control reaction (FIG. 6B), we noticed no significant differences in the rhamnosylation activity of the enzyme when GacB was supplemented with MgCl₂, MnCl₂ or EDTA as a metal chelator (FIG. 6C, D, E), indicating that GacB does not require a divalent metal ion for its activity.

Together, these data confirmed our previous conclusions drawn from the LLSs radiolabelled assay (FIG. 3). This is the first in vitro evidence revealing that GacB is a metal-independent rhamnosyltransferase that catalyses the initiation step in the GAC RhaPS backbone biosynthesis by transferring a single rhamnose to GlcNAc-PP-Und using TDP-β-L-Rha as the exclusive activated nucleotide sugar donor.

Investigation of GacB's Catalytic Residues

We were unable to obtain diffraction-quality crystals from the detergent-extracted protein, which would ultimately have revealed detailed insights into the catalytic region. We constructed a GacB structural model based on two enzymes that belong to the GT-4 family of GTs: Bacillus anthracis' BaBshA (PDB entry 3mbo) (72) and Corynebacterium glutamicum's MshA (PDB ID: 3c4v) (24). BaBshA shares 15% identity in 64 out of 424 amino acids. MshA is a ‘homologous’ GT that shares 16% identical residues in a sequence stretch of 71 residues out of 446. Based on the scarce information provided by the structural models and the multiple sequence alignment described in detail below, we mutated several residues that are highly conserved in over forty pathogenic streptococci species.

Our in vitro E. coli system is the first one that enables the study of GacB mutant proteins, allowing the identification of those mutants that abrogate or reduce the production of RhaPS backbone. Conducting this in S. pyogenes is not possible since deletion of the gacB gene renders inviable cells (1, 20). We used the information available from the GT models mentioned above and the sequence alignment of multiple streptococci to select residues that might be involved in substrate binding, which tends to be conserved among GT. Through in-situ mutagenesis, we constructed nine recombinant versions of GacB containing the following amino acid substitutions: D126A, D126N, E222A, E222Q, D160A, D160N, Y182A, Y182F and K131R. The latter mutation was included as a negative control since it is a conserved predicted surface residue that presumably is not engaged in the catalytic activity or could inactivate the enzyme otherwise.

We found that substitution of D160 with an asparagine led to a drastic reduction in the production of the RhaPS chain, while an alanine residue did not cause such significant effect. This suggests that the D160 carboxyl group might be required for catalysis, which potentially can be replaced in the alanine mutant by a water molecule. A more severe effect was observed with mutations of Y182. The alanine substitution of Y182 (Y182A) impeded the RhaPS backbone biosynthesis significantly, while Y182F completely inactivated GacB, suggesting an essential role for the Y182 hydroxyl group in GacB's enzymatic activity.

We further investigated the mutants D160N and Y182F in an in vitro assay using recombinantly expressed and purified GacB-GFP-fusions. The MALDI-MS analysis of the reaction products from GacB-D160N-GFP and GacB-Y182F-GFP revealed that both mutants lacked an enzymatic activity in vitro (FIGS. 4G and H). These results support the hypothesis that the residues D160 and Y182 play a role in substrate binding or catalysis.

Finally, we created three truncated versions of GacB at the N-terminal end as an attempt to determine whether the enzyme remains active in the absence of the residues predicted to be associated with the membrane. Our results showed that truncations of the first 22 (GacB₂₃₋₃₈₅), 75 (GacB₇₆₋₃₈₅) and 118 residues (GacB₁₁₉₋₃₈₅) led to inactivation of the enzyme when assessed through the complementation assay. Their inability to complement ΔgacB suggest that the N-terminal domain is required for activity and supports the hypothesis that GacB is a membrane-associated rhamnosyltransferase.

GacB is a Retaining β-1,4-Rhamnosyl-Transferase

The current gene annotation suggests that GacB is an inverting α-1,2 rhamnosyltransferase (1, 8). This annotation is incompatible with the acceptor sugar GlcNAc since its carbon at position C2 is already decorated with the N-acetyl group. Therefore, GacB can only transfer the rhamnose onto the available hydroxyl groups on C3, C4 or C6. In addition, the GAC backbone is composed of repeating units of rhamnose connected via an α-1,3-1,2 linkage (9, 12) suggesting that GacB would be the only rhamnosyltransferase of this pathway using a retaining mechanism of action. According to the CAZy database, the GacB sequence is classified as a GT-4 family member, which are classified as retaining GTs (27). If that classification is correct for GacB, the stereochemical configuration at the anomeric centre of the sugar donor, TDP-β-L-rhamnose, should be retained in the final product.

In order to elucidate whether GacB is an inverting or a retaining rhamnosyltransferase, we conducted nuclear magnetic resonance (NMR) spectroscopy on the purified reaction products 1 and 2. ¹H NMR spectra were collected at 800 MHz to both establish the structural integrity of acceptors 1 and 2 (FIG. 7A) and to determine the chemical structure of their products after the enzymatic reaction (Product 1 and 2). The NMR parameters were determined through one and two-dimensional (1D and 2D) and 2D total correlation spectroscopy (TOCSY) experiments (FIG. 7B); their chemical shifts are summarised in Table 2. For both acceptors, the anomeric proton of α-D-GlcNAc appeared as a doublet of doublets with 3J(H1,H2)=3.4 Hz, and 3J(H1,P)=7.2 Hz. Proton H2 of α-D-GlcNAc was also split by a 3J(H2,P)=2.4 Hz coupling with P. A 2D 1H, 31P HMQC spectrum (data not shown) revealed a correlation of both of these H-1′ protons with P at −13.5 ppm. Another correlation appeared between the 31P at −10.6 ppm and protons of the adjacent CH₂ groups of the alkyl chain, confirming the integrity of the acceptor substrate. For acceptor 2 a typical pattern of signals of a monosubstituted benzene with integral intensities of 2:2:1 was observed.

The addition of rhamnose to both acceptor substrates was accompanied by the appearance of a characteristic signal in the anomeric region of the spectrum (4.88 ppm, H1) next to the water signal. The anomeric configuration of this monosaccharide was established in several ways. The measured ³J(H1,H2) coupling constant of 1.0 Hz indicated a β-L configuration (1.1 and 1.8 Hz reported) for β-L and α-L-Rha, respectively). A rotating-frame nuclear Overhauser effect (ROESY) spectrum (FIG. 4B) showed spatial proximity of H1 of rhamnose with four other protons. Among these were H2, H3 and H5 protons of rhamnose, the latter two confirming a 1,3 diaxial arrangement between H1, H3 and H5 that is indicative of a β-L Rha configuration. Finally, a comparison of ¹H chemical shifts of rhamnose with those of α-L and β-L-rhamnopyranose (FIG. 7C) showed a good agreement with those of β-L-rhamnose (75), thus confirming configuration of this ring. The forth ROESY cross peak of H1 of rhamnose was with H4 of GlcNAc, revealing the presence of a (1-4) linkage between the two monosaccharides. This observation was further supported by a comparison of GlcNAc 1H chemical shifts of acceptor substrates and products. Here, an increased chemical shift (+0.21 ppm) was observed for H4 upon glycosylation, while the average of the absolute values of the differences between the chemical shifts of the other corresponding protons of GlcNAc was 0.03 ppm. As expected, the signals of the alkyl and aryl sidechains practically did not change in the respective acceptor-product pairs.

In conclusion, ¹H NMR spectroscopy revealed the formation of a R-L-Rha (1-4) D-GlcNAc moiety and the integrity of the product.

Group a, B, C and G Streptococcus Share a Common RhaPS Initiation Step

In addition to S. mutans SccB, GacB homologs with a high degree of sequence identity are found in other streptococcal species of clinical importance, such as the Streptococcus species from Group B (GBS), Group C (GCS) and Group G (GGS). All homologous enzymes are situated in the corresponding gene clusters encoding the biosynthesis of their Lancefield antigens, i.e., the Group B, C and G carbohydrate (15). The homologous gene products share 67%, 89% and 89% amino acid identity to GacB, respectively (Table 2, FIG. 8). With varying degrees of evidence depending on the species, there is a general understanding of the chemical structure of the RhaPS of these streptococci (9). The currently accepted structures for GAC, GBC, GCC, GGC and SCC are summarised in FIG. 8. Remarkably, none of the investigations that led to the understanding of the surface carbohydrate structures includes data describing the mechanism of action of the enzymes involved in the priming step of each RhaPS biosynthesis.

Based on the high-sequence identity to GacB, we hypothesised that the carbohydrate biosynthesis of the Group A, Group B, Group C and Group G Streptococcus possess a conserved initiation step, in which the first rhamnose residue is transferred onto the lipid-linked acceptor forming Rha-β-1,4-GlcNAc-PP-Und. We tested the ability of the homologs from GBS, GCS and GGS (GbsB, GcsB and GgsB, respectively) to functionally substitute GacB in the production of the RhaPS chain (FIG. 9). Our results show that all homologous proteins were able to restore the RhaPS backbone when their genes were co-expressed with the ΔgacB expression plasmid, suggesting these enzymes can perform the same enzymatic reaction.

We showed that GacB requires GlcNAc-PP-Und as acceptor, but it is possible that the enzymes from GBS, GCS and GGS use a different lipid-linked acceptor substrate, such as Glc-PP-Und. Thus, to determine whether the GacB homologs require GlcNAc-PP-Und as lipid acceptor, we conducted the complementation assay using E. coli ΔwecA cells, which lack GlcNAc-PP-Und (23). As a positive control we identified S. pneumoniae WchF, a Glc-1,4-β-rhamnosyltransferase that uses exclusively Glc-PP-Und as substrate (28). As expected, GacB was unable to restore the RhaPS chain when co-transformed with the ΔgacB vector in the absence of the GlcNAc-PP-Und (FIG. 9A, lane 2). The GacB homologs from GBS, GCS and GGS also failed to produce the RhaPS backbone (FIG. 9A, lane 4-6), but could replace GacB function in the ArfaS strain (FIG. 9B). Only WchF, which uses a Glc-PP-Und acceptor for the transfer of a rhamnose residue, restored the RhaPS biosynthesis in the absence of GlcNAc-PP-Und (FIG. 9A, lane 3). Combined with the data from our in vitro enzymatic reactions, these results suggest that the GacB homologues from GBS, GCS and GGS are also GlcNAc-1,4-β-rhamnosyltransferases that require GlcNAc-PP-Und as membrane-bound acceptor.

Most Streptococcal Pathogens are Predicted to have a GlcNAc-1,4-β-Rhamnosyl-Transferase

S. pneumoniae wchF encodes a Glc-β-1,4-rhamnosyltransferase that requires Glc-PP-Und as acceptor (28). It shares 51% amino acid identity to GacB, compared to 67-89% for the homologous enzymes from GBS, GCS, GGS and S. mutans. Towards a better understanding of the conservation of GacB in the Streptococcus genus, we extended our bioinformatics analysis to search for other strains that harbour GacB homologous genes. We found 48 human/veterinary pathogenic Streptococcus species with a single GacB homolog, sharing 50 to 94% sequence identity (Table 2, FIG. 10). Five of our 48 identified species showed a percentage identity equal or lower than 51% (S. mitis, S. pneumoniae, S. oralis subsp. tigurinus, S. peroris and S. pseudopneumoniae), while all other encoded proteins presented more than 65% homology to GacB. For simplicity, we will refer to the five Streptococcus strains with low amino acid identity as ‘low identity’ subgroup, and the rest of the species as the ‘high identity’ subgroup.

The sequence analysis paired with the complementation assay led us to hypothesise that all GacB homologs encompassed in the ‘high identity’ subgroup possess GlcNAc-β-1,4-rhamnosyltransferase activity. In contrast, the ‘low identity’ subgroup contains S. pneumoniae WchF, a known Glc-1,4-β-rhamnosyltransferase (28). All five members of the ‘low identity subgroup’ exhibit very high sequence identity (>90%) when compared to WchF.

GacO from S. pyogenes, the WecA homolog, was shown to be responsible for the biosynthesis of the GlcNAc-PP-Und (8,9), the substrate for GacB. We therefore hypothesised that the ‘low’ and ‘high identity’ subgroups utilise different substrates, and therefore investigated whether an equivalent discrepancy should be observed when comparing the sequence identity of the GacO homologs. Within the 48 pathogenic streptococci genomes (Table 2, FIG. 10), we found that all strains from the ‘high identity’ subgroup share a gacO homologue with 63-92% sequence identity. Importantly, any genome from the ‘low identity’ subgroup contains a gene product with equal or less than 30% sequence identity to GacO. This subgroup present gene products that have high homology to S. pneumoniae Cps2E, which transfers Glc-1-P to P-Und, to generate Glc-PP-Und (28). S. mitis, S. oralis subsp. tigurinus, S. peroris and S. pseudopneumoniae homologues share 98% sequence identity to Cps2E.

The degree of phylogenetic conservation of GacB in the Streptococcus genus highlights the importance of this gene, for survival and pathogenesis of streptococcal pathogens. Overall, these results lead us to propose that those streptococcal species that have GacB homologs with a high degree of identity (>65%) are GlcNAc-β-1,4-rhamnosyltransferases that catalyse the first committed step in the biosynthesis of their surface RhaPS by transferring rhamnose from TDP-β-L-rhamnose to the membrane-bound GlcNAc-PP-Und. In contrast, we postulate that the species within the ‘low identity’ subgroup, in accordance with the function of S. pneumoniae serotype 2 WchF, contains a rhamnosyltransferase that acts on lipid-linked Glc-PP-Und.

TABLE 2
Sequence conservation in % for GacB and GacO homologous
enzymes from 48 species of the Streptococcus genus.

| | % Identity | | | |
|---|---|---|---|---|
| Species | GacB | N-terminus | C-Terminus | GacO |
| S. pyogenes | 100 | 100 | 100 | 100 |
| S. canis | 94 | 94 | 92 | 92 |
| S. dysgalactiae subsp. | 89 | 92 | 86 | 90 |
| equisimilis | | | | |
| S. phocae | 79 | 83 | 75 | 85 |
| S. equi subsp. zooepidermicus | 77 | 78 | 75 | 86 |
| S. equi subsp. equi | 76 | 74 | 73 | 86 |
| S. ictaluri | 75 | 79 | 72 | 80 |
| S. bovimastitidis | 73 | 77 | 69 | 80 |
| S. iniae | 73 | 74 | 72 | 81 |
| S. hongkongensis | 72 | 77 | 68 | 80 |
| S. panaeicida | 72 | 78 | 68 | 81 |
| S. uberis | 72 | 76 | 67 | 81 |
| S. porcinus | 71 | 75 | 68 | 80 |
| S. henryi | 70 | 70 | 70 | 75 |
| S. orisasini | 70 | 70 | 69 | 75 |
| S. orisratti | 70 | 70 | 69 | 73 |
| S. parasanguinis | 69 | 71 | 66 | 65 |
| S. ratti | 69 | 70 | 53 | 76 |
| S. vestibularis | 69 | 68 | 68 | 70 |
| S. australis | 68 | 71 | 66 | 63 |
| S. equinus | 68 | 71 | 65 | 78 |
| S. porci | 68 | 69 | 67 | 71 |
| S. sanguinis | 68 | 71 | 65 | 67 |
| S. sinensis | 68 | 69 | 66 | 66 |
| S. sobrinus | 68 | 69 | 64 | 72 |
| S. thoraltensis | 68 | 70 | 66 | 71 |
| S. anginosus | 67 | 69 | 65 | 66 |
| S. caballi | 67 | 66 | 67 | 74 |
| S. downei | 67 | 70 | 65 | 72 |
| S. gordonii | 67 | 68 | 66 | 63 |
| S. intermedius | 67 | 70 | 64 | 67 |
| S. constellatus | 66 | 69 | 64 | 66 |
| S. gallolyticus | 66 | 68 | 66 | 78 |
| S. hyovaginalis | 66 | 69 | 64 | 71 |
| S. mutans | 66 | 51 | 61 | 75 |
| S. salivarius | 66 | 59 | 63 | 71 |
| S. urinalis | 66 | 69 | 64 | 74 |
| S. agalactiae | 65 | 66 | 64 | 73 |
| S. entericus | 65 | 63 | 67 | 66 |
| S. infantarius | 65 | 68 | 62 | 78 |
| S. plurextorum | 65 | 69 | 62 | 68 |
| S. suis | 65 | 67 | 62 | 68 |
| S. lutetiensis | 64 | 68 | 61 | 78 |
| S. oralis subsp. tigurimus | 51 | 46 | 52 | 28 |
| S. mitis | 50 | 45 | 51 | 29 |
| S. peroris | 50 | 46 | 50 | 28 |
| S. pneumoniae | 50 | 45 | 51 | 30 |
| S. pseudopneumoniae | 50 | 44 | 68 | 29 |

GacB's N-Terminal Domain Encodes Specificity for the GlcNAc Acceptor

We performed a multiple sequence alignment of the GacB homologs from all 48 streptococcal pathogens to identify the most variable and conserved regions in the protein sequence. We observed a higher discrepancy between the ‘high identity’ and the ‘low identity’ subgroups in their N-terminal domains (Table 2). More precisely, a low sequence conservation region is identifiable between the GacB amino acid residues 40 and 80, suggesting that this section of the domain is either involved in the GlcNAc acceptor sugar recognition or in essential protein-protein interactions.

We knew from our previous experiment that GacB cannot initiate the RhaPS biosynthesis on a wecA deletion background (FIG. 9A, lane 2). Based on this information and in order to identify residues involved in sugar acceptor recognition, we introduced mutations in the GacB amino acid sequence. The goal was to salvage the RhaPS initiation step in a wecA-deficient E. coli strain in which GacB mutants recognise a lipid-linked sugar acceptor other that GlcNAc-PP-Und.

Therefore, we investigated a structural model based on the GacB homolog from Bacillus anthracis, BaBshA (PDB entry 3mbo), which suggested that residues L128, R131, GNT100 may potentially be involved in sugar acceptor recognition. We mutated these residues to mimic those found in WchF. Complementation assays using GacB L128H_R131L, failed to complement ΔgacB in a ΔwecA background (FIG. 11, lane 2). Following a sequential approach, we modified the GacB primary sequence by introducing additional amino acid substitutions that corresponded to those found in WchF: L128H_R131L_GNT100ARC and L128H_R131L_GNT100ARC_A105P. None of these mutants recognised glucose to initiate the rhamnose chain, and thus, did not restore GacB's activity. Finally, we replaced the first 178 residues of GacB with the corresponding WchF amino acids (1-186). When expressed in a wecA deletion background, this WchF-GacB chimera was able to synthesise the RhaPS backbone on the exclusive acceptor substrate Glc-PP-Und (FIG. 11, lane 5).

Discussion

This work sheds light on the first committed step of the GAC biosynthesis and provides insight into the function of GacB, the first metal-independent, retaining and non-processive α-D-GlcNAc β-1,4-L-rhamnosyltransferase reported. This insight is depicted schematically in FIG. 12, which shows the elucidated structure of GAC as well as the endogenous S. mutans enzymes involved in the synthesis of each section. Other enzymes from Gram-negative and Gram-positive bacteria that are involved in polysaccharide biosynthesis use lipid-linked GlcNAc as acceptor and either dTDP-L- or GDP-D-rhamnose sugar nucleotides, however, their reaction results in an α-1,3 or α-1,4 glycosidic bond (29-31). Also, the fact that the GAC backbone is composed of repeating units of rhamnose connected via an α-1,3-1,2 linkage (9, 13) suggest that GacB is the only rhamnosyltransferase of this pathway using a retaining mechanism of action.

We have also shown that streptococcal RhaPS can be synthesized in a recombinant expression system, namely E. coli, onto a different acceptor, Und-PP-Glu using the enzyme WchF. This is depicted schematically in FIG. 13. Specifically, FIG. 13 demonstrates how the enzyme WchF can be used to transfer a rhamnose moiety to a glucose monosaccharide to form a disaccharide, the disaccharide having the glucose at the reducing end and the rhamnose moiety at the non-reducing end. The enzyme WchF facilitates the formation of a β-1,4 glycosidic bond between the two monosaccharides. A rhamnose polysaccharide is then generated by extended from the rhamnose moiety at the non-reducing end of the disaccharide using the bacterial enzyme GacC or its enzymatically active homologue GbcC. WchF is derived from S. pneumoniae, this is heterologous to the bacteria (S. mutans and S. agalactiae) from which GacC or GbcC are derived. In this particular embodiment, the method was carried out in E. coli, which is also a different species to the bacteria from which WchF, GacC and GbcC are derived.

This results in the formation of a synthetic streptococcal polysaccharide having a non-reducing end comprising a linear chain of rhamnose moieties and a reducing end comprising a glucose monosaccharide, the polysaccharide comprising a β-1,4 bond between the glucose and the linear chain of rhamnose moieties. As the skilled person will appreciate, this differs from the naturally occurring GAC (which is shown in FIG. 12) due to the monosaccharide at the reducing end being glucose rather than GlcNAc.

### EXAMPLE 2

To further illustrate the invention, this Example is directed to further exemplary methods of synthesis and the rhamnose polysaccharide of the invention.

FIG. 14 is another exemplary embodiment of the invention. FIG. 14 shows how the enzyme WbbL, which is derived from E. coli, can be used to transfer a rhamose moiety to a GlcNAc monosaccharide. This forms a disaccharide having the GlcNAc at its reducing end and the rhamnose moiety at the non-reducing end with an α-1,3 glycosidic bond between the rhamnose moiety and the GlcNAc. The rhamnose polysaccharide is then generated by extension from the rhamnose moiety at the reducing end of the disaccharide using the bacterial enzyme GacC or its enzymatically active homologue GbcC. Since WbbL is derived from E. coli, it is derived from a bacterial species heterologous to the bacterial species from which GacC and GbcC are derived.

In this particular example, the method is performed in E. coli, although other bacteria can be envisaged for this purpose. Thus, in this particular embodiment, WbbL can be endogenous to the E. coli or it can be overexpressed in the E. coli.

This method, as FIG. 14 shows, results in the generation of a synthetic streptococcal polysaccharide having a non-reducing end comprising a linear chain of rhamnose moieties and a reducing end comprising a GlcNAc monosaccharide, the polysaccharide comprising a α-1,3 bond between the GlcNAc and the linear chain of rhamnose moieties. This differs from the endogenous GAC (as shown in FIG. 12), as GAC contains a β-1,4 bond between the GlcNAc and the linear chain of rhamnoses. Any other enzyme which is a hexose-α-1,3-rhamnosyltransferase could be used instead of WbbL, as shown schematically in FIG. 15. FIG. 15 differs from FIG. 14 in that the monosaccharide is a glucose rather than a GlcNAc. Thus, the product of FIG. 14 is a synthetic Streptococcal polysaccharide having a non-reducing end comprising a linear chain of rhamnose moieties and a reducing end comprising a glucose monosaccharide, the polysaccharide comprising a α-1,3 bond between the glucose and the linear chain of rhamnose moieties. This differs from the endogenous GAC (shown in FIG. 12) with the inclusion of the glucose and the α-1,3 bond.

Other methods of synthesis are within the scope of the present invention. FIG. 16 shows such an exemplary method. In this method, a diNAcBac-α-1,3-rhamnosyltransferase is used to transfer a rhamnose moiety to a diNAcBac monosaccharide. Thus, a disaccharide is formed having the diNAcBac at its reducing end and the rhamnose moiety at the non-reducing end. The two monosaccharides are linked with an α-1,3 glycosidic bond. The rhamnose polysaccharide is then generated by extended from the rhamnose moiety at the non-reducing end of the disaccharide using the bacterial enzyme GacC or its enzymatically active homologue GbcC. The diNAcBac-α-1,3-rhamnosyltransferase is derived from a bacterial species different to the bacterial species from which GacC or its enzymatically active homologue GbcC is derived.

The method of FIG. 16 leads to the generation of a synthetic streptococcal polysaccharide having a non-reducing end comprising a linear chain of rhamnose moieties and a reducing end comprising diNAcBac monosaccharide, the polysaccharide comprising a α-1,3 bond between the diNAcBac and the linear chain of rhamnose moieties. This differs from the endogenous GAC (as shown in FIG. 12), as GAC contains a β-1,4 bond between a GlcNAc and the linear chain of rhamnoses.

FIG. 17 demonstrates another exemplary method and product. In this method, a disaccharide, trisaccharide or tetrasaccharide can be formed before extending from the rhamnose moiety. For the disaccharide, the galactose-α-1,2-rhamnosyltransferase WbbR is used to transfer a rhamnose moiety to a galactose monosaccharide. This forms a disaccharide having the galactose at its reducing end and the rhamnose moiety at its non-reducing end. The rhamnose polysaccharide is then generated by extending from this rhamnose moiety to form a linear chain of rhamnose moieties. In this example, extension is using the enzymes GacC, GacG or GbcC (see penultimate schematic of FIG. 17 and top schematic). WbbR is derived from Shigella, which is a different bacterial species to the Streptococcus from which GacC, GacG or GbcC are each derived. This method leads to the production of a synthetic streptococcal polysaccharide having a non-reducing end comprising a linear chain of rhamnose moieties and a reducing end comprising a galactose monosaccharide, the polysaccharide comprising a α-1,2 bond between the diNAcBac and the linear chain of rhamnose moieties.

An alternative embodiment, as also depicted by the top and penultimate schematics of FIG. 17, is the formation of a trisaccharide before extending from the rhamnose moiety. For the trisaccharide, the enzyme WbbP is used to transfer a galactose monosaccharide to a GlcNAc, thus forming an α-1,3 glycosidic bond between the two monosaccharides. The enzyme WbbR is then used as described above for the disaccharide such that a rhamnose moiety is transferred to the galactose. After this extension can occur as detailed for the disaccharide above.

To the left of FIG. 17 is a spot blot (positive antibody blot). Each blot represents a sample from one experiment; each row represents a triplicate of the same conditions. For each experiment, the sample from the reaction was added as a spot, and an anti-GAC antibody used to determine if the reaction was successful in the formation of the rhamnose polysaccharide. The middle row shows triplicates of samples obtained from reactions where the enzyme WbbP is used to transfer a galactose monosaccharide to a GlcNAc, followed by the enzyme WbbR then GacG. The dot plot to the left confirms that this reaction is capable of producing the rhamnose polysaccharide of the invention.

WbbP can alternatively be used to form a disaccharide (i.e., a galactose monosaccharide at its non-reducing end linked by an α-1,3 glycosidic bond to a GlcNAc at its reducing end, following which the rhamnose polysaccharide is generated by extended from the rhamnose moiety at the non-reducing end of the disaccharide (see bottom schematic of FIG. 17). The dot plot row to the left of this schematic confirms that this reaction is also capable of producing the rhamnose polysaccharide of the invention.

Optionally, one or two additional rhamnose moieties can be transferred to the rhamnose moiety linked to the galactose to form a tetra or pentasaccharide, prior to the step of extension as detailed above. The one or two additional rhamnose moieties can be transferred using the enzyme WbbQ, followed by further extension using GacC using GbcC, as shown in the third schematic of FIG. 17. The dot plot row to the left of this Figure confirms that a reaction containing WbbP, WbbR, WbbQ and GacC was successful in generating a rhamnose polysaccharide according to the present invention.

For the tri, tetra or pentasaccharide methods, these methods result in the generation of a synthetic Streptococcal polysaccharide having a reducing end comprising a linear chain of rhamnose moieties and a non-reducing end comprising a GlcNac and a galactose, the polysaccharide comprising a α-1,2 bond between the linear chain of rhamnose moieties and the galactose and a α-1,3 bond between the galactose and the GlcNAc.

In embodiments wherein a rhamnose moiety is transferred to a disaccharide or trisaccharide, it is envisaged that any combination of hexoses may be used to form the di or trisaccharide using alpha or beta bonds as described herein. This is depicted in FIG. 18. Likewise, for the extension of the rhamnose polysaccharide from the rhamnose moiety, it is envisaged that any enzymatically active homologue of GacC, GacG, or a fragment or variant thereof, could be used, provided that α-1,2 and/or α-1,3 glycosidic bonds are formed between each pair of rhamnose moieties.

FIG. 19 confirms that WbbL can be used instead of GacB or SccB in a method of the invention to produce the rhamnose polysaccharide. The figure shows an anti-GAC Western blot of total E. coli lysate from cells expressing the gene cluster RmlD-SccC-SccD-SccE-SccF-SccG (deltaSccB) and GacA-GacC-GacD-GacE-GacF-GacG (deltaGacB) complemented with empty plasmid controls or WbbL. The first column is a ladder. The second column confirms that GAC was not produced in E. coli cells having a RgpA deletion, while the third column confirms that the expression of WbbL alone in RgpA deficient cells did not restore GAC synthesis. The third column shows the lysate from E. coli cells having a RgpA deletion but also expressing the gene cluster GacA-GacC-GacD-GacE-GacF-GacG (deltaGacB). No GAC was found in these cells. However, the fourth column shows that when WbbL is expressed in the cells of the third column, GAC is produced. The same result is observed when rgpA deficient cells express the gene cluster RmlD-SccC-SccD-SccE-SccF-SccG (deltaSccB) together with WbbL (see duplicates of last two columns). This data confirms that WbbL can be used with heterologous enzymes from other species to produce a rhamnose polysaccharide according to the present invention.

FIG. 20 confirms that GacC introduces up to five Rhamnose sugars onto the product generated from GacB. FIG. 20 shows radiolabelling of lipid-linked oligosaccharides (LLOS) in vivo (E. coli). Film exposure of a TLC plate with radiolabelled LLOS from E. coli CS2775 bearing gacB (lane 1) or gacBC (lane 2).

Homologues to GacC can function in a similar manner. FIG. 21 shows results similar to that shown in FIG. 20, but using GbcC, GccC and GgcC, from homologous enzymes from Group B, C and G Streptococci. FIG. 21 shows a film exposure of a TLC plate with radiolabelled LLOS from E. coli CS2775 bearing gacB and gacC (lane 1), gacB alone (lane 2), gacB and gbcC (lane 3), gacB and gccC (lane 4), gacB and ggcC (lane 5). GacC, GbcC, GccC, GgcC are homologous enzymes from Group A, B, C and G Streptococci and the figure shows that all transfer 3-5 rhamnose sugars onto the product of GacB.

Similarly, the inventor has shown that the GacC enzyme function is conserved amongst Streptococci and is able to complement SccC enzyme of E. coli. FIG. 22 shows:
A) Gene complementation strategy. sccC gene replaced with homologous genes gacC, gbcC, gccC, ggcC.
B) Immunoblots of whole-cell lysates for the bacterial complementation assay probed with anti-Group A antibody.

Complementation study confirms that GacC enzyme function is conserved amongst Streptococci from Group B, C, G and S. mutans.

Phylogenetic analysis of GacO, GacB and GacC enzymes show the high degree of similarity and hence function is conserved in Streptococci-Pathogenic strains are all expected to produce RhaPS with identical adapter/stem and as such, all are suitable for use in accordance with the present invention.

FIG. 23 shows A) Phylogenetic tree based on GacB ortholog protein sequences identified from forty-eight pathogenic streptococci. An asterisk after the species name indicates that the ortholog sequence was not retrieved from a whole sequenced genome. Sequences were aligned using the default neighbour-joining clustering method of ClustalOmega and then plotted using iTOL online tool. B) The bar charts indicate the degree of homology in percentage to S. pyogenes GacO (red), GacB (blue) or GacC (green). The figures next to GacO, GacB and GacC labels represent the step catalysed by S. pyogenes. The figures in the indentation at the centre of the figure is based on our current knowledge of the role of S. pneumoniae Cps2E, Cps2T (WchF) and Cps2F (James 2013).

FIG. 24 shows that GacC rhamnosylates synthetic LLO substrate (GacB product) in vitro. A) HPLC analysis showing that GacC extends a chemoenzymatic lipid-linked disaccharide generated using GacB with 3 additional rhamnose residues. The chemical linkage was subsequently analysed by NMR. B) Chemical drawing of GacB/C reactions with in vitro acceptor substrate

Further studies, not all data shown, by the inventors using NMR and mass spectrometry techniques confirm that GacC can add up to 4 rhamnose sugars and that GacC is an inverting alpha-1,3 rhamnosyltransferase. FIG. 25 shows full assignment of protons and carbon sugar signals. ¹H assignments were based on the analysis of several F1-band-selective 2D TOCSY spectra. ³C signals were assigned using 2D ¹H, ¹³C HSQC. Linkages were assigned using a 2D NOESY experiment. Chemical shifts for each of the sugar residues agrees well with published data for 1H and 13C signals for glycopyranoses.

The inventor has further shown that the rhamnose polysaccharide in accordance with the present invention may be generated using different enzyme combinations. FIG. 26 shows that the rhamnose polysaccharide according to the present invention may be generated using enzymes from Shigella dysenteriae in combination with E. coli and Shigella dysenteriae in combination with Streptococcus mutans. FIG. 26 shows a whole cell Western blot using anti-Group A Carbohydrate antibody. Total E. coli cell lysates were separated over SDS-PAGE. NewRhaPS are build by Shigella dysenteriae gene products combined with S. mutans/Group A Streptococcus gene products. RmlD_GacD_E_F_G plus WbbP_Q_R are sufficient to build NewRhaPS. NewRhaPS can also be build with RmlD_SccC_D_E_F_G plus WbbP_Q_R.

Based on the above evidence, it is expected that Shigella spp. can be further used in order to provide the adaptor/stem and GAC repeat units, as shown schematically in FIG. 27. In a native system, GacB and GacC enzymes install the adaptor/stem region (red box) before GacG installs the immunogenic repeat unit. The figure shows as an example 3 alpha1,3-rhamnose sugars installed by GacC.

Replacement of the GacB/C enzymes (replacement of the GlcNAc-beta1,4-rhamnose-alpha1,3-rhamnose adaptor/stem) to generate NewRhaPS, provides an alternative to maintain the immunogenic repeat unit (proposed to be introduced by GacG enzyme activity). Replacing the adaptor region (green box) with a O-Otase compatible polysaccharide/oligosaccharide is sufficient to build the immunogenic polysaccharide (alpha1,2-alpha1,3 rhamnose).

As described herein, the rhamnose polysaccharides of the present invention may be conjugated with a suitable protein and presented on the surface of a bacterium. FIG. 28 shows that rhamnose polysaccharides prepared in accordance with the present invention are suitable substrates for use in an E. coli glycoconjugation system. A periplasmic expressin test system was set up in accordance with the procedure described by Reglinski et al., npj Vaccines (2108)3:53._{[HD(1]}FIG. 28 shows that NewRhaPS are compatible substrate for O-Otase (PglB)/for Protein Glycan Coupling Technology (PGCT) Periplasmic expression of test protein NanA (in accordance with Reglinski)+/− active/inactive NewRhaPS system (1-8).

Lanes 5 and 7 show that two different expression conditions for NewRhaPS system are positive for NanA-NewRhaPS glycosylation.

Lane 9: GAC chemically extracted from S. pyogenes (positive control for GAC antibody).

This description should not be construed as limiting and it will be appreciated that other variants and embodiments thereof fall within the scope of the present invention.

### REFERENCES

1. van Sorge, N. M., Cole, J. N., Kuipers, K., Henningham, A., Aziz, R. K., Kasirer-Friede, A., Lin, L., Berends, E. T. M., Davies, M. R., Dougan, G., Zhang, F., Dahesh, S., Shaw, L., Gin, J., Cunningham, M., Merriman, J. A., HQtter, J., Lepenies, B., Rooijakkers, S. H. M., Malley, R., Walker, M. J., Shattil, S. J., Schlievert, P. M., Choudhury, B., and Nizet, V. (2014) The Classical Lancefield Antigen of Group A Streptococcus Is a Virulence Determinant with Implications for Vaccine Design. Cell Host Microbe. 15, 729-740
2. Kristian, S. A., Datta, V., Weidenmaier, C., Kansal, R., Fedtke, I., Peschel, A., Gallo, R. L., and Nizet, V. (2005) D-alanylation of teichoic acids promotes group a streptococcus antimicrobial peptide resistance, neutrophil survival, and epithelial cell invasion. J. Bacteriol. 187, 6719-6725
3. Henningham, A., Davies, M. R., Uchiyama, S., Sorge, N. M. van, Lund, S., Chen, K. T., Walker, M. J., Cole, J. N., and Nizet, V. (2018) Virulence Role of the GlcNAc Side Chain of the Lancefield Cell Wall Carbohydrate Antigen in Non-M1-Serotype Group A Streptococcus. mBio. 9, e02294-17
4. Le Breton, Y., Belew, A. T., Freiberg, J. A., Sundar, G. S., Islam, E., Lieberman, J., Shirtliff, M. E., Tettelin, H., El-Sayed, N. M., and McIver, K. S. (2017) Genome-wide discovery of novel M1T1 group A streptococcal determinants important for fitness and virulence during soft-tissue infection. PLoS Pathog. 13, e1006584
5. Shelburne, S. A., Keith, D., Horstmann, N., Sumby, P., Davenport, M. T., Graviss, E. A., Brennan, R. G., and Musser, J. M. (2008) A direct link between carbohydrate utilization and virulence in the major human pathogen group A Streptococcus. Proc. Natl. Acad. Sci. U.S.A. 105, 1698-1703
6. Lancefield, R. C. (1933) A Serological Differentiation of Human and Other Groups of Hemolytic Streptococci. J. Exp. Med. 57, 571-595
7. McCarty, M. (1958) Further studies on the chemical basis for serological specificity of group a streptococcal carbohydrate. J. Exp. Med. 108, 311-323
8. Rush, J. S., Edgar, R. J., Deng, P., Chen, J., Zhu, H., van Sorge, N. M., Morris, A. J., Korotkov, K. V., and Korotkova, N. (2017) The molecular mechanism of N-acetylglucosamine side-chain attachment to the Lancefield group A carbohydrate in Streptococcus pyogenes. J. Biol. Chem. 292, 19441-19457
9. Mistou, M.-Y., Sutcliffe, I. C., and Sorge, N. M. van (2016) Bacterial glycobiology: rhamnose-containing cell wall polysaccharides in Gram-positive bacteria. FEMS Microbiol. Rev. 40, 464-479
10. Coligan, J. E., Kindt, T. J., and Krause, R. M. (1978) Structure of the streptococcal groups A, A-variant and C carbohydrates. Immunochemistry. 15, 755-760
11. Krause, R. M., and McCarty, M. (1961) Studies on the Chemical Structure of the Streptococcal Cell Wall. J. Exp. Med. 114, 127-140
12. Edgar, R. J., Hensbergen, V. P. van, Ruda, A., Turner, A. G., Deng, P., Breton, Y. L., El-Sayed, N. M., Belew, A. T., McIver, K. S., McEwan, A. G., Morris, A. J., Lambeau, G., Walker, M. J., Rush, J. S., Korotkov, K. V., Widmalm, G., Sorge, N. M. van, and Korotkova, N. (2019) Discovery of glycerol phosphate modification on streptococcal rhamnose polysaccharides. Nat. Chem. Biol. 15, 463
13. H. Heymann, Zeleznick, L. D., Boltralik, J. J., Barkulis, S. S., and Smith, C. (1963) Biosynthesis of Streptococcal Cell Walls: A Rhamnose Polysaccharide. Science. 140, 400-401
14. Heymann, H., Manniello, J. M., and Barkulis, S. S. (1967) Structure of streptococcal cell walls. V. Phosphate esters in the walls of group A Streptococcus pyogenes. Biochem. Biophys. Res. Commun. 26, 486-491
15. van Hensbergen, V. P., Movert, E., de Maat, V., Luchtenborg, C., Le Breton, Y., Lambeau, G., Payre, C., Henningham, A., Nizet, V., van Strijp, J. A. G., BrQgger, B., Carlsson, F., McIver, K. S., and van Sorge, N. M. (2018) Streptococcal Lancefield polysaccharides are critical cell wall determinants for human Group IIA secreted phospholipase A2 to exert its bactericidal effects. PLoS Pathog. 14, e1007348
16. Sewell, E. W. C., and Brown, E. D. (2014) Taking aim at wall teichoic acid synthesis: new biology and new leads for antibiotics. J. Antibiot. (Tokyo). 67, 43-51
17. Huang, D. H., Rama Krishna, N., and Pritchard, D. G. (1986) Characterization of the group A streptococcal polysaccharide by two-dimensional 1H-nuclear-magnetic-resonance spectroscopy. Carbohydr. Res. 155, 193-199
18. van der Beek, S. L., Le Breton, Y., Ferenbach, A. T., Chapman, R. N., van Aalten, D. M. F., Navratilova, I., Boons, G.-J., McIver, K. S., van Sorge, N. M., and Dorfmueller, H. C. (2015) GacA is essential for Group A Streptococcus and defines a new class of monomeric dTDP-4-dehydrorhamnose reductases (RmlD). Mol. Microbiol. 98, 946-962
19. Le Breton, Y., Belew, A. T., Valdes, K. M., Islam, E., Curry, P., Tettelin, H., Shirtliff, M. E., El-Sayed, N. M., and McIver, K. S. (2015) Essential Genes in the Core Genome of the Human Pathogen Streptococcus pyogenes. Sci. Rep. 5, 9838
20. Shibata, Y., Yamashita, Y., Ozaki, K., Nakano, Y., and Koga, T. (2002) Expression and characterization of streptococcal rgp genes required for rhamnan synthesis in Escherichia coli. Infect. Immun. 70, 2891-2898
21. Bruyere, T., Wachsmann, D., Klein, J. P., Schöller, M., and Frank, R. M. (1987) Local response in rat to liposome-associated Streptococcus mutans polysaccharide-protein conjugate. Vaccine. 5, 39-42
22. Cartee, R. T., Forsee, W. T., Bender, M. H., Ambrose, K. D., and Yother, J. (2005) CpsE from type 2 Streptococcus pneumoniae catalyzes the reversible addition of glucose-1-phosphate to a polyprenyl phosphate acceptor, initiating type 2 capsule repeat unit formation. J. Bacteriol. 187, 7425-7433
23. Ozaki, K., Shibata, Y., Yamashita, Y., Nakano, Y., Tsuda, H., and Koga, T. (2002) A novel mechanism for glucose side-chain formation in rhamnose-glucose polysaccharide synthesis. FEBS Lett. 532, 159-163
24. Vetting, M. W., Frantom, P. A., and Blanchard, J. S. (2008) Structural and enzymatic analysis of MshA from Corynebacterium glutamicum: substrate-assisted catalysis. J. Biol. Chem. 283, 15834-15844
25. Jurtshuk, P. (1996) Bacterial Metabolism. in Medical Microbiology, 4th Ed. (Baron, S. ed), University of Texas Medical Branch at Galveston, Galveston (Tex.)
26. Parsonage, D., Newton, G. L., Holder, R. C., Wallace, B. D., Paige, C., Hamilton, C. J., Dos Santos, P. C., Redinbo, M. R., Reid, S. D., and Claiborne, A. (2010) Characterization of the N-acetyl-α-D-glucosaminyl I-malate synthase and deacetylase functions for bacillithiol biosynthesis in Bacillus anthracis. Biochemistry (Mosc.). 49, 8398-8414
27. Lombard, V., Golaconda Ramulu, H., Drula, E., Coutinho, P. M., and Henrissat, B. (2014) The carbohydrate-active enzymes database (CAZy) in 2013. Nucleic Acids Res. 42, D490-495
28. James, D. B. A., and Yother, J. (2012) Genetic and Biochemical Characterizations of Enzymes Involved in Streptococcus pneumoniae Serotype 2 Capsule Synthesis Demonstrate that Cps2T (WchF) Catalyzes the Committed Step by Addition of β1-4 Rhamnose, the Second Sugar Residue in the Repeat Unit. J. Bacteriol. 194, 6479-6489
29. Schägger, H. (2006) Tricine-SDS-PAGE. Nat. Protoc. 1, 16-22
30. Waldo, G. S., Standish, B. M., Berendzen, J., and Terwilliger, T. C. (1999) Rapid protein-folding assay using green fluorescent protein. Nat. Biotechnol. 17, 691-695
31. Druzhinina, T. N., Danilov, L. L., Torgov, V. I., Utkina, N. S., Balagurova, N. M., Veselovsky, V. V., and Chizhov, A. O. (2010) 11-Phenoxyundecyl phosphate as a 2-acetamido-2-deoxy-α-d-glucopyranosyl phosphate acceptor in O-antigen repeating unit assembly of Salmonella arizonae O:59. Carbohydr. Res. 345, 2636-2640
32. Robinson, P. T., Pham, T. N., and Uhrin, D. (2004) In phase selective excitation of overlapping multiplets by gradient-enhanced chemical shift selective filters. J. Magn. Reson. San Diego Calif. 1997. 170, 97-103
33. Rucker, F. J., and Osorio, D. (2008) The effects of longitudinal chromatic aberration and a shift in the peak of the middle-wavelength sensitive cone fundamental on cone contrast. Vision Res. 48, 1929-1939

SEQUENCES
GacC
SEQ ID NO: 1
MNINILLSTYNGERFLAEQIQSIQRQTVNDWTLLIRDDGSTDGTQDIIRTFVKEDKRIQW
INEGQTENLGVIKNFYTLLKHQKADVYFFSDQDDIWLDNKLEVTLLEAQKHEMTAPLLVYTD
LKVVTQHLAVCHDSMIKTQSGHANTSLLQELTENTVTGGTMMITHALAEEWTTCDGLLMHD
WYLALLASAIGKLVYLDIPTELYRQHDANVLGARTWSKRMKNWLTPHHLVNKYWWLITSSQ
KQAQLLLDLPLKPNDHELVTAYVSLLDMPFTKRLATLKRYGFRKNRIFHTFIFRSLVVTLFGY
RRK
GacG
SEQ ID NO: 2
MNRILLYVHFNKYNKISAHVYYQLEQMRSLFSKIVFISNSKVSHEDLKRLKNHCLIDEFL
QRKNKGFDFSAWHDGLIIMGFDKLEEFDSLTIMNDTCFGPIWEMAPYFENFEEKETVDFWG
ITNNRGTKAFKEHVQSYFMTFKNQVIQNKVFQQFWQSIIEYENVQEVIQHYETQLTSILLNEG
FSYQTVFDTRKAESSFMPHPDFSYYNPTAILKHHVPFIKVKAIDANQHIAPYLLNLIRETTNYP
IDLIVSHMSQISLPDTKYLLSQKYLNCQRLAKQTCQKVAVHLHVFYVDLLDEFLTAFENWNF
HYDLFITTDSDIKRKEIKEILQRKGKTADIRVTGNRGRDIYPMLLLKDKLSQYDYIGHFHTKKS
KEADFWAGESWRKELIDMLVKPADSILSAFETDDIGIIIADIPSFFRFNKIVNAWNEHLIAQEM
MSLWRKMDVKKQIDFQAMDTFVMSYGTFVWFKYDALKSLFDLELTQNDIPSEPLPQNSILH
AIERLLVYIAWGDSYDFRIVKNPYELTPFIDNKLLNLREDEGAHTYVNFNQMGGIKGALKYIIV
GPAKAMKYIFLRLMEKLK
RfbG
SEQ ID NO: 3
MHSSDQKRVAVLMATYNGECWIEEQLKSIIEQKDVDISIFISDDLSTDNTLNICEEFQLS
YPSIINILPSVNKFGGAGKNFYRLIKDVDLENYDYICFSDQDDIWYKDKIKNAIDCLVFN
NANCYSSNVIAYYPSGRKNLVDKAQSQTQFDYFFEAAGPGCTYVIKKETLIEFKKFIINNKNA
AQDICLHDWFLYSFARTRNYSWYIDRKPTMLYRQHENNQVGANISFKAKYKRLGLVRNKW
YRKEVTKIANALADDSFVNNQLGKGYIGNLILALSFWKLRRKKADKIYILLMLILNIF
GbcC
SEQ ID NO: 4
MKVNILMATYNGEKFLAQQIESIQKQTFKEWNLLIRDDGSSDKTCDIIRNFTAKDSRIRF
INENEHHNLGVIKSFFTLVNYEVADFYFFSDQDDVWLPEKLSVSLEAAKHKASDVPLLVYTD
LKVVNQELNILQDSMIRAQSHHANTTLLPELTENTVTGGTMMINHALAEKWFTPNDILMHDW
FLALLAASLGEIIYLDLPTQLYRQHDNNVLGARTMDKRFKILREGPKSIFTRYWKLIHDSQKQ
ASLIVDKYGDIMTANDLELIKCFIKIDKQPFMTRLRWLWKYGYSKNQFKHQVVFKWLIATNYY
NKR
GccC
SEQ ID NO: 5
MNINILLSTYNGERFLAEQIQSIQKQTIKDWTLLIRDDGSTDRTPDIIREFVKQDQRIQW
INENQIENLGVIKNFYTLLKYQAADVYFFSDQDDIWLEDKLEVTLLEAQKHDLSKPLLVY
TDLKVVNQQLEITHASMIKTQSAHANTTLLQELTENTVTGGTMMINQALAKEWNTCEGLLM
HDWYLALVAAARGKLVCLDIPTELYRQHDANVLGARTWSKRMKHWLRPHQLIRKYWWLIT
SSQQQAQLLLDLPLQPKDRDMVEAYVSLLTMSLTKRLATLKTYGFRKNRAFHTLVFWSLVIT
LFGYRRK
GqcC
SEQ ID NO: 6
MNINILLSTYNGERFLAEQIQSIQKQTIKDWTLLIRDDGSTDRTPDIIREFVKQDQRIQW
INENQIENLGVIKNFYTLLKYQAADVYFFSDQDDIWLEDKLEVTLLEAQKHDLSKPLLVY
TDLKVVNQQLEITHASMIKTQSAHANTTLLQELTENTVTGGTMMINQALAKEWNTCEGLLM
HDWYLALVAAARGKLVYLDIPTELYRQHDANVLGARTWSKRMKHWLRPHQLIRKYWWLIT
SSQQQAQLLLDLPLQPKDRDMVEAYVSLLTMSLTKRLATLKTYGFRKNRAFHTLVFWSLVIT
LFGYRRK
SccC
SEQ ID NO: 7
MKVNILMSTYNGQEFIAQQIQSIQKQTFENWNLLIRDDGSSDGTPKIIADFAKSDARIRF
INADKRENFGVIKNFYTLLKYEKADYYFFSDQDDVWLPQKLELTLASVEKENNQIPLMVYTD
LTVVDRDLQVLHDSMIKTQSHHANTSLLEELTENTVTGGTMMVNHCLAKQWKQCYDDLIM
HDWYLALLAASLGKLIYLDETTELYRQHESNVLGARTWSKRLKNWLRPHRLVKKYWWLVT
SSQQQASHLLELDLPAANKAIIRAYVTLLDQSFLNRIKWLKQYGFAKNRAFHTFVFKTLIITKF
GYRRK
SucC
SEQ ID NO: 8
MKINILMSTYNGEKFLAEQIESIQKQTVTDWTLLIRDDGSSDRTPEIIQDFVAKDSRIHF
INADHRINFGVIKNFFTLLKYEEADYYFFSDQDDVWLPHKIETSLNKAKELEKNRPFLIY
TDLTIVNQSLETIHESMISFQSDHANTTLLEELTENTVTGGTALINHALAELWTDDKDLL
MHDWFLALLASAMGNLVYINEATELYRQHDRNVLGARTWSKRLKTWSKPHLMLNKYWWLI
QSSQQQAQKLLDLPLSSDKRKLVEHYVTLLEKPLMTRLRDLKKYGYKKNRAFHTFVFRMLII
TKIGYRRTVKNGIIQ
GccG
SEQ ID NO: 9
MNRVLLYVHFNKYNKVSKHIYYQLEKLRPLFTTVVFISNSKVEQKELENLQKQRLIDSFI
QRENKGFDFAAWHDGMMKIGFDDLTLCDSLTIMNDTCFGPLWGMAPYFEKFDNNQSVDF
WGLTNNRKTSSFKEHIQSYFITFKQHVIQSDAFLNFWKTIKEYDDVQEVIQKYETQVTTTLLE
AGFNYQTVFDTREADSSFMLHPDFSYYNPTAILQHRVPFIKVKAIDANQHITPYLLNMIEEET
TYPVDLIISHMSQVGLPDAKYLLARKYLPFESLVTQNVPRIAVHLHVFYVDLLNEFLEGFASW
EFQYDLYITTDTQEKKEAIEKLLVQSNRHAHLYVTGNVGRDVLPMLLLKDKLRDYDYIGHFH
TKKSKEADFWAGESWRKELINMLIKPANEIVRSFENNDIGIVIADIPSFFRFNKIVDAWNEHLI
APEMMRLWKEMGLKKEIDFQSMDTFVMSYGTFVWFKFDALKPLFDLDLTVDDIPKEPLPQN
SILHAIERLLVYIAWDRFYDFRIVKNPYNLSPFIDNKLLNLRESGGARTYVNFDHMGGIKGAL
KYIIIGPARAMKYIVKRVLKSKR
GccG Protein 1
SEQ ID NO: 10
MNRVLLYVHFNKYNKVSKHIYYQLEKLRPLFTTVVFISNSKVEQKELENLQKQRLIDSFIQRE
NKGFDFAAWHDGMMKIGFDDLTLCDSLTIMNDTCFGPLWGMAPYFEKFDNNQSVDFWGL
TNNRKTSSFKEHIQSYFITFKQHVIQSDAFLNFWKTIKEYDDVQEVIQKYETQVTTTLLEAGF
NYQTVFDTREADSSFMLHPDFSYYNPTAILQHRVPFIKVKAIDANQHITPYLLNMIEEETTYP
VDLIISHMSQVGLPDAKYLLARKYLPFESLVTQNVPRIAVHLHVFYVDLLNEFLEGFASWEFQ
YDLYITTDTQEKRKQLKNY
GccG Protein 2
SEQ ID NO: 11
MGVSVRPLYYNRYSRKKEAIEKLLVQSNRHAHLYVTGNVGRDVLPMLLLKDKLRDYDYIGH
FHTKKSKEADFWAGESWRKELINMLIKPANEIVRSFENNDIGIVIADIPSFFRFNKIVDAWNEH
LIAPEMMRLWKEMGLKKEIDFQSMDTFVMSYGTFVWFKFDALKPLFDLDLTVDDIPKEPLP
QNSILHAIERLLVYIAWDRFYDFRIVKNPYNLSPFIDNKLLNLRESGGARTYVNFDHMGGIKG
ALKYIIIGPARAMKYIVKRVLKSKR
GgcG Protein 1
SEQ ID NO: 12
MIGKIIRSYQDEGGRATLRKIRQRLQGGGHPQSAGKIDLNRIPIMPQLEDIAQADYINHP
YQRPAKLDKKQLNIAWVSPPVGKGGGGHTTISRFVKYLQSQGHHITFYIYHNNTIEQSAKEA
QEIFSKAYGIEVAVDDLKNFSNQDLVFATSWETAYAVFNLKSENLHKFYFVQDFEPIFYGVG
SRYKLAEATYKFGFYGITAGKWLTHKLKDYHMDADYFNFGADTDIYKPKAPLQKKKKIAFYA
RAHTERRGFELGVMALKIFKDKHPEYDIEFFGQDMSHYDIPFDFIDRGILNKEELAAIYHESV
ACLVLSLTNVSLLPLELLVAGCIPVMNSGDNNTMVLGENDDIAYAEAYPVALAEELCKAVER
SDIDTYANEMSQKYDGVSWENSYRKVEEIIRREVIND
GgcG Protein 2
SEQ ID NO: 13
MTDKIKATVFIPVYNGENDHLEETLTALYTQKTDFSWNVMITDSESKDRSVAIIETFAER
YGNLQLIKLKKSDYSHGATRQMAAELSSAEYMVYLSQDAVPANEHWLAEMLKPFTIHHDIV
AVLGKQKPRIGCFPAMKYDINAVFNEQGVAGAITLWTRQEESLKGKYTKESFYSDVCSAAP
RDFLVNEIGYRSVPYSEDYEYGKDILDAGYMKAYNSDAIVEHSNDVLLSEYKQRIFDETYNV
RRNSGVTTPISVSTVLIQFLKSSVKDAMKIVSDQDYSWKRKLYWLAVNPLFHFEKWRGMRL
ANSVDMTKDNSKHSLENSKSKG
SucG
SEQ ID NO: 14
MKRLLLYVHFNKYNRLSPHVLYQLKKMRPLFSNLIFISNSSLNDSDRQELLSSGLVNEVIQR
QNIGFDFAAWRDGMATVGFESLSEYDNVTIMNDTCFGPLWDMKPYFLTYEDDEEVDFWGL
TNNRQTKEFDEHIQSYFISFKKTVLSNETFLHFWRTVQDFTDVQDVIKNYETQVTTGLLKEG
FRYKCIFNTVTADASGMLHADFSYYNPTAILKHQVPFIKVKTIDANQSIAPYLLQVIKNQTDYP
VDLIVSHMSDIHYPDAPYLLSQKYLEKQEESDLKVSEHSIAVHLHVFYVDLLEEFLHAFTSFK
FPFDLYITTDKSEKESEIKAILDSFRVSAKIVVTGNIGRDVLPMLKLKDELSQYDYIGHFHTKK
SKEADFWAGESWRNELIDMLIKPANTIINQFEDPAIGIIIADIPSFFRFNKIVTPLNEHLIAPEMN
KLWEKMNLSKTIDFEQFDTFVMSYGTFVWFKYDALKPLFDLNLKDGDVPKEPLPQNSILHA
VERLLIYIAWDSHFDFRIAKNNVELTPFLDNKLLNDKSNSLPNTYVDFTYMGGIKGALKYIFIG
PARAIKYIYIRTKEKIFNG
SccG
SEQ ID NO: 15
MKRLLLYVHFNKYNRVSSHVVYQLTQMRSLFSKVIFISNSQVADADVKMLREKHLIDDFIQR
QNSGFDFAAWRDGMVFVGFDELVTYDSVTTMNDTCFGPLWEMYSIYQEFETKTTVDFWG
LTNNRATKSFREHIQSYFISFKASVLRSTAFRDFWENIKEYQDVQKVIDQYETKVTTTLLDAG
FQYDVVFDTTKEDASHMLHADFSYYNPTAILNHRVPFIKVKAIDNNQHITPYLLNDIQKNSTY
PIDLIVSHMSEINYPDFSYLLGHKYVKKRERVDLKNQKVAVHLHVFYVDLLEEFLTAFKQFHF
SYDLFITTDSDDKKAEIEEILSANGQEAQVFVTGNIGRDVLPMLKLKNYLSAYDFVGHFHTKK
SKEADFWAGQSWREELIDMLVKPADNILAQLQQNPKIGLVIADMPTFFRYNKIVDAWNEHLI
APEMNTLWQKMGMTKKIDFNAFHTFVMSYGTFVWFKYDALKPLFDLNLTDDDVPEEPLPQ
NSILHAIERLLIYIAWNEHYDFRISKNPVDLTPFIDNKLLNERGNSAPNTFVDFNYMGGIKGAF
KYIFIGPARAVKYILKRSLQKIKS
GacA
SEQ ID NO: 16
MLENTKILRKVFYLWQKGELMILITGSNGQLGTELRYLLDERGVDYVAVDVAEMDITNEDKV
EAVFAQVKPTLVYHCAAYTAVDAAEDEGKALNEAINVTGSENIAKACGKYGATLVYISTDYV
FDGNKPVGQEWVETDHPDPKTEYGRTKRLGELAVERYAEHFYIIRTAWVFGNYGKNFVFT
MEQLAENHSRLTVVNDQHGRPTWTRTLAEFMCYLTENQKAFGYYHLSNDAKEDTTWYDF
AKEILKDKAVEVVPVDSSAFPAKAKRPLNSTMNLDKAKATGFVIPTWQEALKAFYQQGLKK
GacH
SEQ ID NO: 17
MIKDTFLKTNWLNISHHIILLVFGFYFSFYSLAKELVSSTAQPVNYYAHLLNVSFVGYII
SLIGLSYYLSRQVSRQLFLKTSFIVISYLIVSYWVQITQHLNDKRFDIWSLTKNQFYQFQ
ALPSLLIILVMATLIKILVAYFAIEKDRFGLLGYQGNTFSVALILAVVPINDIHLLKLIS
SRFSELVTAGNSQIALLKISGLLIVLLVIFATIIYVVLNALKHLKSNKPSFSVAATTSLF
LALVFNYTFQYGVKGDEALLGYYVFPGATLFQIVAITLVALLAYVITNRYWPTTFFLLIL
GTIISVVNDLKESMRSEPLLVTDFVWLQELGLVTSFVKKSVIVEMVVGLAICIVVAWYLH
GRVLAGKLFMSPVKRASAVLGLFIVSCSMLIPFSYEKEGKILSGLPIISALNNDNDINWL
GFSTNARYKSLAYVWTRQVTKKIMEKPTNYSQETIASIAQKYQKLAEDINKDRKNNIADQ
TVIYLLSESLSDPDRVSNVTVSHDVLPNIKAIKNSTTAGLMQSDSYGGGTANMEFQTLTSLP
FYNFSSSVSVLYSEVFPKMAKPHTISEFYQGKNRIAMHPASANNFNRKTVYSNLGFSKFLAL
SGSKDKFKNIENVGLLTSDKTVYNNILSLINPSESQFFSVITMQNHIPWSSDYPEEIVAEGKN
FTEEENHNLTSYARLLSFTDKETRAFLEKLTQINKPITVVFYGDHLPGLYPDSAFNKHIENKY
LTDYFIWSNGTNEKKNHPLINSSDFTAALFEHTDSKVSPYYALLTEVLNKASVDKSPDSPEV
KAIQNDLKNIQYDVTIGKGYLLKHKTFFKISR
Group B RMID
SEQ ID NO: 18
MILITGANGQLGSELRHLLDERTQEYVAVDVAEMDITNAEMVDKVFEEVKPSLVYHCAAYTA
VDAAEDEGKELDFAINVTGTENVAKAAAKHDATLVYISTDYVFDGEKPVGQEWEVDDLPDP
KTEYGRTKRMGEELVEKYASKFYTIRTAWVFGNYGKNFVFTMQNLAKTHKTLTVVNDQHG
RPTWTRTLAEFMTYLAENQKDFGYYHLSNDAKEDTTWYDFAVEILKDTDVEVKPVDSSQFP
AKAKRPLNSTMSLEKAKATGFVIPTWQDALKEFYKQEVKK
Group C RMID
SEQ ID NO: 19
MILITGSNGQLGTELRYLLDERHVDYVAVDVAEMDITDADKVEAVFAQVKPTLVYHCAAYTA
VDAAEDEGKALNEAINVTGSENIAKACGKYGATLVYISTDYVFDGNKPVGQEWLETDVPDP
QTEYGRTKRLGELAVEQYAEHFYIIRTAWVFGNYGKNFVFTMQQLAEKHPRLTVVNDQHG
RPTWTRTLAEFMCYLAENQKAFGYYHLSNDAKEDTTWYDFAKEILKDKAVEVVPVDSSAFP
AKAKRPLNSTMNLDKAKATGFVIPTWQEALKEFYQQDRHQ
Group G RMID
SEQ ID NO: 20
MILITGSNGQLGTELRYLLDERHVDYVAVDVAEMDITDADKVEAVFAQVKPTLVYHCAAYTA
VDAAEDEGKALNEAINVTGSENIAKACGKYGATLVYISTDYVFDGNKPVGQEWLETDVPDP
QTEYGRTKRLGELAVEQYAEHFYIIRTAWVFGNYGKNFVFTMQQLAEKHPRLTVVNDQHG
RPTWTRTLAEFMCYLAENQKAFGYYHLSNDAKEDTTWYDFAKEILKDKAIEVVPVDSSAFP
AKAKRPLNSTMNLDKAKATGFVIPTWQEALKEFYQQDRHQ
RmID S. mutans
SEQ ID NO: 21
MILITGSNGQLGTELRHLLNERNEDYVAVDVAEMDITKAEKVDEVFLQVKPSLVYHCAAYTA
VDAAEDEGKELDYAINVTGTENIAKACEKYNATLVYISTDYVFDGEKPVGQEWEVDDKPDP
KTEYGRTKRLGEEAVEKYVKNFYIIRTAWVFGNYGKNFVFTMQHLAKSHNSLTVVNDQHGR
PTWTRTLAEFMTYLAENQKEYGYYHLSNDATEDTTWYDFALEILKDTDVVVKPVDSSQFPA
KAKRPLNSTMSLTKAKATGFVIPTWQEALQEFYKQDVKK
RmID S. uberis
SEQ ID NO: 22
MILITGSNGQLGTELRYLLDERNVEYVAVDVAEMDITNPDMVDEVFAQVKPTLVYHCAAYTA
VDAAEDEGKALNQAINVDGTVNIAKACQKYNATLVYISTDYVFDGTKTVGQEWLETDIPDPK
TEYGRTKRLGEEAVEKYVDQFYIIRTAWVFGHYGKNFVFTMQNLAKTHPKLTVVNDQYGRP
TWTRTLAEFMCHLTENQKDYGYYHLSNDSKEDTSWYDFAKEILKDTDVEVVPVDSSAFPAK
AKRPLNSTMNLDKAKATGFVIPTWQEALNEFYKQEVKK
GccD
SEQ ID NO: 23
MNFLTKKNRILLREMVKTDFKLRYQGSAIGYLWSILKPLMMFTIMYLVFIRFLRLGGNIPHFPV
ALLLANVIWSFFSEATSMGMVSIVSRGDLLRKLNFSKHIIVFSAILGALINFLINLVVVLIFALING
VTISNYAYFSFFLFIELVVFVVGIALLLSTVFVYYRDLAQVWEVLLQAGMYATPIIYPITFVLEG
HPLAAKILMLNPIAQMIQDFRYLLIDRANVTIWQMSTNWFYIAIPYLIPFILLFIGITVFKKNATKF
AEII
GccE
SEQ ID NO: 24
MTNNKIAVKVEHVSKSFKLPTEATKSFRTTLVNRFRGIKGFTEQQVLKDINFEVHKGDFFGIV
GRNGSGKSTLLKIISQIYVPEKGQVTVDGKMVSFIELGVGFNPELTGRENVYMNGAMLGFT
KEEINAMYDDIVDFAELHDFMNQKLKNYSSGMQVRLAFSVAIKAQGDVLILDEVLAVGDEAF
QRKCNDYFMERKDSGKTTILVTHDMGAVKKYCNRAVLIEDGLVKAYGEPFDVANQYSVDN
TETKEELQDSEKVAISDIVQQLRVNLTSKQRITPKEIISFEVSYEVLRDEPTYIAFSLTDMDRNI
WVYNDNSRDQLVEGIGKKTISYQCHLSHLNDIKLKLEVTVRDKDGQMLLFSTAEQSPKIIIQR
DDITSDDFSALDSASGLYQRNGQWTFS
GccF
SEQ ID NO: 25
MHKVSIICTNYNKAPWLGEALDSFLNQKTNFEVDIIVIDDASTDESKTILEDYQTRFPEK
ITLLFNDHNLGITKTWIKACLYAKGKYIARCDGDDYWTDDLKLQKQVDALEASKYSKWSNTD
FDFVDNKGKVLHSNVFETGYIPFTDTYEKVLALKGMTMASTWVVDAELMRFVNQKINIETPD
DTFDMQLELFQLTSLTYINDSTTVYRMTSNSDSRPADKKRMIHRIKQLLQTQVFYLAKYPQA
NIPQIANLLMEQDGKNELRIHELSCLINDLRQELNEKTEQQKEREFEIKEIIENQSRQICELTH
QYNCVINSRRWKYMSKLIDFIRRKK
GgcD
SEQ ID NO: 26
MNFLTKKNRILLREMVKTDFKLRYQGSFIGHLWSILKPMLLFTIMYLVFVRFLKFDDGTPHYA
VSLLLGMVTWNFFTEATNMGMLSIVSRGDLLRKINFPKEIIVISSVVGATINYFINILVVFAFALI
NGVQPSFGVFILIPLFLELFLFATGVAFILATLFVKYRDMGPIWEVMLQAGMYGTPHYSITYIIQ
RGHLGIAKVMMMNPLAQIIQELRHFIVYSGATINWDIFENKFFTLIPIILSLSAFVIGYVIFKRNA
KKFAEIL
GcgE
SEQ ID NO: 27
MSEKKVVLSVDSVSKSFKLPTEASNSLRTSLVNYFKGIKGYTEQHVLDDISFQVEEGDFFGI
VGRNGSGKSTLLKIISKIYEPEKGTVTVDGKLVPFIELGVGFNPELTGRENVFMNGALLGFSR
DEVAAMYDDIVSFAELHDFMDQKLKNYSSGMQVRLAFSIAIKAKGDILILDEVLAVGDEAFQR
KCFDYFAQLKREHKTVILVTHSMEQVQRFCNKAMLIDKGHHMEVGTPLEISQIYKQLNGLNV
AKESAKETENNGISLSSQFINHKDDTLTFTFDVHFEQTIEDPVLTFTIHKDTGELLYRWVSDE
EVEGSIMIKNHKVSIDFAIQNIFPNGKFTTEFGVKSRDRSKEYAMFSGICNFELINRGKSGNNI
YWKPETTVKLS
GgcF
SEQ ID NO: 28
MRMYQGKRFLLTHIWLRGFSGAEINILELATYLKEAGAQVEVFTFLAKSPMLDEFQKNGIPVI
DDSDYPFDVSQYDVVCSAQNIIPPAMIEALGKSQEKLPKFIFFHMAALPEHVLEQPYIYQLEK
KISSATLAISEEIVNKNLKRFFKDIPNLHYYPNPAPESYAAMEHLKKQSPERILVISNHPPQEVI
DMEPLLAKKGIHVDYFGVWSDHYELVTPELLASYDCVVGIGKNAQYCLVMGKPIYIYDHFKG
PGYLTETNFEAAALNNFSGRGFEEQEKTAEELVDDLLEHYQSAQAFQHNHLYDYRSRYTIS
TIVDHIYKSINIIPKAIAPLEQVDVEYIKAITLFIRTRLVRLENDVANLWEAVHRYEQLDRKATAK
REALEQLLTAKTTELNLIKTSRMFKLYQLLWRIKGFFFRKEHLKRAK
SccD
SEQ ID NO: 29
MDFFSRKNRILLKELIKTDFKLRYQGSAIGYLWSILKPLMLFAIMYIVFVRFLPLGGDVP
HWPVALLLGNVIWTFFQETTMMGMVSVVTRGDLLRKLNFSKQTIVFSAVSGAAINFGINVIV
VLIFALLNGVTFTFRWNLFLLIPLFLELLLFSTGIAFILSTLYVRYRDIGPVWEVILQ
GGFYGTPIIYSLTYIATRSVVGAKLLLLSPIAQIIQDMRHILIDPANVTIWQMINHKSIA
VIPYLVPIFVFIIGFLVFNYNAKKFAEII
SccE
SEQ ID NO: 30
MTKNNIAVKVDHVSKYFKLPVESTQSLRTALVNRFKGIKGYKKQHVLRDIDFEVEKGDFFGI
VGRNGSGKSTLLKIISQIYVPEQGKVTVDGKLVSFIELGVGFNPELTGRENVYMNGAMLGFT
TEEVDTMYQDIVDFAELQDFMNQKLKNYSSGMQVRLAFSVAIKAQGDVLILDEVLAVGDEA
FQRKCNDYFLERKNSGKTTILVTHDMAAVKKYCNKAVLIDDGLIKAIGEPFDVANQYSLDNT
DQIVEDKQEEEAAVQEEEQIVVDNLEVKLLSANRMTPRDSIRFEISYNVLADVGTYIALSLTD
VDRNIWIYNDNSLDYLSSGSGKKRVFYECHLKSLNDIKLKLEVTVRDKQGQMLAFSSATNTP
IISINRDDLEGDDKSAMDSASGLIQRNGQWQFS
SccF
SEQ ID NO: 31
MVKVSIICTNYNKGSWIGEAIDSFLKQETSFPYEIIIVDDASTDHSVHIIKTYQKQYPDL
IRAFFNQENQGITKTWSDICKKARGQYIARCDGDDYWIDPFKLQKQIDLLETSPESKWSNTD
FDMVDSKGNIIHKDVLKNNIIPFMDSYEKMLALKGMTMASTWLVETKLMLEINDRINKDAVD
DTFNIQLELFKKTKLAFLRDSTTVYRMDAESDSRSKDSEKLAQRFDRLLETQLEYIEKYPDS
DYKKVLEYLLPKHNDFEKVLAQDGKNVWDNQQITIYLAKGDDQEFSEENCFQFPLQHSGNI
QLTFPENIRKIRIDLSEIPSYYRQVSLVNTTVNTELLPTWTNAKVFGYSYYFI
APDPQMIYDLTAQEGQDFKLTYEWFNVDQPSQPDFLANHLVKELDQKKVELKMLSPYKYQ
YQKAVAERDLYLEQLNEMVVRYNSVTHSRRWTIPTKIINLFRRKK
SucD
SEQ ID NO 32
MELFSKKNRILLKELVKTDFKLRYQGSAIGYLWSILKPLLMFTIMYLVFIRFLRLGGSVPHFPV
ALLLANVIWSFFSEATGMGMVSIVTRGDLLRKLNFSKHTIVFSAVLGALINFSINLVVVLIFALI
NGVTISPFAYMAIPLFIELLILAVGVALLLSTLFVYYRDLAQVWEVLMQAAMYATPIIYPITFVS
DKNPLAAKILMLNPLAQMIQDLRFLLIDRANATIWQMSNHWYYVMIPYLIPFLVLALGILVFNK
NAKKFAEII
SucE
SEQ ID NO 33
MSTRDIAVKVEHVSKSFKLPTEATKSFRTTLVNRFRGIKGYTEQKVLKDINFEVKKGDFFGIV
GRNGSGKSTLLKIISQIYVPEKGTVTVEGKMVSFIELGVGFNPELTGRENVYMNGAMLGFTQ
EEVDAMYEDIVDFAELHDFMNQKLKNYSSGMQVRLAFSVAIKAQGDVLILDEVLAVGDEAF
QRKCNDYFMERKESGKTTILVTHDMAAVKKYCNRAVLIEDGLVKALGDPDDVANQYSFDNA
IASETVEKKEDGKSTEKKESQLISDFSAQLLTKPQISPDEDITISFSYNVLKNMETHVALSFIDI
DTNLGLYNDNSMSLKTNGQGQKTVTMTCQMSYLNHAKLKLAATVRDKDKHPLAFLPVNEIP
VILIDRKVDASNESEWDANTGILRRSSQWT*
SucF
SEQ ID NO 34
MKKILFVSPTGTLDNGAEISITNLMVLLTQEGYDIINVIPKIKHSTHDAYLHKMRENQIK
VYELDYTNWWWESAPGDKIGHLEDRSAYYQKYIYEIRKIIAEEAVDLVITSTANLFQGALAAA
CERIPHYWIIHEFPLDEFAYYKELIPFIEEYSDKIFTVEGKLTEFLRPLLKESQKLF
PFVPFVNIKKNNNLKTGEETRLISISRINENKNQLELLKAYQSMAEPKPELLFVGDWDDSYKE
KCDDFIQSHQLKTVRFLGHQSNPWNLMTDKDILVLNSKMETFGLVFVEALIQGIPVLASNNY
GYSSVVDYFGCGKLYHLGDEKELVALLNEFVTNFSEEKKKSLTQSFMVEEKYTIEKSYCALL
DAISNENSVKSDRPIWLSQFLGAYNPLSTFSPAGKESISIYYRDENGNWSENQKLVFSLFNR
DSFTFSVPKGMTRIRLDMSERPSYYDKITLVDSDTMTQLLPTNVSGFEENNSFYFNHSDPQ
MEFNVSFSKNNVFQLSYQLANLENIFQDSFLPNQLVQKLLSFKEKQSDLEMLKIENHQLQEK
NKLKQEQLEEMVVRYNSVIHSRRWSIPTKMINFLRRKK
SccH
SEQ ID NO: 35
MKQLKKIWDMLGKQKLLIFIFIFALNVTLRNYDLLIGRRANSSLSFKVISKNFDIMIEHWEALPS
HFKIIGGVCLVIYVLSILGLSFYLSKNLKKTFFIELLLGYGLYIVISYFLAVTRELNNESFKIWDLA
KNHFFQPYFLPTLVLIIVCTLALNYLIRVKMKRSHLSRKMTLLLENFSETEFLLTGLIVSFILSDT
LYVKLLQESLRAYYHKPLAYESLLFLYTLLTLILFSVIVEACFNAYRSIKLNRPNLSLAFVSSLL
FATIFNYAFQYGLKNDADLLGKYIVPGATAYQILVLTAAGFFLYLIINRYLLVTFLIVILGSIITVV
NVLKVGMRNEPLLVTDFAWVTNIRLLARSVNANIIFSTLLILAALILLYLFLRKRLLQGKITENH
RLKVGLISSICLLGFSIFIIFRNEKGSKIVNGIPVISQVNNWVDIGYQGFYSNASYKSLMYVWT
KQVTKSIMDKPSDYSKERILKLAKKYNNVANKINKVRTENISNQTVIYILSESFSDPDRVKGV
NLSRDVIPNIKQIKEKTTSGLMHSDGYGGGTANMEFQSLTGLPYYNFNSSVSTLYTEVVPD
MSVFPSISNQFKSKNRVVIHPSSASNYSRKYVYDKLKFPTFVASSGTSDKITHSEKVGLNVS
DKTTYQNILDKINPSQSQFFSVMTMQNHVPWASDEPSDVVATGKGYTKDENGSLSSYARL
LTYTDKETKDFLAQLSQLKHKVTVVFYGDHLPGLYPESAFKKDPDSQYQTDYFIWSNYNTK
TLNHSYVNSSDFTAELLEHTNSKVSPYYALLTEVLDNTTVGHGKLTKEQKEIANDLKLIQYDI
TVGKGYIRNYKGFFDIR
WchF_pHD0486
SEQ ID NO: 36
MKQSVYIIGSKGIPAKYGGFETFVEKLTEYQKDGNIQYYVACMRENSAKSGFTADTFEYNG
AICYNIDVPNIGPARAIAYDIAAVNKAIELSKGNKDEAPIFYILACRIGPFISGLKKKIRSIGGRLL
VNPDGHEWLRAKWSLPVRKYWKFSEQLMVKHADLLVCDSKNIEKYIREDYKQYQPKTTYIA
YGTDTTPSSLKSEDAKVRNWYREKGVSENGYYLVVGRFVPENNYETMIREFIKSKSNKDFV
LITNVEQNKFYDQLLKETGFDKDLRVKFVGTVYDQELLKYIRENAFAYFHGHEVGGTNPSLL
EALASTKLNLLLDVGFNREVGEDGAIYWKKDELAHVIEEVERFDEGDITELDEKSSQRIADAF
TWEKIVSDYEEVFTV
WbbR
SEQ ID NO: 37
MNKYCILVLFNPDISVFIDNVKKILSLDVSLFVYDNSANKHAFLALSSQEQTKINYFSICENIGL
SKAYNETLRHILEFNKNVKNKSINDSVLFLDQDSEVDLNSINILFETISAAESNVMIVAGNPIRR
DGLPYIDYPHTVNNVKFVISSYAVYRLDAFRNIGLFQEDFFIDHIDSDFCSRLIKSNYQILLRK
DAFFYQPIGIKPFNLCGRYLFPIPSQHRTYFQIRNAFLSYRRNGVTFNFLFREIVNRLIMSIFS
GLNEKDLLKRLHLYLKGIKDGLKM
WbbL_pHD0480
SEQ ID NO: 38
MVYIIIVSHGHEDYIKKLLENLNADDEHYKIIVRDNKDSLLLKQICQHYAGLDYISGGVYGFGH
NNNIAVAYVKEKYRPADDDYILFLNPDIIMKHDDLLTYIKYVESKRYAFSTLCLFRDEAKSLHD
YSVRKFPVLSDFIVSFMLGINKTKIPKESIYSDTVVDWCAGSFMLVRFSDFVRVNGFDQGYF
MYCEDIDLCLRLSLAGVRLHYVPAFHAIHYAHHDNRSFFSKAFRWHLKSTFRYLARKRILSN
RNFDRISSVFHP
WbbL
SEQ ID NO: 39
MVAVTYSPGPHLERFLASLSLATERPVSVLLADNGSTDGTPQAAVQRYPNVRLLPTGANLG
YGTAVNRTIAQLGEMAGDAGEPWGDDWVIVANPDVQWGPGSIDALLDAASRWPRAGALG
PLIRDPDGSVYPSARQMPSLIRGGMHAVLGPFWPRNPWTTAYRQERLEPSERPVGWLSG
SCLLVRRSAFGQVGGFDERYFMYMEDVDLGDRLGKAGWLSVYVPSAEVLHHKAHSTGRD
PASHLAAHHKSTYIFLADRHSGWWRAPLRWTLRGSLALRSHL
MVRSSLRRSRRRKLKLVEGRH
RfbF
SEQ ID NO: 40
MNSNIYAVIVTYNPELKNLNALITELKEQNCYVVVVDNRTNFTLKDKLADIEKVHLICLGRNEG
IAKAQNIGIRYSLEKGAEKIIFFDQDSRIRNEFIKKLSCYMDNENAKIAGPVFIDRDKSHYYPIC
NIKKNGLREKIHVTEGQTPFKSSVTISSGTMVSKEVFEIVGMMDEELFIDYVDTEWCLRCLN
YGILVHIIPDIEMVHAIGDKSVKICGINIPIHSPVRRYYRVRNAFLLLRKNHVPLLLSIREVVFSLI
HTTLIIATQKNKIEYMKKHILATLDGIRGITGGGRYNA
WsaD
SEQ ID NO: 41
MDISIIIVNYNTPKLTVEAIESILKSKTKYSYEIIVVDNHSSDDSVRILKGKFPNIVVIENKQNVGF
SKANNQAIKLSKGRYILLLNSDTIVKEDTIEKMIEFMDKSKKVGASGCEVVLPNGELDRACHR
GFPTPEASFYYLVGLARLFPRSRRFNQYHLGYMNLNEPHPIDCLVGAFMMVRREVIEQVGL
LDEEFFMYGEDIDWCYRIKQAGWEIYYCPFTSIIHYKGASSKKKPFKIVYEFHRAMFLFHRKH
YARKYPFIVNCLVYTGIAAKFILSAIINTFRKIGG
WbbP
SEQ ID NO: 42
MKISIIGNTANAMILFRLDLIKTLTKKGISVYAFATDYNDSSKEIIKKAGAIPVDYNLSR
SGINLAGDLWNTYLLSKKLKKIKPDAILSFFSKPSIFGSLAGIFSGVKNNTAMLEGLGFL
FTEQPHGTPLKTKLLKNIQVLLYKIIFPHINSLILLNKDDYHDLIDKYKIKLKSCHILGG
IGLDMNNYCKSTPPTNEISFIFIARLLAEKGVNEFVLAAKKIKKTHPNVEFIILGAIDKE
NPGGLSESDVDTLIKSGVISYPGFVSNVADWIEKSSVFVLPSYYREGVPRSTQEAMAMGRP
ILTTNLPGCKETIIDGVNGYVVKKWSHEDLAEKMLKLINNPEKIISMGEESYKLARERFDANV
NNVKLLKILGIPD
WsaP
SEQ ID NO: 43
MVKVIRGRERFLTKLYAFVDFAMMQGAFFLAWVLKFKVFHNGVGGHLPLEDYLFWSFVYG
AIAIVIGYLVELYAPKRKEKFSNELAKVLQVHTLSMFVLLSVLFTFKTVDVSRSFLLLYFAWNLI
LVSIYRYIVKQSLRTLRKKGYNKQFVLIIGAGSIGRKYFENLQMHPEFGLEVVGFLDDFRTKH
APEFAHYKPIIGQTADLEHVLSHQLIDEVIVALPLQAYPKYREIIAVCEKMGVRVSIIPDFYDILP
AAPHFEIFGDLPIINVRDVPLDELRNRVLKRSFDIVFSLVAIIVTS
PIMLLIAIGIKLTSPGPIIFKQERVGLNRRTFYMYKFRSMKPMPQSVSDTQWTVESDPRRTKF
GAFLRKTSLDELPQFFNVLKGDMSIVGPRPERPFFVEKFKKEIPKYMIKHHVRPGITGWAQV
CGLRGDTSIQERIEHDLFYIENWSLWLDIKIILLTITNGLVNKNAY
WsaC
SEQ ID NO: 44
MEMPLVSIVVATYFPRTDFFEKQLQSLNNQTYENIEIIICDDSANDAEYEKVKKMVENII
SRFPCKVIRNEKNVGSNKTFERLTQEANGDYICYCDQDDIWLSEKVERLVNHITKHHCTLVY
SDLSLIDENDRIIHKSFKRSNFRLKHVHGDNTFAHLINRNSVTGCAMMIRADVAKSAIPFPDY
DEFVHDHWLAIHAAVKGSLGYIKEPLVWYRIHLGNQIGNQRLVNITNINDYIRHRIEKQGNKY
RLTLERLSLTLQQKQLVYFQIHLTEARKKFSQKPCLGNFFKIVPLIKYDIILFLFELMIFTVPFTC
SIWIFKKLKY
WsaE
SEQ ID NO: 45
MERCRMNKKIPFDQYQRYKNAAEIINLIREENQSFTILEVGANEHRNLEHFLPKDQVTYLDIE
VPEHLKHMTNYIEADATNMPLDDNAFDFVIALDVFEHIPPDKRNQFLFEINRVAKEGFLIAAP
FNTEGVEETEIRVNEYYKALYGEGFRWLEEHRQYTLPNLEETEDILRKENIEYVKFEHGSLL
FWEKLMRLHFLVADRNVLHDYRFMIDDFYNKNIYEVDYIGPCYRNFIVVCRDKAKREFIQSIY
EKRKQNSYLKNSTISKLNELENSIYSLKIIDKENQIYKKSLEITEQLLEDLKLKEQQIIEKIQTIKK
KTEMIELQNQKIQELKIECENKSIENNNLYSQLLEKENYIKQ
LQNQAESMRIKNRLKKILNFSFIKYVRKIINIIFRRKFKFKLQPVHHLEWSNGKWLVLGR
DPHFILKGGSYPSSVVTIIQWRASANSSALLRLYYDTGGGFSENQSFNLGKIGNDINRDYECV
ICLPENIHLLRLDIEGEISEFELENLTFTSISRLEVFYKSFINHCRKRNIKNYKELYS
LIKKLFILVRREGLKSIWYRAKQKLSMELLSEDPYEVFLNVSSKVDKEIVLSEIKKLKYK
PKFSVILPVYNVEEKWLRKCIDSVLNQWYPYWELCIVDDNSSKDYIKPVLEEYSNRDSRIKT
VFRSNNGHISEASNTALEIATGDFIALLDHDDELAPEALYENAVLLNEHPDADMIYSDEDKITK
DGKRHSPLFKPDWSPDTLRSQMYIGHLTVYRTNLVRQLGGFRKGFEGSQDYDLALRVAEK
TNNIYHIPKILYSWREIETSTAVNPSSKPYAHEAGLKALNEHLERVFGKGKAWAEETEYLFVY
DVRYAIPEDYPLVSIIIPTKDNIELLSSCIQSILDKTTYPNYEILIMNNNSVMEETYSWFDKQKE
NSKIRIIDAMYEFNWSKLNNHGIREANGEVFVFLNNDTIVISEDWLQRLVEKALREDVGTVG
GLLLYEDNTIQHAGVVIGMGGWADHVYKGMHPVHNTSPFISPVINRNVSASTGACLAIAKKV
IEKIGGFNEEFIICGSDVEISLRALKMGYVNIYDPYVRLYHLESKTRDSFIPERDFELSAKYYS
PYREIGDPYYNQNLSYNHLIPTIRS
WbbQ
SEQ ID NO: 46
MARSGGVVIKKKVAAIIITYNPDLTILRESYTSLYKQVDKIILIDNNSTNYQELKKLFEK
KEKIKIVPLSDNIGLAAAQNLGLNLAIKNNYTYAILFDQDSVLQDNGINSFFFEFEKLVS
EEKLNIVAIGPSFFDEKTGRRFRPTKFIGPFLYPFRKITTKNPLTEVDFLIASGCFIKLE
CIKSAGMMTESLFIDYIDVEWSYRMRSYGYKLYIHNDIHMSHLVGESRVNLGLKTISLHGPLR
RYYLFRNYISILKVRYIPLGYKIREGFFNIGRFLVSMIITKNRKTLILYTIKAIKDG
INNEMGKYKG

1. A method of synthesizing a rhamnose polysaccharide, the method comprising
(i) transferring a rhamnose moiety to a hexose monosaccharide, disaccharide or trisaccharide using a hexose-β-1,4-rhamnosyltransferase, a hexose-α-1,2-rhamnosyltransferase and/or a hexose-α-1,3-rhamnosyltransferase, or an enzymatically active fragment or variant thereof to form a disaccharide, trisaccharide or tetrasaccharide comprising a rhamnose moiety at a non-reducing end of the disaccharide, trisaccharide or tetrasaccharide;
(ii) generating the rhamnose polysaccharide by extending from the rhamnose moiety at the non-reducing end of the disaccharide, trisaccharide or tetrasaccharide using a heterologous bacterial enzyme Streptococcus pyogenes Group A carbohydrate enzyme C (GacC) and/or Streptococcus pyogenes Group A carbohydrate enzyme G (GacG) or an enzymatically active homologue, variant or fragment thereof.

2. The method according to claim 1, wherein the method is performed in a bacterium species heterologous to the bacterium species from which the enzyme GacC and/or GacG, or an enzymatically active homologue, variant or fragment thereof is derived.

3. The method according to claim 1, wherein the hexose-β-1,4-rhamnosyltransferase is not a GlcNAc-β-1,4-rhamnosyltransferase.

4. The method according to claim 1, wherein the hexose-β-1,4-rhamnosyltransferase is a Glc-β-1,4-rhamnosyltransferase or an enzymatically active fragment or variant thereof.

5. The method according to claim 4, wherein the Glc-β-1,4-rhamnosyltransferase comprises a WchF enzyme, or an enzymatically active fragment or variant thereof.

6. (canceled)

7. The method according to claim 1, wherein the hexose-α-1,2-rhamnosyltransferase is a galactose-α-1,2-rhamnosyltransferase or an enzymatically active fragment or variant thereof.

8. The method according to claim 7, wherein the galactose-α-1,2-rhamnosyltransferase comprises a WbbR enzyme, or an enzymatically active fragment or variant thereof.

9. (canceled)

10. The method according to claim 1, wherein the hexose-α-1,3-rhamnosyltransferase is a GlcNAc-α-1,3-rhamnosyltransferase, a diNAcBac-α-1,3-rhamnosyltransferase, a Glc-α-1,3-rhamnosyltransferase, a galactose-α-1,3-rhamnosyltransferase or an enzymatically active fragment or variant thereof.

11. The method according to claim 10, wherein the GlcNAc-α-1,3-rhamnosyltransferase comprises a WbbL enzyme, or an enzymatically active fragment or variant thereof and the galactose-α-1,3-rhamnosyltransferase comprises a WsaD enzyme, or an enzymatically active fragment or variant thereof.

12. (canceled)

13. (canceled)

14. The method according to claim 1, wherein the enzymatically active homologue of GacC and/or GacG is selected from a homologue from a Streptococci Group B, Group C, Group G, S. mutans, S. uberis or an enzymatically active fragment or variant thereof.

15. The method according to claim 1, wherein the method is performed in a gram-negative bacterium/bacteria, such as E. coli.

16. (canceled)

17. The method according to claim 1, wherein step ii) further comprises using one or more additional enzymes from the Gac cluster of bacterial enzymes, or one or more enzymatically active homologue(s), variant(s), or fragment(s) thereof.

18. The method according to claim 1, the method further comprising:
(iii) conjugating the rhamnose polysaccharide to an acceptor molecule using an O-oligosaccharyltransferase capable of recognizing the hexose monosaccharide at the reducing end of the rhamnose polysaccharide to form a rhamnose glycoconjugate.

19. (canceled)

20. The method according to claim 18, wherein the O-oligosaccharyltransferase comprises PglB, PglL, PglS or WsaB, or an enzymatically active homologue, fragment, or variant thereof.

21. The method according to claim 18, wherein the acceptor molecule comprises a peptide or a protein.

22. The method according to claim 18, wherein the method further comprises purifying the rhamnose glycoconjugate.

23. (canceled)

24. A synthetic streptococcal polysaccharide, the polysaccharide having a non-reducing end comprising a linear chain of rhamnose moieties and a reducing end comprising a hexose monosaccharide, disaccharide, or trisaccharide, wherein the polysaccharide comprises a α-1,3 bond or a α-1,2 bond between the hexose monosaccharide, disaccharide, or trisaccharide and the linear chain of rhamnose moieties; or the polysaccharide comprises a β-1,4 bond between the hexose monosaccharide, disaccharide, or trisaccharide and the linear chain of rhamnose moieties and the hexose monosaccharide, disaccharide, or trisaccharide does not comprise N-acetylglucosamine.

25. The synthetic streptococcal rhamnose polysaccharide according to claim 24, wherein the polysaccharide comprises a α-1,3 bond between the hexose monosaccharide, disaccharide, or trisaccharide and the linear chain of rhamnose moieties and the hexose comprises N-acetylglucosamine, N,N′-diacetylbacillosamine, glucose or galactose.

26. The synthetic streptococcal rhamnose polysaccharide according to claim 24, wherein the polysaccharide comprises a α-1,2 bond or a β-1,4 bond between the hexose monosaccharide, disaccharide, or trisaccharide and the linear chain of rhamnose moieties and the hexose comprises galactose.

27. (canceled)

28. The synthetic streptococcal rhamnose polysaccharide according to claim 24, wherein the polysaccharide comprises a polysaccharide or a fragment or variant thereof selected from the group consisting of a Group A, Group B, Group C and Group G carbohydrate.

29. The synthetic streptococcal rhamnose polysaccharide according to claim 24 conjugated to an acceptor.

30. (canceled)

31. (canceled)

32. An immunogenic composition or vaccine comprising the synthetic streptococcal rhamnose polysaccharide according to claim 24, together with a pharmaceutically acceptable and/or sterile excipient, carrier, and/or diluent.

33. (canceled)

34. The immunogenic composition or vaccine according to claim 32, wherein the immunogenic composition or vaccine further comprises an antigen, polypeptide and/or adjuvant.

35. (canceled)

36. A bacterial host cell, the bacterial host cell comprising a hexose-β-1,4-rhamnosyltransferase, a hexose-α-1,2-rhamnosyltransferase or a hexose-α-1,3-rhamnosyltransferase, or an enzymatically active fragment or variant thereof and a heterologous bacterial enzyme GacC and/or GacG or an enzymatically active homologue, variant or fragment thereof.

37. A kit of parts, the kit comprising:
(i) a nucleic acid sequence encoding a hexose-β-1,4-rhamnosyltransferase, a hexose-α-1,2-rhamnosyltransferase or a hexose-α-1,3-rhamnosyltransferase, or an enzymatically active fragment or variant thereof; and
(ii) a nucleic acid sequence encoding a heterologous bacterial enzyme GacC and/or GacG or an enzymatically active homologue, variant, or fragment thereof.
